SEQUENCE LISTING (20 sequences)

SEQ ID NO: 1 - DNA, 29 bp, Artificial Sequence
  Oligonucleotide primer directed to human Factor IX
catggggtcccccnnnatctccttgacat

SEQ ID NO: 2 - DNA, 30 bp, Artificial Sequence
  Oligonucleotide primer directed to human Factor IX
acatggggtcccccnnnatctccttgacat

SEQ ID NO: 3 - DNA, 31 bp, Artificial Sequence
  Oligonucleotide primer directed to human Factor IX
aacatggggtcccccnnnatctccttgacat

SEQ ID NO: 4 - DNA, 30 bp, Artificial Sequence
  Oligonucleotide primer directed to human Factor IX
catggggtcccccnnnatctccttgacatg

SEQ ID NO: 5 - DNA, 31 bp, Artificial Sequence
  Oligonucleotide primer directed to human Factor IX
acatggggtcccccnnnatctccttgacatg

SEQ ID NO: 6 - DNA, 32 bp, Artificial Sequence
  Oligonucleotide primer directed to human Factor IX
aacatggggtcccccnnnatctccttgacatg

SEQ ID NO: 7 - DNA, 31 bp, Artificial Sequence
  Oligonucleotide primer directed to human Factor IX
catggggtcccccnnnatctccttgacatga

SEQ ID NO: 8 - DNA, 32 bp, Artificial Sequence
  Oligonucleotide primer directed to human Factor IX
acatggggtcccccnnnatctccttgacatga

SEQ ID NO: 9 - DNA, 33 bp, Artificial Sequence
  Oligonucleotide primer directed to human Factor IX
aacatggggtcccccnnnatctccttgacatga

SEQ ID NO: 10 - DNA, 29 bp, Artificial Sequence
  Oligonucleotide primer directed to human Factor IX
ccagaagcgcaatnnnatgattgtactta

SEQ ID NO: 11 - DNA, 30 bp, Artificial Sequence
  Oligonucleotide primer directed to human Factor IX
tccagaagcgcaatnnnatgattgtactta

SEQ ID NO: 12 - DNA, 31 bp, Artificial Sequence
  Oligonucleotide primer directed to human Factor IX
ttccagaagcgcaatnnnatgattgtactta

SEQ ID NO: 13 - DNA, 30 bp, Artificial Sequence
  Oligonucleotide primer directed to human Factor IX
ccagaagcgcaatnnnatgattgtacttat

SEQ ID NO: 14 - DNA, 31 bp, Artificial Sequence
  Oligonucleotide primer directed to human Factor IX
tccagaagcgcaatnnnatgattgtacttat

SEQ ID NO: 15 - DNA, 32 bp, Artificial Sequence
  Oligonucleotide primer directed to human Factor IX
ttccagaagcgcaatnnnatgattgtacttat

SEQ ID NO: 16 - DNA, 31 bp, Artificial Sequence
  Oligonucleotide primer directed to human Factor IX
ccagaagcgcaatnnnatgattgtacttatt

SEQ ID NO: 17 - DNA, 32 bp, Artificial Sequence
  Oligonucleotide primer directed to human Factor IX
tccagaagcgcaatnnnatgattgtacttatt

SEQ ID NO: 18 - DNA, 33 bp, Artificial Sequence
  Oligonucleotide primer directed to human Factor IX
ttccagaagcgcaatnnnatgattgtacttatt

SEQ ID NO: 19 - DNA, 33 bp, Artificial Sequence
  Oligonucleotide primer directed to human Factor IX
accagtttcaacacannnggcagcagttacatt

SEQ ID NO: 20 - DNA, 29 bp, Artificial Sequence
  Oligonucleotide primer directed to human Factor IX
atggggtcccccagcatctccttgacatg

(19) United States
(12) Patent Application Publication
(10) Pub. No.: US 2005/0250688 A1
(43) Pub. Date: Nov. 10, 2005

(54) Methods for treating an ischemic disorder and improving stroke outcome

(75) Inventors: David J. Pinsky, Cresskill, NJ (US); David Stern, Great Neck, NY (US); Ann Marie Schmidt, Franklin Lakes, NJ (US); Eric Rose, Tenafly, NJ (US); Robert A. Solomon, Palisades, NY (US)

Correspondence Address: John P. White, Esq.;cooper & Dunham LLP, 1185 Avenue of the Americas, New York, NY, 10036, US

(73) Assignee: The Trustees of Columbia University in the City of New York

(21) Appl. No.: 10/692,439

(22) Filed: Oct. 22, 2003

Related U.S. Application Data
(63) Continuation of application No. 09/671,100, filed on Sep. 27, 2000, now abandoned.
Application No. 09/671,100 is a continuation of application No. PCT/US99/07175, filed on Apr. 1, 1999.

(51) Int. Cl. A61K 48/00; A61K 39/395; A61K 38/37

(52) U.S. Cl. 514/12; 424/145.1; 514/44

(57) ABSTRACT

The present invention provides for a method for treating an ischemic disorder in a subject which comprises administering to the subject a pharmaceutically acceptable Factor IXa compound in a sufficient amount over a sufficient time period so as to treat the ischemic disorder in the subject. The invention further provides a method for treating an ischemic disorder in a subject which comprises administering to the subject a pharmaceutically acceptable form of inactivated Factor IXa in a sufficient amount over a sufficient period of time to inhibit coagulation so as to treat the ischemic disorder in the subject.

This application is a continuation-in-part of U.S. Ser. No. 09/053,871, filed Apr. 1, 1998 which is a is a continuation-in-part of PCT International Application No. PCT/US97/17229, filed. Sep. 25, 1997, which is a continuation-in-part of U.S. Ser. No. 08/721,447, filed Sep. 27, 1996 which applications are hereby incorporated by reference in their entireties.

The invention disclosed herein was made with Government support under National Institutes of Health, National Heart, Lung and Blood Institute award HL55397 of the Department of Health and Human Services. This study was also supported in part by the US Public Health Service (R01 HL59488, R01 HL55397, and K08 NS02038). Accordingly, the U.S. Government has certain rights in this invention.

Throughout this application, various publications are referenced following certain Examples and within the Detailed Description of the Invention section. The disclosures of these publications in their entireties are hereby incorporated by reference into this application in order to more fully describe the state of the art as known to those skilled therein as of the date of the invention described and claimed herein.

### BACKGROUND OF THE INVENTION

As described in Colman et al., Editors, Hemostasis and Thrombosis, Third Edition, J.B. Lippincott Company, Philadelphia, 1994, pages 33-36, 62-63 and 94-105, human Factor IX is a 415 amino acid glycoprotein (Mr=57,000, 17% carbohydrate). Factor IX is a proenzyme that has no catalytic activity. During the coagulation cascade, it is cleaved by Factor XIa to produce catalytically active Factor IXa. A wide variety of Factor IX gene mutations are found in patients with hemophilia B. Among these are mutations in the enzyme active site, including a Ser365 to Arg mutation and mutations near His221. (Colman et al., page 63) These mutations affect the ability of the active site to proteolytically cleave its Factor X substrate. Mutations of Gly363 to Val were found to be functionally normal but unable to activate Factor X (Colman et al., page 104).

The gene for Factor IX has been identified, cDNA for Factor IX has been isolated, sequenced, and cloned into expression vectors, and recombinant Factor IX has been expressed. See, for example, Durachi et al., “Isolation and characterization of a cDNA coding for human Factor IX,” Proc. Natl. Acad. Sci. USA 79: 6461, 1982 (GenBank Accession Nos. J00136 and 18290); Choo et al., “Molecular cloning of the gene for human anti-haemophilic factor Factor IX,” Nature 299: 178, 1982; Anson et al., “the gene structure of human anti-haemophilic factor Factor IX,” EMBO J. 3:1053, 1984; Yshitake et al., “Nucleotide Sequence of the gene for human Factor IX,” Biochemistry 24:3736, 1985 (GenBank Accession No. 182,613); Anson et al., “Expression of active human clotting Factor IX from recombinant DNA clones in mammalian cells,” Nature 315:683,1985; Busby et al., “Expression of active human Factor IX in transfected cells,” Nature 316:271, 1985; de la Salle et al., “Active gamma carboxylated human Factor IX expressed using recombinant DNA techniques,” Nature 316: 268, 1985; and Kaufman et al., “Expression, purification, and characterization of recombinant gamma-carboxylated factor IX synthesized in Chinese hamster ovary cells,” J. Biol. Chem. 261:9622, 1986. See also Brownlee et al., UK Patent Application GB 2 125 409 A, published Mar. 7, 1984; Anson et al., U.S. Pat. No. 5,171,569, issued Dec. 15, 1992; Muelien U.S. Pat. No. 5,521,070, issued May 28, 1996; Kaufman et al., U.S. Pat. No. 4,770,999, issued Sep. 13, 1988; and Barr et al., U.S. Pat. No. 5,460,950, issued Oct. 24, 1995.

In addition, Benedict et al. (1994) Texas Heart Institute Journal Vol 21, No. 1, pp 85-90 disclose that infusion of Factor IXai at concentrations sufficient to inhibit intravenous coagulation did not produce bleeding significantly different from that in control animals. Therefore, the invention disclosed herein was unexpected in view of this report.

### SUMMARY OF THE INVENTION

The present invention provides a method for treating an ischemic disorder in a subject which comprises administering to the subject a pharmaceutically acceptable form of a Factor IXa compound in a sufficient amount over a sufficient period of time to inhibit coagulation so as to treat the ischemic disorder in the subject. The present invention provides a method for treating an ischemic disorder in a subject which comprises administering to the subject a pharmaceutically acceptable form of inactivated Factor IXa in a sufficient amount over a sufficient period of time to inhibit coagulation so as to treat the ischemic disorder in the subject.

### BRIEF DESCRIPTION OF THE FIGURES

FIGS. 1A, 1B, 1C and 1D. Overview of operative setup for murine focal cerebral ischemia model. FIG. 1A. Suture based retraction system is shown in the diagram. FIG. 1B. View through the operating microscope. The large vascular stump represents the external carotid artery, which is situated inferomedially in the operating field. FIG. 1c. Photograph of heat-blunted occluding suture of the indicated gauge (5-0 [bottom] or 6-0 nylon [top]). FIG. 1D. Schematic diagram of murine cerebrovascular anatomy, with thread in the anterior cerebral artery, occluding the middle cerebral artery at its point of origin.

FIG. 2. Comparison of cerebrovascular anatomy between strains of mice. Following anesthesia, mice were given an intracardiac injection of India ink followed by humane euthanasia. An intact Circle of Willis can be observed in all strains, including bilateral posterior communicating arteries, indicating that there are no gross strain-related differences in cerebrovascular anatomy.

FIGS. 3A, 3B and 3C. Effects of mouse strain on stroke outcome. Mice (20-23 gm males) were subjected to 45 minutes of MCA occlusion (using 12 mm 6.0 occluding suture) followed by 24 hours of reperfusion, and indices of stroke outcome determined. FIG. 3A. Effects of strain on infarct volume, determined as a percentage of ipsilateral hemispheric volume, as described in the Methods section. FIG. 3B. Effects of strain on neurological deficit score, graded from no neurologic deficit (O) to severe neurologic deficit (4), with scores determined as described in the Methods section. FIG. 3C. Effects of strain on cerebral blood flow, measured by laser doppler flowmetry as relative flow over the infarcted territory compared with blood flow over the contralateral (noninfarcted) cortex. Strains included 129J (n=9), CD1 (n=11), and C57/B16 mice (n=11); *=p<0.05 vs 129J mice.

FIGS. 4A, 4B and 4C. Effects of animal size and diameter of the occluding suture on stroke outcome. Male CD-1 mice of the indicated sizes were subjected to middle cerebral artery occlusion (45 minutes) followed by reperfusion (24 hours) as described in the Methods section. Suture size (gauge) is indicated in each panel. Small animals (n=11) were those between 20-25 gm (mean 23 gm), and large animals were between 28-35 gm (mean 32 gm; n=14 for 6.0 suture, n=9 for 5.0 suture). FIG. 4A. Effects of animal/suture size on infarct volume, FIG. 4B. neurological deficit score, and FIG. 4C. cerebral blood flow, measured as described in FIG. 3. P values are as shown.

FIGS. 5A, 5B and 5C. Effects of temperature on stroke outcome. Male C57/B16 mice were subjected to 45 minutes of MCA occlusion (6.0 suture) followed by reperfusion. Core temperatures were maintained for 90 minutes at 37° C. (normothermia, n=11) using an intrarectal probe with a thermocouple-controlled heating device. In the second group (hypothermia, n=12), animals were placed in cages left at room temperature after an initial 10 minutes of normothermia (mean core temperature 31° C. at 90 minutes). In both groups, after this 90 minute observation period, animals were returned to their cages with ambient temperature maintained at 37° C. for the duration of observation. Twenty-four hours following MCA occlusion, indices of stroke outcome were recorded; FIG. 5A. infarct volume, FIG. 5B. neurological deficit score, and FIG. 5C. cerebral blood flow, measured as described in FIG. 3. *=p<0.05 values are as shown.

FIGS. 6A, 6B and 6C. Outcome comparisons between permanent focal cerebral ischemia and transient focal cerebral ischemia followed by reperfusion. The MCA was either occluded permanently (n=11) or transiently (45 minutes, n=17) with 6.0 gauge suture in 22 gram Male C57/B16 mice, as described in the Methods section. Twenty-four hours following MCA occlusion, indices of stroke outcome were recorded; FIG. 6A. infarct volume, FIG. 6B. neurological deficit score, and FIG. 6C. cerebral blood flow, measured as described in FIG. 3.

FIGS. 7A-7F. FIG. 7A. Effect of stroke and Factor IXai administration in stroke on the accumulation of radiolabeled platelets. ¹¹¹Indium-platelets were administered either in control animals without stroke (n=4), or in animals immediately prior to stroke with (n=7) or without preoperative administration of Factor IXai (300 μg/kg, n=7). Platelet accumulation is expressed as the ipsilateral cpm/contralateral cpm. Means±SEM are shown. *p<0.05 vs No Stroke; **p<0.05 vs Stroke+Vehicle. FIG. 7B. Accumulation of fibrin in infarcted cerebral tissue. Twenty-two hours following focal cerebral ischemia and reperfusion, a brain was harvested from a representative mouse which had been pretreated prior to surgery with either vehicle (leftmost two lanes) or Factor IXai (300 μg/kg, rightmost two lanes). The brains were divided into ipsilateral (R) and contralateral (L) hemispheres, and plasmin digestion performed to solubilize accumulated fibrin. Immunoblotting was performed using a primary antibody directed against a neoepitope expressed on the gamma-gamma chain dimer of crosslinked fibrin. FIG. 7C-7F. Immunohistochemical identification of sites of fibrin formation in stroke. Using the same antibody as described in FIG. 2B to detect fibrin, brains were harvested from two mice following stroke (upper and lower panels each represent a mouse). Arrows identify cerebral microvessels. Note that in both ipsilateral hemispheres (left, FIGS. 7C and 7E), intravascular fibrin can be clearly identified by the red stain, which is not seen in the contralateral (right, FIGS. 7D and 7F), nonischemic hemispheres.

FIGS. 8A-8C. FIG. 8A. Effect of Factor IXai on relative CBF in a murine stroke model, measured by laser doppler. CBF in Factor IXai-treated animals (300 μg/kg, n=48, dashed line) is significantly higher at 24 hours than vehicle-treated controls (n=62). Means±SEM are shown. *pc0.05.

FIG. 8B. Effect of Factor IXai on infarct volumes in a murine stroke model, measured by TTC—staining of serial coronal sections. Animals were given vehicle (n=62) or Factor IXai (300 μg/kg, n=48). Means±SEM are shown. *pk0.05. FIG. 8C. Dose-response of Factor IXai in stroke. Factor IXai was administered immediately prior to the onset of stroke, and cerebral infarct volumes determined as described in FIG. 8B above. N=62, 48, 6, and 6, for Vehicle, 300 μg/kg, 600 μg/kg, and 1200 μg/kg doses respectively. Means±SEM are shown. *p<0.05 vs vehicle-treated animals.

FIGS. 9A-9B. Effect of Factor IXai on Intracerebral hemorrhage. FIG. 9A. Spectrophotometric hemoglobin assay was performed as described in the Methods section. O.D. at 550 nm is linearly related to brain hemoglobin content^{11,12} (see references following example in which figure is discussed). FIG. 9B. Visually-determined ICH score by a blinded observer, as described in the methods section. ICH score correlates with spectrophotometrically-determined brain hemoglobin content ^{11,12}. Means±SEM are shown. *p<0.05 vs vehicle-treated animals.

FIG. 10. Effect of timing of Factor IXai administration on cerebral infarct volumes when given after the onset of stroke. Mice were subjected to focal cerebral ischemia and reperfusion as described in the Methods section. The preocclusion administration (leftmost 2 bars) data is that shown FIG. 8B. In additional experiments to determine the effects of Factor IXai administered after stroke, immediately following withdrawal of the intraluminal occluding suture, vehicle (normal saline, n=13) or Factor IXai (300 μg/kg, n=7) was administered intravenously. Cerebral infarct volumes (based on TTC-stained serial sections obtained at 22 hrs) were determined. Means±SEM are shown. *p<0.05, **p<0.05 vs vehicle-treated animals.

FIGS. 11A-11D FIG. 1A. Effect of stroke on the accumulation of radiolabeled platelets, and the inhibitory effects of Factor IXai. ¹¹¹Indium-platelets were administered to either control animals without stroke (n=4), or to animals immediately prior to stroke treated with vehicle (n=7) or with preoperative administration of Factor IXai (300 μg/kg, n=7). Platelet accumulation at 24 hours is expressed as the ipsilateral cpm/contralateral cpm. Means±SEM are shown. *p<0.05 vs No Stroke and vs Stroke+IXai. FIG. 11B. Accumulation of fibrin in infarcted cerebral tissue. After 45 minutes of right middle cerebral artery occlusion and 23 hours of reperfusion, brains were harvested from representative mice which had been treated prior to surgery with either vehicle (leftmost two lanes) or Factor IXai (300 μg/kg, rightmost two lanes). The brains were divided into ipsilateral (R) and contralateral (L) hemispheres, and plasmin digestion performed to solubilize accumulated fibrin. Immunoblotting was performed using a primary antibody directed against a neoepitope expressed on the gamma-gamma chain dimer of crosslinked fibrin. FIG. 11c. Immunohistochemical identification of sites of fibrin formation in stroke. Using the same procedures as described in FIG. 11b, brains were harvested at 24 hours, formalin fixed/paraffin embedded, and fibrin was detected immunohistochemically using the primary antibody used for immunoblotting (FIG. 11B). Arrows identify cerebral microvessels, with fibrin (red staining) observed in the in the ipsilateral microvasculature (right panel), but not in the contralateral (nonischemic, left panel) microvasculature. Cerebral microvessels, shown in the center of each field, stained prominently for fibrin (sepia color) in the ipsilateral hemisphere of vehicle-treated animals (top right panel). In contrast, microvessels from the ipsilateral hemisphere of Factor IXai-treated mice rarely demonstrated intravascular fibrin (bottom right panel). FIG. 11D. Effect of Factor IXai on CBF in a murine stroke model. Serial measurements of relative CBF were made using a laser doppler over precisely defined neuroanatomic landmarks (13), expressed as ipslateral/contralateral CBF; Experiments were performed as described in FIG. 1b; n=48 for Factor IXai-treated animals (300 μg/kg); n=62 for vehicle-treated animals subjected to identical procedures. Means±SEM are shown. *p<0.05.

FIG. 12. Modified cephalin clotting time to examine the antithrombotic effects of intravenous Factor IXai and heparin. Factor IXai (300 μg/kg, n=5) or heparin (50 U/kg, n=4, or 100 U/kg, n=3) was administered to mice as an intravenous single bolus, plasma obtained, and the time to clot formation measured in an in vitro reaction in which the activity of Factor IXa is rate-limiting.

FIGS. 13A-13C. Effect of Factor IXai on infarct volume an intracerebral hemorrhage in a murine stroke model. FIG. 13A. Effect of Factor IXai on cerebral infarct volumes, measured by TTC staining of serial coronal sections of brain. Prior to stroke, animals were given either vehicle (n=62), Factor IXai at 150 μg/kg (n=5), 300 μg/kg (n=48), 600 μg/kg (n=6), or 1200 μg/kg (n=6), or heparin at 50 U/kg (n=14) or 100 U/kg (n=15). Means±SEM are shown. *p<0.05 vs vehicle-treated animals. FIG. 13B. Effect of Factor IXai on intracerebral hemorrhage 24 hours after stroke, as measured by a quantitative spectrophotometric hemoglobin assay (17, see references following Example 4), in which O.D. at 550 nm is linearly related to brain hemoglobin content. Relative O.D. was determined as the ratio of the O.D. of a given experiemtnal condition relative to the mean O.D. of vehicle-treated animals. Prior to stroke, animals were given either vehicle (n=9), Factor IXai at 150 μg/kg (n=4), 300 μg/kg (n=9), 600 μg/kg (n=3), or 1200 μg/kg (n=3), or heparin at 50 U/kg (n=5) or 100 U/kg (n=11). Means±SEM are shown. *p<0.05 vs vehicle-treated animals. FIG. 13C. Infarct volume/ICH plot of data shown in FIGS. 13A and 13B. Infarct volumes were plotted against intracerebral hemorrhage to display the how a given agent at a given dose effects both infarct volume and ICH simultaneously. V=vehicle, H=heparin, and IXai=Factor IXai; doses are shown. Significant values are shown in FIGS. 13A and 13B, but are omitted here for clarity.

FIG. 14. Effect of timing of Factor IXai administration on cerebral infarct volumes. Mice were either pretreated with intravenous vehicle (n=62) or Factor IXai (300 μg/kg, n=48) prior to focal cerebral ischemia and reperfusion, or immediately upon withdrawal of the intraluminal middle cerebral arterial occluding suture (n=13 for vehicle, n=7 for Factor IXai). Cerebral infarct volumes were determined from TTC-stained serial cerebral sections. Means±SEM are shown. *p<0.05 vs similarly vehicle-treated animals. (The preocclusion administration data is the same data that is shown in FIG. 13A for the 300 μg/kg dose, but is repeated here to facilitate comparison with the postreperfusion data.

FIGS. 15A-15B. Modified cephalin clotting time to evaluate the antithrombotic effects of intravenous Factor IXai. 15a. Antithrombotic effects of Factor IXai as compared to heparin. Vehicle (n=8), Factor IXai (300 μg/kg, n=8), or heparin (50 U/kg, n=4, or 100 U/kg, n=3) was administered to mice as an intravenous bolus, plasma was obtained at various time points after administration, and the time to clot formation was measured in the modified cephalin clotting time assay described in the Methods section. Relative time to clot formation was determined as the ratio of the time to clot formation of a given experimental condition relative to the mean time to clot formation of vehicle-treated controls. Means±SEM are shown. 15b. Dose-dependent antithrombotic effects of Factor IXai. Mice were given an intravenous single bolus of either vehicle (n=8) or Factor IXai at doses of 150 μg/kg (n=4), 300 μg/kg (n=8), 600 μg/kg (n=4), or 1200 μg/kg (n=4), and plasma was obtained at 45 minutes after administration. Time to clot formation was measured and data expressed as in the a panel. *p<0.05, ***p<0.001 Vs vehicle-treated animals.

FIG. 16. Effect of Factor IXai on fibrin deposition and platelet accumulation

(A) Fibrin deposition was quantified by immunoblotting plasmin digests of pulmonary tissue. In these immunoblots, fibrin was judged to be present in the central band (the one of greatest intensity) which corresponds in molecular weight to the single band detected when fibrin prepared in vitro was used as a positive control. As a negative control, mouse fibrinogen from company in itself was loaded. In these studies, lungs subjected to ischemia and reperfusion exhibited markedly increased fibrin accumulation (3.6-fold by densitometry) compared with that detected in fresh lung tissue. Because this density of this band showed the amount of fibrin in the same volume of protein. Low dose of heparin obviously expressed most fibrin deposition in the lungs exposed to ischemia/reperfusion of all groups except control (R-3). Less fibrin deposition of factor IXai and heparin depends on the higher dose.

(B) As a more rapid and semiquantitative assay for platelet deposition, ¹¹¹In-labeled platelets were injected immediately before reperfusion, after which the lung was reperfused for three hours and then excised and the relative accumulation of radiolabelled platelets in the postischemic lung quantified. At intermediate doses of heparin and Factor IXai, only the Factor IXai was associated with a decrease in the relative accumualtion of platelets in the lung, althoguh there was a trend in that direction in the heparin group as well. N=5 for each group; Means±SEM are shown; *=p<0.05 vs R-3.

(C) Immunostaining for fibrin in the left lung exposed to ischemia/reperfusion with or without heparin and active-site blockade factor IXa. Intravascular fibrin formation can be seen as red staining in the post-ischemic and reperfused lung. When heparin and Factor IXai was administered at intermediate dose immediately before reperfusion, there are no apparent fibrin deposition in the vessels.

FIG. 17. Effect of Factor IXai on survival in murine ischemia/reperfusion model

To measure the pulmonary function of the lung subjected to ischemia/reperfusion, the contralateral (previously nonmanipulated) right lung was physically excluded from the circulation at the termination of the three hour left lung reperfusion period. Survival of the animal then depended entirely upon the function of the postischemic left lung. At the prespecified thirty minute time point following exclusion of the right lung from the circulation, treatment with heparin at any dose was observed to have no effect on survival compared with vehicle treated controls, in contrast, mice treated with an intermediate dose (300 μg/kg) of Factor IXai exhibited a much higher rate of survival. Although the highest dose of Factor IXai showed no effect on survival, there was a tendency (P=0.098) for animals pretrerated with 150 μg/kg of Factor IXai to have improved survival. N=12 for each group; Means±SEM are shown; *=p<0.05 vs R-3.

FIG. 18. Effect of IXai on perioperative blood loss and picture of the gauze

(A) The degree of surgical blood loss with both therapies was objectively quantified. Two gauze pads were placed in a standardized way over the surgical wound after hemostasis was initially achieved under visual inspection. After 4 hours (1 hr ischemia+3 hrs reperfusion), the gauze pads were removed and their hemoglobin content was quantified. These data showed the expected result, in that the least amount of surgical bleeding was detected in the nonanticoagulated animals, whereas there was a progressive increase in the amount of surgical blood loss with increasing doses of heparin. Although at the highest dose of Factor IXai tested (600 μg/kg), there was also an increase in surgical bleeding, the two lower doses (including the 300 μg/kg dose which was functionally beneficial) did not result in an increase in surgical blood loss. N=4 for each group; Means±SEM are shown; *=p<0.05 vs R-3.

(B) These data of perioperative blood loss are graphically illustrated by the appearance of representative blood-soaked gauze pads from the surgical wound. The blood on the gauze in the heparin high dose group demonstrated clealy much more volume than other groups. The blood loss on the gauze depends on the dose of heparin and Factor IXai, however, blood loss in Factor IXai at the effective dose is almost similar to that in the control R-3 group.

FIG. 19. Eeffect of IXai on hemorrhage in lung tissue: A spectrophotometric assay for hemoglobin was used to detect residual hemoglobin after flushing the lungs with saline prior to harvest. Although in general, most experimental conditions revealed similar levels of residual hemoglobin content, mice pretreated with 600 U/kg of heparin demonstrated a significant increase in intraparenchymal hemorrhage compared with the other groups. This, in addition to the excessive blood loss in the surgical wound itself, may have detracted from what may have otherwise been protective effects due to its antithrombotic actions. Note that there was no increase in intraparenchymal pulmonary hemorrhage at any of the tested doses of Factor IXai. N=4 for each group; Means±SEM are shown; *=p<0.05 vs fresh.

FIG. 20. Difference of Bleeding time by using Factor IXai and heparin

Bleeding time were measured in mice that were not subjected to experimental manupulation other than by receiving vehecle, heparin, and IXai prepared in physiological saline and administrated intravenously 5 min before the experiment. After anesthesia, a standardized incision was made on the central tail vein, and the tail was then immersed in physiological saline at 37.5° C. Time was recorded from the moment blood was observed to emerge from the wound until cessation of blood flow. The therapeutically effective dose of Factor IXai (300 μg/kg) does not increase the tail vein bleeding time, although heparin at an intermediate dose does increase the tail vein bleeding time N=4 for each group; Means±SEM are shown; *=p<0.05 vs fresh.

FIG. 21. Effect of Factor IXai on cytokine level: We measured the cytokine levels of IL-1α, IL-1β, TNFα, IL-6, IL-10 in plasma samples by the method of ELISA, only IL-1β demonstrated significant different between IX ai and Heparin groups (2.57±0.47, 5.57+1.41, p<0.05, respectively). But there were no significant differentiation in other cytokine levels. N=S for each group; Means±SEM are shown; *=p<0.05 vs fresh

FIG. 22. Procedure for taking tissue and blood samples

### DETAILED DESCRIPTION OF THE INVENTION

The present invention provides a method for treating an ischemic disorder in a subject which comprises administering to the subject a pharmaceutically acceptable form of a Factor IXa compound in a sufficient amount over a sufficient period of time to inhibit coagulation so as to treat the ischemic disorder in the subject.

The present invention also provides a method for treating an ischemic disorder in a subject which comprises administering to the subject a pharmaceutically acceptable form of a Factor IXa compound in a sufficient amount over a sufficient period of time to inhibit coagulation so as to treat the ischemic disorder in the subject.

The present invention provides a method for treating an ischemic disorder in a subject which comprises administering to the subject a pharmaceutically acceptable form of a Factor IXa compound and a pharmaceutically acceptable form of an indirect or direct fibrinolytic agent, each in a sufficient amount over a sufficient period of time to inhibit coagulation so as to treat the ischemic disorder in the subject.

In another embodiment, the ischemic disorder comprises a peripheral vascular disorder, a pulmonary embolus, a venous thrombosis, a myocardial infarction, a transient ischemic attack, unstable angina, a reversible ischemic neurological deficit, sickle cell anemia or a stroke disorder.

In another embodiment, the ischemic disorder is iatogenically induced. In another embodiment, the subject is undergoing angioplasty, heart surgery, lung surgery, spinal surgery, brain surgery, vascular surgery, abdominal surgery, or organ transplantation surgery. In another embodiment, the organ transplantation surgery comprises heart, lung, pancreas or liver transplantation surgery.

In another embodiment, the period of time comprises from about 5 days before surgery or onset of the disorder to about 5 days after surgery or the onset of the disorder. In another embodiment, the period of time comprises from about 1 hour before surgery or the onset of the disorder to about 12 hours after surgery or the onset of the disorder. In another embodiment, the period of time comprises from about 12 hours before surgery or the onset of the disorder to about 1 hour after surgery or the onset of the disorder. In another embodiment, the period of time comprises from about 1 hour before surgery or the onset of the disorder to about 1 hour after surgery or the onset of the disorder.

In one embodiment, the subject is a mammal. In another embodiment, the mammal is a human. In another embodiment, the amount comprises from about 75 μg/kg to about 550 μg/kg.

In another embodiment, the amount comprises 300 μg/kg. In one embodiment, the direct fibrinolytic agent comprises plasmin or viper venom. In another embodiment, the indirect fibrinolytic agent comprises tissue plasminogen activator, urokinase, streptokinase, RETROVASE®, or recombinant tissue plasminogen activator.

The present invention also provides for a method for identifying a compound that is capable of improving an ischemic disorder in a subject which comprises: a) administering the compound to an animal, which animal is a stroke animal model; b) measuring stroke outcome in the animal, and c) comparing the stroke outcome in step (b) with that of the stroke animal model in the absence of the compound so as to identify a compound capable of improving an ischemic disorder in a subject. In another embodiment, the compound is a Factor IXa compound.

In one embodiment, the stroke animal model comprises a murine model of focal cerebral ischemia and reperfusion. In another embodiment, the stroke outcome is measured by physical examination, magnetic resonance imaging, laser doppler flowmetry, triphenyl tetrazolium chloride staining, chemical assessment of neurological deficit, computed tomography scan, or cerebral cortical blood flow.

The present invention provides a method for treating a reperfusion injury in a subject which comprises administering to the subject a Factor IXa compound in a sufficient amount over a sufficient period of time to inhibit coagulation so as to treat the reperfusion injury in the subject. In one embodiment, the Factor IXa compound comprises recombinant inactivated Factor IXa. In another embodiment, the Factor IXa compound is a peptide, a peptidomimetic, a nucleic acid, a small molecule, a mutated peptide or nucleic acid, a mutein, an antibody or fragment thereof. In another embodiment, the Factor IXa compound is a synthetic molecule.

The present invention provides for a proteolytically inactive recombinant mutein of Factor IX, which has substantially the same amino acid sequence as normal Factor IX but which has an amino acid substitution for one or more of His221, Asp269 or Ser365.

In one embodiment, the mutein has a Ser365 to Ala substitution.

The present invention also provides a proteolytically inactive recombinant mutein of Factor IXa which has substantially the same amino acid sequence as normal human Factor IXa but which has an amino acid substitution for one or more of His41, Asp89 or Ser185 in the heavy chain of Factor IXa. In one embodiment, the mutein has a Ser185 to Ala substitution.

In another embodiment, an isolated cDNA encodes the mutein. In another embodiment, a replicable vector comprises the cDNA. In another embodiment, a microorganism is transfected with the vector. In another embodiment, an expression vector comprises DNA which encodes the mutein. In another embodiment, a microorganism is transfected with the vector. In one embodiment, the Factor IXa compound comprises the mutein.

The present invention provides a method of inhibiting clot formation in a subject which comprises adding to blood an amount of an inactive recombinant mutein in an amount effective to inhibit clot formation in the subject but which does not significantly interfere with hemostasis when the blood is administered to a patient. In another embodiment, the patient has experienced an ischemic event.

The present invention provides for an assay to monitor the effect of a Factor IXa compound administered to a subject to treat an ischemic disorder in the subject which comprises: a) measuring the ischemic disorder in the subject; b) administering the Factor IXa compound to the subject and measuring the ischemic disorder, and c) comparing the measurement of the ischemic disorder in step (b) with that measured in step (a) so as to monitor the effect of the Factor IXa compound. In one embodiment, the ischemic disorder is measured by physical examination, magnetic resonance imaging, laser doppler flowmetry, triphenyl tetrazolium chloride staining, chemical assessment of neurological deficit, computed tomography scan, or cerebral cortical blood flow.

As used herein, the “ischemic disorder” encompasses and is not limited to a peripheral vascular disorder, a venous thrombosis, a pulmonary embolus, a myocardial infarction, a transient ischemic attack, lung ischemia, unstable angina, a reversible ischemic neurological deficit, adjunct thromolytic activity, excessive clotting conditions, reperfusion injury, sickle cell anemia, a stroke disorder or an iatrogenically induced ischemic period such as angioplasty.

In one embodiment of the present invention, the subject is undergoing heart surgery, angioplasty, lung surgery, spinal surgery, brain surgery, vascular surgery, abdominal surgery, or organ transplantation surgery. The organ transplantation surgery may include heart, lung, pancreas or liver transplantation surgery.

In the intrinsic pathway, Factor XIa cleaves Factor IX between Arg145 and Ala146 and between Arg 180-Val181, releasing a 35 amino acid peptide and producing Factor IXa having a 145 amino acid light chain (amino acids 1-145) and a 235 amino acid heavy chain (amino acids 181-415) joined by a disulfide bond between cysteine residues at positions 132 and 289. Factor IXa is a serine protease which, when complexed with Factor VIIIa on membrane surfaces, converts Factor X to its active form Factor Xa. The enzyme active site of Factor IXa is located on the heavy chain. Three amino acids in the heavy chain are principally responsible for the catalytic activity, His221, Asp269 and Ser365 (H221, D269 and S365, the catalytic triad). If the amino acids of the heavy chain are numbered from 1 to 235, the catalytic triad is His41, Asp89 and Ser185, and the disulfide bond joining the heavy chain to the light chain is at Cys109 on the heavy chain.

As used herein “a Factor IXa compound” means a compound which inhibits or reduces the conversion of Factor X to Factor Xa by naturally occurring Factor IX. As used herein, a Factor IXa compound may be chosen from one of several subsets. One subset is a chemically modified form of naturally occurring Factor IXa which chemical modification results in the inactivation of Factor IXa (e.g., inactivated Factor IXa, active-site blocked Factor IXa or Factor IXai). Another subset of a Factor IXa compound is any recombinant mutated form of Factor IXa (e.g., a mutein form of Factor IXa, a recombinant Factor IXa with a deletion or Factor IXami). In addition, there are other subsets of a Factor IXa compound which include but are not limited to, for example: (1) nucleic acids, (2) anti-Factor IXa antibodies or fragments thereof, (3) saccharides, (4) ribozymes, (5) small organic molecules, or (6) peptidomimetics.

Thus, a Factor IXa compound may encompass the following: a Glu-Gly-Arg chloromethyl ketone-inactivated human factor IXa, an inactive Christmas factor, a Glu-Aly-Arg chloromethyl ketone-inactivated factor IXa, a glutamyl-glycyl-arginyl-Factor IXa, a dansyl Glu-Gly-Arg chloromethyl ketone-inactivated bovine factor IXa (IXai), a Factor IXai, a competitive inhibitor of Factor IXa, a peptide mimetic of Factor IXa, a carboxylated Christmas factor, a competitive inhibitor of the formation of a Factor IXa/VIIIa/X complex, a des-y-carboxyl Factor IX, Factor IX lacking a calcium-dependent membrane binding function, inactive Factor IX including only amino acids 1-47, apoFactor IX including amino acids 1-47, Factor IX Bm Kiryu, a Val-313-to-Asp substitution in the catalytic domain of Factor IX, a Gly-311-to-Glu substitution in the catalytic domain of Factor IX, a Gly-311 to Arg-318 deletion mutant of Factor IX, an anti-Factor IXa antibody, an anti-Factor IXa monoclonal or polyclonal antibody. The Factor IXa compound may also include inactive species of Factor IX described in the references provided herein, especially Freedman et al., 1995; Furie and Furie, 1995; Miyata et al., 1994 and Wacey et al., 1994. Factor IX or Factor IXa may be obtained from blood.

Thus, a Factor IXa compound may be Factor IXa in which the active site is blocked and may be prepared as described in Experimental Details below. The Factor IXa compound may be a Factor IXa which includes post-translational modifications including glycosylation, β-hydroxylation of aspartic acid, γ-carboxylation of glutamic acid and propeptide cleavage. The Factor IXa compound may be concentrated via heparin affinity chromatography or hydrophobic interaction chromatography. The Factor IXa compound may be a genetically engineered, a recombinant Factor IXa in which amino acids at the active site, especially the serine amino acid at the active site, have been altered to render the recombinant Factor IXa functionally inactive, but still capable of competing with intact, native Factor IXa for cell surface binding. In another embodiment, the Factor IXa compound is a synthetic molecule. In another embodiment, the carrier comprises an aerosol, intravenous, oral or topical carrier.

In one embodiment of the present invention the Factor IXa compound is a form of Factor IXa inactivated by the standard methods known to one of skill in the art, such as mutation of the gene which encodes Factor IXa.

As used herein an “indirect fibrinolytic agent” is an agent whose activity indirectly results in fibrin lysis. In one embodiment, an indirect fibrinolytic agent comprises tissue plasminogen activator (tPA), urokinase, streptokinase, RETROVASE®, or recombinant tissue plasminogen activator. As used herein “direct fibrinolytic agent” is an agent that is capable of fibrinolysis. In one embodiment, a direct fibrinolytic agent is plasmin or viper venom. In one embodiment, the amount of fibrinolytic agent administered to a subject is up to the amount necessary to lyse an intravascular fibrin clot or an amount to cause lysis of a formed intravascular fibrin clot.

One embodiment of the present invention is wherein the Factor IXa compound is inactivated by the standard methods known to one of skill in the art, such as mutation. Factor IXa compound may be an antagonist of Factor IXa. Such antagonist may be a peptide mimetic, a nucleic acid molecule, a ribozyme, a polypeptide, a small molecule, a carbohydrate molecule, a monosaccharide, an oligosaccharide or an antibody.

A preferred embodiment of the present invention is wherein the Factor IXa compound is an active site-blocked Factor IXa or a Glu-Gly-Arg chloromethyl ketone-inactivated human factor IXa. In a preferred embodiment, the effective amount is from about 0.1 μg/ml plasma to about 250 μg/ml plasma or from about 0.5 μg/ml plasma to about 25 μg/ml plasma or preferably from 0.7 μg/ml plasma to about 5 μg/ml plasma.

Another embodiment of present invention is where the sufficient amount includes but is not limited to from about 75 μg/kg to about 550 μg/kg. The amount may be 300 μg/kg.

In an embodiment of the present invention the Factor IXa compound is an inactive mutein form of Factor IXa which is useful as selective antithrombotic agent. As used herein, “mutein form” of Factor IXa means a protein which differs from natural factor IXa by the presence of one or more amino acid additions, deletions, or substitutions which reduce or eliminate the ability of the protein to participate in the conversion of Factor X to Factor Xa.

In another embodiment of the present invention the Factor IXa compound is a proteolytically inactive, recombinant mutein form of Factor IX, which has substantially the same amino acid sequence as normal or native human Factor IX but in which a different amino acid has been substituted for one or more of His221, Asp269 and Ser365. The present invention also provides a proteolytically inactive, recombinant mutein form of Factor IXa, which has substantially the same amino acid sequence as normal or native human factor IXa but in which a different amino acid has been substituted for one or more of His41, Asp89 or Ser185 in the heavy chain of Factor IXa. The term “proteolytically inactive” means that the muteins are incapable of converting Factor X to Factor Xa.

The invention also provides a method of inhibiting thrombosis in a human patient which comprises administering to the patient, or adding to the blood which is to be administered to the patient, an amount of an inactive recombinant mutein of this invention which is effective to inhibit thrombosis but which does not significantly interfere with hemostasis in the patient.

Recombinant muteins of Factor IX useful in this invention are referred to collectively as Factor Ixmi (i.e., Factor IX mutationally inactivated). Recombinant muteins of Factor IXa useful in this invention are referred to collectively as Factor IXami. Examples of Factor IXa compounds which are recombinant muteins are as follows:
Factor IXmi (Ser365-Xxx)
Factor IXmi (Asp269-Yyy)
Factor IXmi (His221-Zzz)
Factor IXmi (Ser365-Xxx, Asp269-Yyy)
Factor IXmi (Ser 365-Xxx, His221-Zzz)
Factor IXmi (Asp269-Yyy, His-Zzz)
Factor IXmi (Ser365-Xxx, Asp269-Yyy, His-Zzz)
Factor IXami (Ser365-Xxx)
Factor IXami (Asp269-Yyy)
Factor IXami (His221-Zzz)
Factor IXami (Ser365-Xxx, Asp269-Yyy)
Factor IXami (Ser365-Xxx, His221-Zzz)
Factor IXami (Asp269-Yyy, His-Zzz)
Factor IXami (Ser365-Xxx, Asp269-Yyy, His-Zzz)
   wherein Xxx is any one of the standard amino acids other than serine, Yyy is any one of the standard amino acids other than aspartic acid, and Zzz is any of the standard amino acids other than histidine. Preferred recombinant muteins are Factor IXmi(Ser365-Ala) and Factor IXami (Ser365-Ala).

Factor IXmi and Factor IXami are functionally similar to Factor IXai in terms of their ability to establish effective anti-coagulation intravascularly and in ex vivo equipment connected to the blood stream while permitting retention of effective hemostasis. The advantages of Factor IXmi and Factor IXami over Factor IXai are the following:
Factor IXmi and Factor IXami can be produced directly in a genetically engineered organism, thus avoiding several processing and purification steps with their attendant losses, thereby improving yield of product.
The cost of production of Factor IXmi and Factor IXami in an appropriate genetically engineered organism is lower than the cost of production of Factor IXai from human plasma.
Factor IXmi and Factor IXami, produced in a genetically engineered organism, will not be subject to the risk of contamination with various infectious agents such as viruses or prions (for example the agents for HIV disease and for bovine and/or human spongiform encephalopathies).
Factor IXmi and Factor IXami, being less different from wild-type human Factor IX and Factor IXa than is the chemically modified Factor IXai, will have a lower probability of eliciting an immune response in patients who are dosed with the modified protein for extended periods of time, thereby reducing the risk of delayed type hypersensitivity reactions and improving the safety for indications such as anticoagulation in hemodialysis that will require repeated, long-term use. The recombinant muteins of this invention can be produced by known genetic engineering techniques, using as starting material recombinant cDNA for Factor IX in an appropriate cloning vector. For example, starting materials which may be used in the production of a Factor IXa compound may be the product of Example 5 of U.S. Pat. No. 4,770,9990 which are recombinant plaques of E. coli infected with bacteriophage M12 mp11 Pst vector containing the entire sequence of recombinant Factor IX cDNA ligated to Pst adapters. The recombinant plaques are used to prepare single-stranded DNA by either the small-scale or large-scale method described in Sambrook et al., Molecular Cloning. A Laboratory Manual. Second Edition, Cold Spring Harbor Press, 1989, pages 4.29-4.30 and 4.32.

The single-stranded. M13 mp11 containing Factor IX cDNA is then used to carry out oligonucleotide-mediated mutagenesis using the double primer method of Zoller and Smith as described in Sambrook et al., 1989, pages 15.51-15.73. Mutagenic primers which can be used include the following:
1) Oligonucleotides for producing Factor IXmi(Ser365-Xxx)
   3′-W ACA GTT CCT CTA XXX CCC CCT GGG GTA V-5′
   where
   W is T, 3′-GT or 3′-AGT
   V is C, 3′-CA, or 3′-CAA
   XXX is the complement to a DNA codon for any one of the standard amino acids other than serine.
2) Oligonucleotides for producing FACTOR IXmi (Asp269-Yyy)
   3′-W TTC ATG TTA GTA YYY TAA CGC GAA GAC V-5′
   where
   W IS A, 3′=TA, OR 3′-TTA
   V is C, 3′-CT, or 3′-CTT
   YYY is the complement to a DNA codon for any one of the standard amino acids other than aspartic acid and cysteine.
3) oligonucleotides for producing Factor IXmi (His221-Zzz)
   3′-TTA CAT TGA CGA CGG ZZZ ACA CAA CTT TGA CCA-5′
   where
   W is A, 3′-AA, or 3′-TAA.
   V is C, 3′-CC, or 3′-CCA
   ZZZ is the complement to a DNA codon for any one of the standard amino acids other than histidine and cysteine.

Oligonucleotide primers for producing the preferred Factor IXmi of this invention, Factor IXmi (Ser365-Ala), are those of No. 1 above, wherein XXX is the complement of a codon for alanine, i.e., 3′-CGA, 3′-CGC, 3′-CGT or 3′-CGC. A specific primer for producing Factor IXmi (Ser365-Ala) is:

3′-GT ACA GTT CCT CTA CGA CCC CCT GGG GTA C-5′

A skilled artisan would recognize and know how to carry out the remaining steps of oligonucleotide-mediated mutagenesis as follows:
Hybridization of mutagenic oligonucleotides to the target DNA.
Extension of the hybridized oligonucleotides to the target DNA.
Transfection of susceptible bacteria.
Screening of plaques for the desired mutation.
Preparation of single-stranded DNA from a mutant plaque.
Sequencing the single-stranded DNA.
Recovery of double-stranded Factor IXmi cDNA.
Inserting the double-stranded Factor IXmi cDNA into the expression vector used by Kaufman (for example)
Expression of Factor IXmi.
Treating the Factor IXmi with Factor XIa to produce Factor IXami.

Another embodiment of the present invention wherein the Factor IXa compound is capable of inhibiting the active site of Factor IXa. Such a compound is obtainable from the methods described herein. The Factor IXa compound may be a peptide, a peptidomimetic, a nucleic acid or a small molecule. The agent may be an antibody or portion thereof. The antibody may be a monoclonal antibody or a polyclonal antibody. The portion of the antibody may include a Fab.

One embodiment of the present invention is wherein the Factor IXa compound is a peptidomimetic having the biological activity of a Factor IXa or a Glu-Gly-Arg chloromethyl ketone-inactivated human Factor IXa wherein the compound has a bond, a peptide backbone or an amino acid component replaced with a suitable mimic. Examples of unnatural amino acids which may be suitable amino acid mimics include α-alanine, L-α-amino butyric acid, L-γ-amino butyric acid, L-α-amino isobutyric acid, L-ε-amino caproic acid, 7-amino heptanoic acid, L-aspartic acid, L-glutamic acid, cysteine (acetamindomethyl), N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, Boc-L-thioproline. (Blondelle, et al. 1994; Pinilla, et al. 1995).

The present invention incorporates U.S. Pat. Nos. 5,446,128, 5,422,426 and 5,440,013 in their entireties as references which disclose the synthesis of peptidomimetic compounds and methods related thereto. The compounds of the present invention may be synthesized using these methods. The present invention provides for peptidomimetic compounds which have substantially the same three-dimensional structure as those compounds described herein.

In addition to the compounds disclosed herein having naturally-occurring amino acids with peptide or unnatural linkages, the present invention also provides for other structurally similar compounds such as polypeptide analogs with unnatural amino acids in the compound. Such compounds may be readily synthesized on a peptide synthesizer available from vendors such as Applied Biosystems, Dupont and Millipore.

Another embodiment of the present invention is a pharmaceutical composition which may include an effective amount of a Factor IXa compound and a pharmaceutically acceptable carrier. The carrier may include a diluent. Further, the carrier may include an appropriate adjuvant, a herpes virus, an attenuated virus, a liposome, a microencapsule, a polymer encapsulated cell or a retroviral vector. The carrier may include an aerosol, intravenous, oral or topical carrier.

The present invention provides for a method for identifying a compound that is capable of improving an ischemic disorder in a subject which includes: a) administering the compound to an animal, which animal is a stroke animal model; b) measuring-stroke outcome in the animal, and c) comparing the stroke outcome in step (b) with that of the stroke animal model in the absence of the compound so as to identify a compound capable of improving an ischemic disorder in a subject. The stroke animal model includes a murine model of focal cerebral ischemia and reperfusion. The stroke outcome may be measured by physical examination, magnetic resonance imaging, laser doppler flowmetry, triphenyl tetrazolium chloride staining, clinical assessment of neurological deficit, computed tomography scan, or cerebral cortical blood flow. The stroke outcome in a human may be measured also by clinical measurements, quality of life scores and neuropsychometric testing.

The present invention provides for treatment of ischemic disorders by inhibiting the ability of the neutrophil, monocyte or other white blood cell to adhere properly. This may be accomplished removing the counter ligand, such as CD18. It has been demonstrated as discussed hereinbelow, that “knock-out” CD18 mice (mice that do not have expression of the normal CD18 gene) are protected from adverse ischemic conditions. The endothelial cells on the surface of the vessels in the subject may also be a target for treatment. In a mouse model of stroke, administration of TPA as a thrombolytic agent caused some visible hemorrhaging along with improvement of the stroke disorder. The present invention may be used in conjunction with a thrombolytic therapy to increase efficacy of such therapy or to enable lower doses of such therapy to be administered to the subject so as to reduce side effects of the thrombolytic therapy.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

This invention also provides for pharmaceutical compositions including therapeutically effective amounts of protein compositions and compounds capable of treating ischemic disorder or improving stroke outcome in the subject of the invention together with suitable diluents, preservatives, solubilizers, emulsifiers, adjuvants and/or carriers useful in treatment of neuronal degradation due to aging, a learning disability, or a neurological disorder. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol) covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound capable of alleviating the symptoms of the stroke disorder or improving the stroke outcome in the subject. Controlled or sustained release compositions include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

Portions of the compound of the invention may be “labeled” by association with a detectable marker substance (e.g., radiolabeled with ¹²⁵I or biotinylated) to provide reagents useful in detection and quantification of compound or its receptor bearing cells or its derivatives in solid tissue and fluid samples such as blood, cerebral spinal fluid or urine.

When administered, compounds are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Attachment of polyethylene glycol (PEG) to compounds is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous compounds. For example, a PEG adduct of a human protein might be useful for the treatment of disease in other mammalian species without the risk of triggering a severe immune response. The compound of the present invention capable of alleviating symptoms of a cognitive disorder of memory or learning may be delivered in a microencapsulation device so as to reduce or prevent an host immune response against the compound or against cells which may produce the compound. The compound of the present invention may also be delivered microencapsulated in a membrane, such as a liposome.

Polymers such as PEG may be conveniently attached to one or more reactive amino acid residues in a protein such as the alpha-amino group of the amino terminal amino acid, the epsilon amino groups of lysine side chains, the sulfhydryl groups of cysteine side chains, the carboxyl groups of aspartyl and glutamyl side chains, the alpha-carboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

By means of well-known techniques such as titration and by taking into account the observed pharmacokinetic characteristics of the agent in the individual subject, one of skill in the art can determine an appropriate dosing regimen. See, for example, Benet, et al., “Clinical Pharmacokinetics” in ch. 1 (pp. 20-32) of Goodman and Gilman's The Pharmacological Basis of Therapeutics, 8th edition, A. G. Gilman, et al. eds. (Pergamon, N.Y. 1990). The present invention provides for a pharmaceutical composition which comprises an agent capable of treating an ischemic disorder or improving stroke outcome and a pharmaceutically acceptable carrier. The carrier may include but is not limited to a diluent, an aerosol, a topical carrier, an aqueous solution, a nonaqueous solution or a solid carrier.

This invention is illustrated in the Experimental Detail section which follows. These sections are set forth to aid in an understanding of the invention but are not intended to, and should not be construed to, limit in any way the invention as set forth in the claims which follow thereafter.

Experimental Details

Abbreviations: EC, endothelial cell; PMN, polymorphonuclear leukocyte; WP, Weibel-Palade body; vWF, von Willebrano factor; EGTA, ethyleneglycol bis (aminoethylether) tetraacetic acid; HBSS, Hank's balanced salt solution; CS, coronary sinus; IL, interleukin; PAF, platelet activating factor; HUVEC, human umbilical vein EC; LR, lactated Ringer's solution; MCAO, middle cerebral artery occlusion; rt-PA, recombinant tissue plasminogen activator; ICH, intracerebral hemorrhage; OD, optical density; MCA, middle cerebral artery; rt-PA, recombinant tissue-type plasminogen activator; TIA, transient ischemic attack; TTC, triphenyltetrazolium chloride.

### EXAMPLE 1

### Procedural and Strain-Related Variables Significantly Effect Outcome in a Murine Model of Focal Cerebral Ischemia

The recent availability of transgenic mice has led to a burgeoning number of reports describing the effects of specific gene products on the pathophysiology of stroke. Although focal cerebral ischemia models in rats have been well-described, descriptions of a murine model of middle cerebral artery occlusion are scant, and sources of potential experimental variability remain undefined. It was hypothesized that slight technical modifications would result in widely discrepant results in a murine model of stroke, and that controlling surgical and procedural conditions could lead to reproducible physiologic and anatomic stroke outcomes. To test this hypothesis, a murine model was established which would permit either permanent or transient focal cerebral ischemia by intraluminal occlusion of the middle cerebral artery (MCA) This study provides a detailed description of the surgical technique, and reveals important differences between strains commonly used in the production of transgenic mice. In addition to strain-related differences, infarct volume, neurologic outcome, and cerebral blood flow appear to be importantly affected by temperature during the ischemic and post-ischemic periods, mouse size, and size of the suture which obstructs the vascular lumen. When these variables were kept constant, there was remarkable uniformity of stroke outcome. These data emphasize the protective effects of hypothermia in stroke, and should help to standardize techniques among different laboratories to provide a cohesive framework for evaluating the results of future studies in transgenic animals.

Introduction:

The recent advent of genetically altered mice provides a unique opportunity to evaluate the role of single gene products in the pathophysiology of stroke. Although there is an increasing number of reports about the effect of cerebral ischemia in transgenic mice, to date, there exists no detailed description of the murine models involved, nor is there a detailed analysis of potentially important procedural variables which may effect stroke outcome, Most descriptions of a murine model (1,4,8,9,14,17-19,23,24; see references listed at end of Example 1) are devolved descriptions of the widely used rat models of focal cerebral ischemia (22,26). Although there has been some attention paid to strain related differences in the susceptibility of mice to cerebral ischemia (4), few technical considerations have been addressed in published studies. Because pilot data demonstrated that minor differences in operative procedure or postoperative care translated into major differences in stroke outcome, the current study was undertaken to systematically identify important surgical, technical, and anatomic considerations required to obtain consistent results in a murine model of focal cerebral ischemia. When strokes are created in a rigidly controlled manner, differences, due to the absence (or overexpression) of a single gene product, should be readily discernable.

This study presents a detailed rendering of a reproducible murine model of focal cerebral infarction based on modifications of the original rat model (26) This study identifies procedural variables that have a large impact on stroke outcome which have not been previously reported in technical descriptions of murine stroke models. These variables include suture length and gauge, methods of vascular control, temperature regulation in mice, and differences between strains commonly used in the breeding of transgenic animals. As the model described lends itself to the study of either permanent or transient focal cerebral ischemia, evidence is presented that with carefully chosen ischemia times, infarct volume and mortality in reperfused animals can be made to approximate those seen with permanent occlusion. Understanding potential model-dependent sources of variability in stroke outcome can help to clarify divergent results between different laboratories. Adoption of a standardized model which yields consistent results is an important first step towards the use of transgenic mice in the study of the pathophysiology of stroke.

Materials and Methods:

Animal Purchase and Anesthesia: Male mice of three different strains (C57 BlackJ6, CD-1 and 129J) were purchased from Jackson Laboratories (Bar Harbor, Me.). Animals were eight to ten weeks of age and weighed between 18-37 grams (as indicated) at the time of experiments. Mice were anesthetized with an intraperitoneal injection of 0.3 ml of ketamine (10 mg/cc) and xylazine (0.5 mg/cc). An additional dose of 0.1 cc was given prior to withdrawal of the catheter in animals undergoing transient ischemia. On the day following surgery, anesthesia was repeated immediately prior to laser doppler flow measurement and humane euthansia. These procedures have been approved by the Institutional Animal Care and Use Committee at Columbia University, and are in accordance with AALAC guidelines for the humane care and use of laboratory animals.

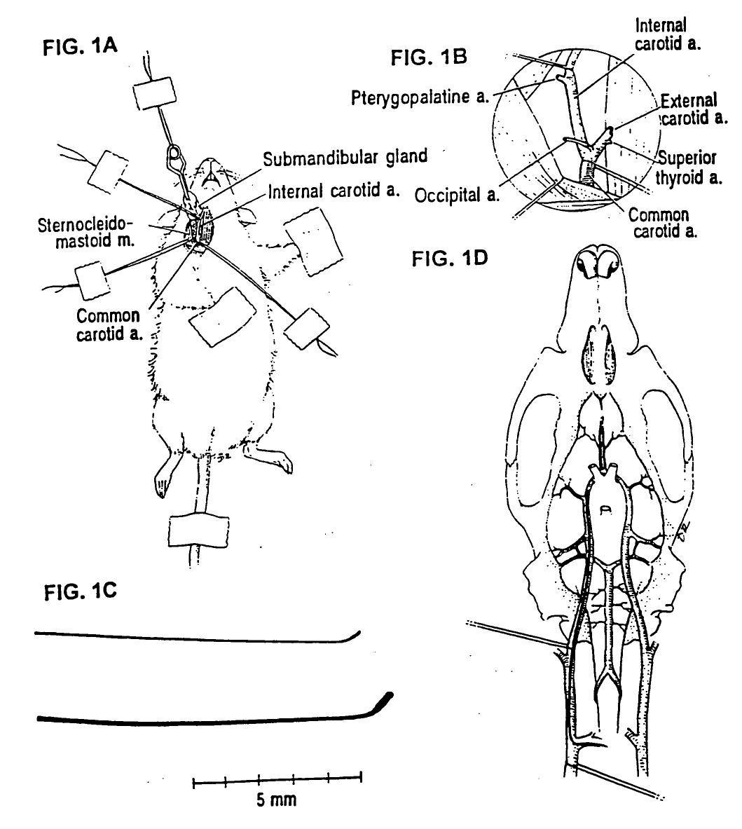

Surgical Set-up: The animal was positioned supine on a gauze pad which rests on a temperature controlled operating surface (Yellow Springs Instruments, Inc. [YSI], Yellow Springs, Ohio). A rectal temperature probe (YSI) was inserted, in order to regulate the temperature of the operating surface to maintain a constant animal core temperature of 36-38° C. To facilitate exposure, the right hindpaw and left forepaw were taped to the operating surface, the right forepaw was taped to the animal's chest, and the tail was taped to the rectal probe (FIG. 1A). A midline neck incision was made by gently lifting the loose skin between the manubrium and the jaw and excising a 1 cm² circle of skin. The paired midline submandibular glands directly underlying this area were bluntly divided, with the left gland left in situ. The right gland was retracted cranially with an small straight Sugita aneurysm clip (Mizutto America, Inc., Beverly, Mass.) secured to the table by a 4.0 silk and tape. The sternocleidomastoid muscle was then identified, and a 4.0 silk ligature placed around its belly. This ligature was drawn inferolaterally, and taped to the table, to expose the omohyoid muscle covering the carotid sheath. The exposure is shown in FIG. 1B.

Operative Approach: Once the carotid sheath was exposed, the mouse and the temperature control surface were placed under an operating microscope (16-25× zoom, Zeiss, Thornwood, N.Y.), with a coaxial light source used to illuminate the field. Under magnification, the omohyoid muscle was carefully divided with pickups. The common carotid artery (CCA) was carefully freed from its sheath, taking care not to apply tension to the vagus nerve (which runs lateral to the CCA). Once freed, the CCA was isolated with a 4.0 silk, taped loosely to the operating table. Once proximal control of the CCA was obtained, the carotid bifurcation was placed in view. The occipital artery, which arises from the proximal external carotid artery and courses postero-laterally across the proximal internal carotid artery (ICA) to enter the digastric muscle, was isolated at its origin, and divided using a Malis bipolar microcoagulator (Codman-Schurtleff, Randolph, Mass.). This enabled better visualization of the ICA as it courses posteriorly and cephalad underneath the stylohyoid muscle towards the skull base. Just before the ICA enters the skull it gives off a pterygopalatine branch, which courses laterally and cranially. This branch was identified, isolated, and divided at its origin, during which time the CCA-ICA axis straightens. A 4.0 silk suture was then placed around the internal carotid artery for distal control, the end of which was loosely taped to the operating surface.

Next, the external carotid artery was placed in view. Its cranio-medial course was skeletonized and its first branch, the superior thyroid artery, was cauterized and divided. Skeletonization was subsequently carried out distally by elevation of the hyoid bone to expose the artery's bifurcation into the lingual and maxillary arteries. Just proximal to this bifurcation the external carotid was cauterized and divided. Sufficient tension was then applied to the silk sutures surrounding the proximal common, and distal internal, carotid arteries to occlude blood flow, with care taken not to traumatize the arterial wall. Tape on the occluding sutures was readjusted to maintain occlusion.

Introduction and Threading of the Occluding Intraluminal Suture: Immediately following carotid occlusion, an arteriotomy was fashioned in the distal external carotid wall just proximal to the cauterized area. Through this arteriotomy, a heat-blunted 5.0 or 6.0 nylon suture (as indicated in the Results section) was introduced (FIGS. 1C and 1D). As the suture was advanced to the level of the carotid bifurcation, the external stump was gently retracted caudally directing the tip of the suture into the proximal ICA. Once the occluding suture entered the ICA, tension on the proximal and distal control sutures was relaxed, and the occluding suture was slowly advanced up the ICA towards the skull base under direct visualization (beyond the level of the skull base, sight of the occluding suture is lost). Localization of the distal tip of the occluding suture across the origin of the middle cerebral artery (MCA) (proximal to the origin of the anterior cerebral artery) was determined by the length of suture chosen (12 mm or 13 mm as indicated in the Results section, shown in FIG. 1C), by laser doppler flowmetry (see Ancillary physiological procedures section), and by post-sacrifice staining of the cerbral vasculature (see below). After placement of the occluding suture was complete, the external carotid artery stump was cauterized to prevent bleeding through the arteriotomy once arterial flow was reestablished.

Completion of Surgical Procedure: For all of the experiments shown, the duration of carotid occlusion was less than two minutes. To close the incision, the sutures surrounding the proximal and distal CCA, as well as the sternocleidomastoid muscle, were cut and withdrawn. The aneurysm clip was removed from the submandibular gland and the gland was laid over the operative field. The skin edges were then approximated with one surgical staple and the animal removed from the table.

Removal of the Occluding Suture to Establish Transient Cerebral Ischemia: Transient cerebral ischemia experiments required reexploration of the wound to remove the occluding suture. For these experiments, initial wound closure was performed with a temporary aneurysm clip rather than a surgical staple to provide quick access to the carotid. Proximal control with a 4-0 silk suture was reestablished prior to removal of the occluding suture to minimize bleeding from the external carotid stump. During removal of the occluding suture, cautery of the external carotid artery stump was begun early, before the distal suture has completely cleared the stump. Once the suture was completely removed, the stump is more extensively cauterized. Reestablishment of flow in the extracranial internal carotid artery was confirmed visually and the wound was closed as for permanent focal ischemia described above. Confirmation of intracranial reperfusion was accomplished with laser doppler flowmetry (see Ancillary physiological procedures section).

Calculation of Stroke Volume: Twenty-four hours after middle cerebral artery occlusion, surviving mice were reanesthetized with 0.3 cc of ketamine (10 mg/ml) and xylazine (0.5 mg/ml). After final weights, temperatures and cerebral blood flow readings were taken (as described below), animals were perfused with 5 ml of a 0.15% solution of methylene blue and saline to enhance visualization of the cerebral arteries. Animals were then decapitated, and the brains were removed. Brains were then inspected for evidence of correct catheter placement, as evidenced by negative staining of the vascular territory subtended by the MCA, and placed in a mouse brain matrix (Activational Systems Inc., Warren, Mich.) for 1 mm sectioning. Sections were immersed in 2% 2,3,5-triphenyltetrazolium chloride (TTC) in 0.9% phosphate-buffered saline, incubated for 30 minutes at 37° C., and placed in 10% formalin (5). After TTC staining, infarcted brain was visualized as an area of unstained (white) tissue in a surrounding background of viable (brick red) tissue. Serial sections were photographed and projected on tracing paper at a uniform magnification; all serial sections were traced, cut out, and the paper weighed by a technician blinded to the experimental conditions. Under these conditions, infarct volumes are proportional to the summed weights of the papers circumscribing the infarcted region, and were expressed as a percentage of the right hemispheric volume. These methods have been validated in previous studies (3,12,15,16).

Ancillary Physiological Studies:

Ancillary physiogical studies were performed on each of the three different strains used in the current experiments, immediately prior to and after the operative procedure. Systemic blood pressures were obtained by catheterization of the infrarenal abdominal aorta, and measured using a Grass Model 7 polygraph (Grass Instrument Co., Quincy, Mass.). An arterial blood sample was obtained from this infrarenal aortic catheter; arterial pH, pCO, (mm Hg). PO (mm HQ) and hemoolobin oxygen saturation (%) were measured using a Blood Gas Analyser and Hemoglobinometer (Grass Instrument Co., Quincy, Mass.). Because of the need for arterial puncture and abdominal manipulation to measure these physiologic parameters, animals were designated solely for these measurements (stroke volumes, neurologic outcome, and cerebral blood flows were not measured in these same animals).

Transcranial measurements of cerebral blood flow were made using laser doppler flowmetry (Perimed, Inc., Piscataway, N.J.) after reflection of the skin overlying the calvarium, as previously described (10) (transcranial readings were consistently the same as those made after craniectomy in pilot studies). To accomplish these measurements, animals were placed in a stereotactic head frame, after which they underwent midline skin incision from the nasion to the superior nuchal line. The skin was swept laterally, and a 0.7 mm straight laser doppler probe (model #PF2B) was lowered onto the cortical surface, wetted with a small amount of physiologic saline. Readings were obtained 2 mm posterior to the bregma, both 3 mm and 6 mm to each side of midline using a sterotactic micromanipulator, keeping the angle of the probe perpendicular to the cortical surface. Relative cerebral blood flow measurements were made immediately after anesthesia, after occlusion of the MCA, and immediately prior to euthanasia, and are expressed as the ratio of the doppler signal intensity of the ischemic compared with the nonischemic hemisphere. For animals subjected to transient cerebral ischemia, additional measurements were made just before and just after withdrawal of the suture, initiating reperfusion.

The surgical procedure/intraluminal MCA occlusion was considered to be technically adequate if ≧50% reduction in relative cerebral blood flow was observed immediately following placement of the intraluminal occluding catheter (15 of the 142 animals used in this study [10.6%] were exluded due to inadequate drop in blood flow at the time of occlusion). These exclusion criteria were shown in preliminary studies to yield levels of ischemia sufficient to render consistent infarct volumes by TTC staining. Reperfusion was considered to be technically adequate if cerebral blood flow at catheter withdrawal was at least twice occlusion cerebral blood flow (13/17 animals in this study [76%]).

Temperature: Core temperature during the peri-infarct period was carefully controlled throughout the experimental period. Prior to surgery, a baseline rectal temperature was recorded (YSI Model 74 Thermistemp rectal probe, Yellow Springs Instruments, Inc., Yellow Springs, Ohio). Intraoperatively, temperature was controlled using a thermocouple-controlled operating surface. Following MCA occlusion, animals were placed for 90 minutes in an incubator, with animal temperature maintained at 37° C. using the rectal probe connected via thermocouple to a heating source in the incubator. Temperature was similarly controlled in those animals subjected to transient ischemia, including a 45 minute (ischemic) period as well as a 90 minute post-ischemic period in the incubator. Following placement in the core-temperature incubator, animals were returned to their cages for the remaining duration of pre-sacrifice observation.

Neurological Exam: Prior to giving anesthesia at the time of euthanasia, mice were examined for obvious neurological deficit using a four-tiered grading system: (1) normal spontaneous movements, (2) animal circling towards the right, (3) animal spinning to the right, (4) animal crouched on all fours, unresponsive to noxious stimuli. This system was shown in preliminary studies to accurately predict infarct size, and is based on systems developed for use in rats (6).

Data Analysis: Stroke volumes, neurologic outcome scores, cerebral blood flows and arterial blood gas data were compared using an unpaired Student's t-test. Values are expressed as means±SEM, with a p<0.05 considered statistically significant. Mortality data, where presented was evaluated using chi-squared analysis.

Results:

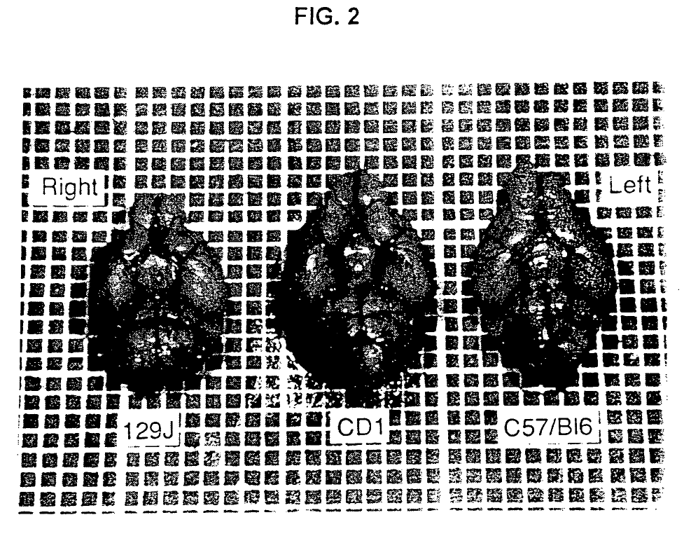

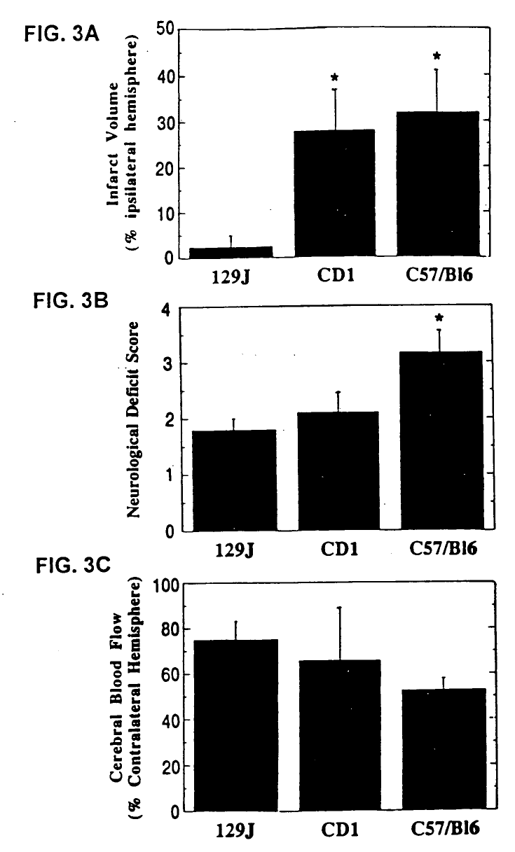

Effects of Strain: Three different commonly used mouse strains (CD1, C57/B16, and 129J) were used to compare the variability in stroke outcome following permanent focal cerebral ischemia. To establish that there were no gross anatomic differences in collateralization of the cerebral circulation, the Circle of Willis was visualized using India ink in all three strains (FIG. 2). These studies failed to reveal any gross anatomic differences. Mice of similar sizes (20±0.8 g, 23±0.4 g, and 23±0.5 g for 129J, CD1, and C57B1 mice, respectively) were then subjected to permanent focal ischemia under normothermic conditions using a 12 mm length of 6-0 nylon occluding suture. Significant strain-related differences in infarct volume were noted, with infarcts in 129J mice being significantly smaller than those observed in CD1 and C57/B16 mice despite identical experimental conditions (FIG. 3A). Differences in infarct size were paralleled by neurological exam, with the highest scores (i.e., most severe neurologic damage) being seen in the C57/B16 and CD1 mice (FIG. 3B).

To determine the relationship between infarct volume and cerebral blood flow to the core region, laser doppler flowmetry was performed through the thin murine calvarium. No preoperative strain-related differences in cerebral blood flow were observed, corresponding to the lack of gross anatomic differences in vascular anatomy (FIG. 2). Measurement of cerebral blood flow immediately following insertion of the occluding catheter revealed that similar degees of flow reduction were created by the procedure (the percentage of ipsilateral/contralateral flow immediately following insertion of the obstructing catheter was 23±2%, 19±2%, 17±3% for 129J, CD1, and C57/B16 mice, respectively). Not surprisingly, blood flow to the core region measured at 24 hours just prior to euthanasia demonstrated the lowest blood flows in those animals with the most severe neurologic injury (FIG. 3C).

Anatomic and Physiologic Characteristics of Mice: Baseline arterial blood pressures, as well as arterial blood pressures following middle cerebral artery occlusion, were nearly identical for all animals studied, and were not effected by mouse strain or size (Table I). Analysis of arterial blood for pH, pCO₂, and hemoglobin oxygen saturation (%) similarly revealed no significant differences (Table I).

Effect of Animal Size and Bore of the Occluding Suture:

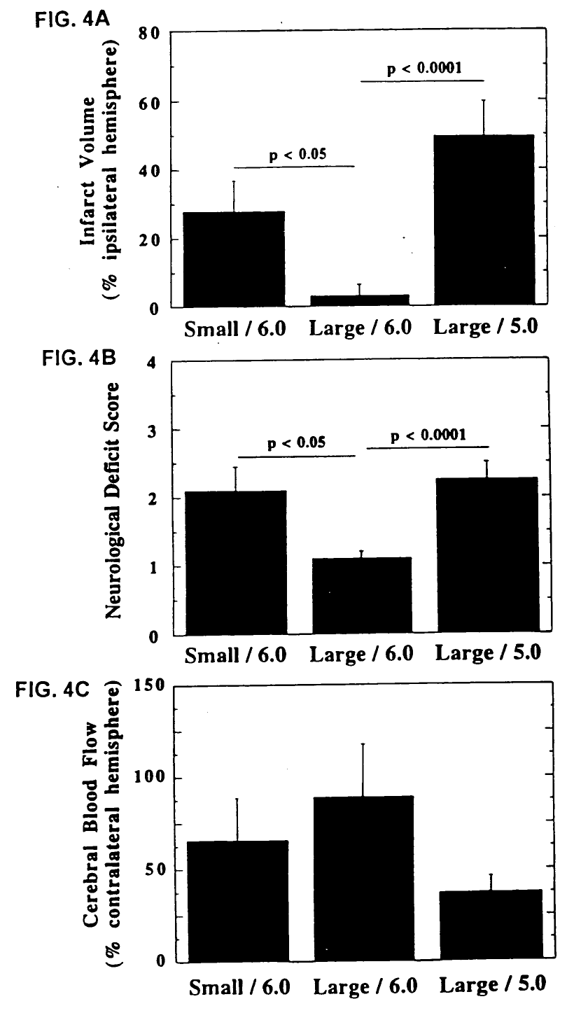

To investigate the effects of mouse size on stroke outcome, mice of two different sizes (23±0.4 g and 31±0.7 g) were subjected to permanent focal cerebral ischemia. To eliminate other potential sources of variability in these experiments, experiments were performed under normothermic conditions in mice of the same strain (CD1), using occluding sutures of identical length and bore (12 mm 6-0 nylon). Under these conditions, small mice (23±0.4 g) sustained consistently large infarct volumes (28±9% of ipsilateral hemisphere). Under identical experimental conditions, large mice (31±0.7 g) demonstrated much smaller infarcts (3.2±3%, p=0.02, FIG. 4A), less morbidity on neurological exam (FIG. 4B), and a tendency to maintain higher ipsilateral cerebral blood flow following infarction than smaller animals (FIG. 4C).

Because it was hypothesized that the reduction in infarct size infarcts in these large animals was related to a mismatch in diameter/length between occluding suture and the cerebral blood vessels, longer/thicker occluding sutures were fashioned (13 mm, 5-0 nylon) for use in these larger mice. Large CD1 mice (34±0.8 g) which underwent permanent occlusion with these larger occluding sutures sustained a marked increase in infarct volumes (50±10% of ipsilateral hemisphere, p<0.0001 compared with large mice infarcted with the smaller occluding suture, FIG. 4A). These larger mice infarcted with larger occluding sutures demonstrated higher neurologic deficit scores (FIG. 4B) and lower ipsilateral cerebral blood flows (FIG. 4C) compared with similarly large mice infarcted with smaller occluding sutures.

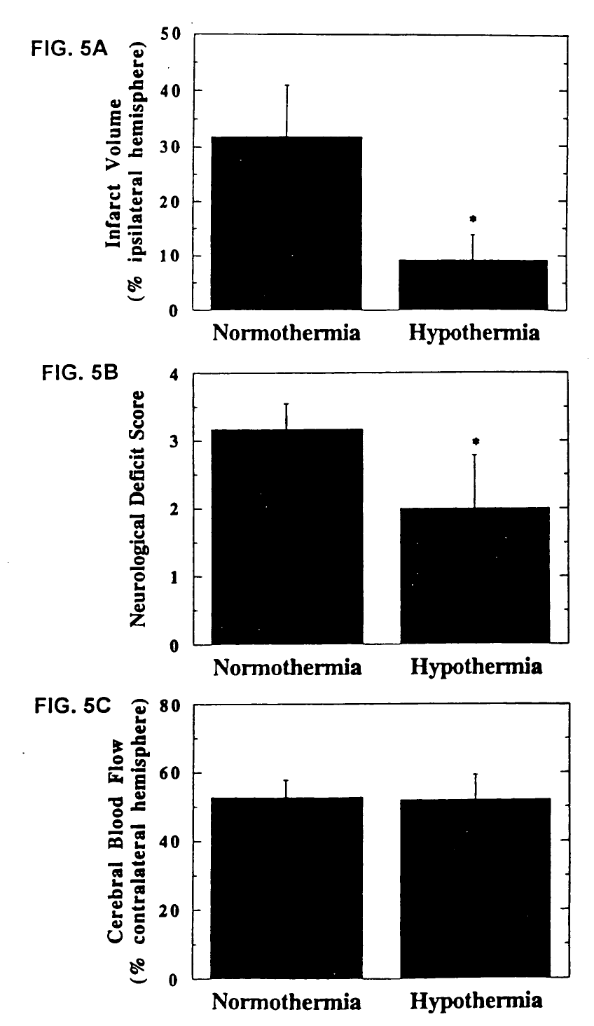

Effects of Temperature: To establish the role of perioperative hypothermia on the stroke volumes and neurologic outcomes following MCA occlusion, small C57/B16 mice (22±0.49) were subjected to permanent MCA occlusion with 12 mm 6-0 gauge suture, with normothermia maintained for two different durations; Group 1 (“Normothermia”) was operated as described above, maintaining temperature at 37° C. from the preoperative period until 90 minutes post-occlusion. Group 2 animals (“Hypothermia”) were maintained at 37° C. from preop to only 10 minutes post-occlusion, as has been described previously (14). Within 45 minutes following removal from the thermocouple-controlled warming incubator, core temperature in this second group of animals dropped to 33.1±0.4° C. (and dropped further to 31.3±0.2° C. at 90 minutes). Animals operated under conditions of prolonged normothermia (Group 1) exhibited larger infarct volumes (32±9%) than hypothermic (Group 2) animals (9.2±5%, p 0.03, FIG. 5A). Differences in infarct volume were mirrored by differences in neurological deficit (3.2±0.4 vs. 2.0±0.8, p=0.02, FIG. 5B), but were largely independent of cerebral blood flow (52±5 vs. 52±7, p NS, FIG. 5C).

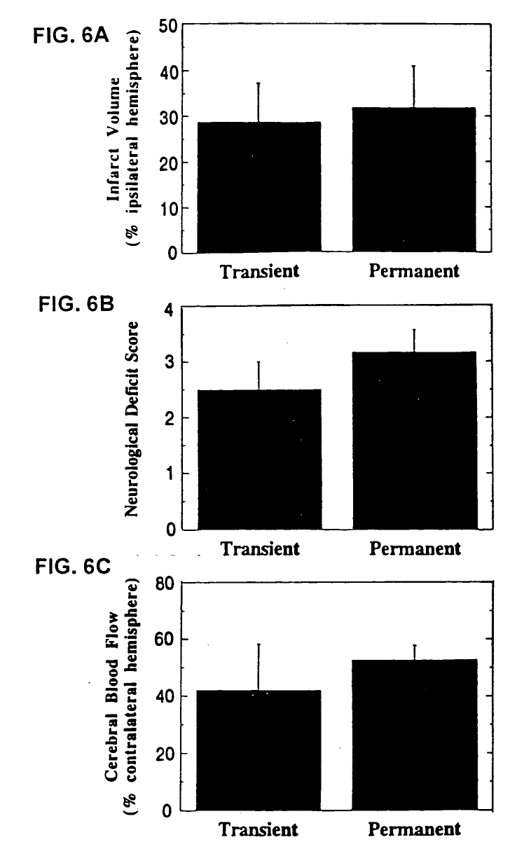

Effects of Transient MCA Occlusion: Because reperfusion injury has been implicated as an important cause of neuronal damage following cerebrovascular occlusion (25), a subset of animals was subjected to a transient (45 minute) period of ischemia followed by reperfusion as described above, and comparisons made with those animals which underwent permanent MCA occlusion. The time of occlusion was chosen on the basis of preliminary studies (not shown) which demonstrated unacceptibly high mortality rates (>85%) with 180 minutes of ischemia and rare infarction (<15%) with 15 minutes of ischemia. To minimize the confounding influence of other variables, other experimental conditions were kept constant (small (22.5±0.3 g) C57/B16 mice were used, the occluding suture consisted of 12 mm 6-0 nyon, and experiments were performed under normothermic conditions). The initial decline in CBF immediately post-occlusion were similar in both groups (16±2% vs 17±3%, for transient vs permanent occlusion groups, respectively, p=NS). Reperfusion was confirmed both by laser doppler (2.3-fold increase in blood flow following removal of the occluding suture to 66±13%), and visually by intracardiac methylene blue dye injection in representative animals. Infarct sizes (29±10% vs. 32±9%), neurologic deficit scores (2.5±0.5 vs. 3.2±0.4), and sacrifice cerebral blood flow (46±18% vs. 53±5%) were quite similar between between animals subjected to transient cerebral ischemia and reperfusion and those subjected to permanent focal cerebral ischemia (p=NS, for all groups) (FIGS. 6A-6C).

Discussion:

The growing availability of genetically altered mice has led to an increasing use of murine models of focal cerebral ischemia to impute specific gene products in the pathogenesis of stroke. Although recent publications describe the use of an intraluminal suture to occlude the middle cerebral artery to create permanent and/or transient cerebral ischemia in mice, there has been only scant description of the necessary modifications of the original technical report in rats (8,14,17-19,24,26). The experiments described herein not only provide a detailed technical explanation of a murine model suitable for either permanent or transient focal middle cerebral artery ischemia, but also address potential sources of variability in the model.

Importance of Strain:

One of the most important potential sources of variability in the murine cerebral ischemia model described herein is related to the strain of animal used. The data suggest that, of the three strains tested, 129J mice are particularly resistant to neurologic injury following MCA occlusion. Although Barone similarly found differences in stroke volumes between 3 strains of mice (BDF, CFW and BALB/C), these differences were ascribed to variations in the posterior communicating arteries in these strains (4). As anatomical differences in cerebrovascular anatomy were not grossly apparent in the study (FIG. 2), the data suggests that non-anatomic strain-related differences are also important in outcome following MCA occlusion.

As stroke outcome differs significantly between 2 strains of mice (129J and C57/B16) commonly used to produce transgenic mice via homologous recombination in embryonic stem cells (11), the data suggest an important caveat to experiments performed with transgenic mice. Because early founder progeny from the creation of transgenic animals with these strains have a mixed 1293/C57/B16 background, ideally experiments should be performed either with sibling controls or after a sufficient number of backcrossings to ensure strain purity.

Importance of Size:

Larger animals require a longer and thicker intralumenal suture to sustain infarction volumes which are consistent with those obtained in smaller animals with smaller occluding sutures. Size matching of animal and suture appear to be important not only to produce consistent cerebral infarction, but whereas too small a suture leads to insufficient ischemia, too large a suture leads to frequent intracerebral hemorrhage and vascular trauma.

The use of animals of similar size is important not only to minimize potential age-related variability in neuronal susceptability to ischemic insult, but also to ensure that small differences in animal size do not obfuscate meaningful data comparison. In this example, it is demonstrated that size differences of as little as 9 grams can have a major impact on infarct volume and neurologic outcome following cerebral ischemia. Further experiments using larger bore occluding suture in larger animals suggest that the increased propensity of smaller animals to have larger strokes was not due to a relative resistance of larger animals to ischemic neuronal damage, but was rather due to small size of the suture used to occlude the MCA in large animals. Although these data were obtained using CD1 mice, similar studies have been performed and found these results to be true with other mouse strains as well, such as C57/B16. Previously published reports use mice of many different sizes (from 21 g to 35 g), as well as different suture diameters and lengths which are often unreported (14,17). The studies indicate that animal and suture size are important methodological issues which must be addressed in scientific reports.

Importance of Temperature:

It has long been recognized that hypothermia protects a number of organs from ischemic injury, including the brain. Studies performed in rats have demonstrated that intraischemic hypothermia up to 1 hour post-MCA occlusion is protective (2,15), reducing both mortality and infarct volumes with temperatures of 34.5 degrees. Although these results have been extrapolated to murine models of cerebral ischemia in that studies often describe maintenance of normothermia in animals, the post-MCA occlusion temperature monitoring periods have been extremely brief (“immediately after surgery” or “10 minutes after surgery”) (4,14). The results indicate that animals fail to autoregulate their temperature beyond these brief durations, becoming severely hypothermic during the postoperative period, and that temperature differences up to 90 minutes following MCA occlusion can have a profound effect on indices of stroke outcome following MCA occlusion (longer durations of normothermia were not studied). While others have ensured normothermia using a feedback system based on rectal temperature similar to the one described herein, the duration of normothermia is often not specified (17). The results argue for clear identification of methods for monitoring and maintaining temperature, as well as the durations involved, so that experimental results can be compared both within and between Centers studying the pathophysiology of stroke.

Transient vs Permanent Occlusion:

The pathophysiology of certain aspects of permanent cerebral ischemia may well be different from that of cerebral ischemia followed by reperfusion, so it was important that a model be described which permitted analysis of either condition. Although differences between these two models were not extensively tested in the current series of experiments, under the conditions tested (45 minutes of ischemia followed by 23 hours of reperfusion), no significant differences were found in any index of stroke, outcome. Variable durations of ischemia and reperfusion have been reported in other murine models of transient cerebral ischemia, with ischemic times ranging from 10 minutes to 3 hours and reperfusion times ranging from 3 to 24 hours (17,24). Studies in rats have shown that short periods of ischemia followed by reperfusion are associated with smaller infarcts than permanent occlusion (21,25). However as the duration of ischemia increases beyond a critical threshold (between 120 and 180 minutes), reperfusion is associated with larger infarcts (7,21,26). For the current series of experiments, the durations of ischemia and reperfusion were chosen so as to obtain infarcts comparable to those observed following permanent MCA occlusion, which is likely to explain why the data failed to show differences between permanent and transient ischemia. These durations in the transient model were chosen after pilot experiments revealed that shorter ischemic durations (15 minutes) rarely led to infarction, whereas 180 minutes of occlusion followed by reperfusion led to massive infarction and nearly 100% mortality within 4-6 hours in normothermic animals (unpublished observation). Although indices of stroke outcome may be measured earlier than 24 hours, the 24 hour observation time was elected because observation at this time permits the study of delayed penumbral death, which is likely to be clinically relevant to the pathophysiology of stroke in humans. Furthermore, 24 hours has been shown in a rat model to be sufficient for full infarct maturation (3,12,15,16).

Technical Aspects of the Murine Model:

Technical aspects of the surgery needed to create focal cerebral ischemia in mice differ in certain important respects from that in rats. Self-retaining retractors, which have been advocated in previous reports in rats (26), are unweildy in mice. Suture-based retraction secured with tape provides a superior alternative. In rats, clip occlusion of the proximal and distal carotid artery after mobilization of the external carotid artery has been reported (26), but creates more carotid trauma and hemmorhage in mice. Without distal internal carotid control, which has not been previously described in mice, backbleeding from the external carotid artery is consistently uncontrollable. Using the techniques described in this paper, surgery can be completed with virtually no blood loss, which is especially important given the small blood volume in mice.

Unlike the rat model, the occlusion and transection of the external carotid artery branches and the pterygopalatine artery in the murine model is achieved with electrocautery alone. Previous reports of murine surgery have been unclear as to whether or not the pterygopalatine artery was taken (17,24). Others have described a method with permanent occlusion of the common carotid artery and trans-carotid insertion of the suture without attention to either the external carotid system or the pterygopalatine artery. While effective for permanent occlusion, this latter method makes reperfusion studies impossible.

The method of reperfusion originally described in the rat requires blind catheter withdrawal without anesthesia (26). When attempted in pilot studies in mice, several animals hemorrhaged. Therefore, a method of suture removal under direct visualization in the anesthetized animal was developed, which not only allows visual confirmation of extracranial carotid artery reperfusion, but also affords meticulous hemostasis. Further, the method permits immediate pre- and post-reperfusion laser doppler flowmetry readings in the anesthetized animal.

These laser doppler flowmetry readings are similar to those described by Kamii et al. and Yang et al. in that the readings are made intermittantly and with the use of a stereotactic micromanipulator (17,24). The readings differ, however, in that the coordinates used (2 mm posterior and 3 and 6 mm lateral to the bregma) are slightly more lateral and posterior than the previously published core and penumbral coordinates (1 mm posterior and 2 mm and 4.5 mm lateral to the bregma). These coordinates, which were adopted based on pilot studies, are the same as those used by Huang et al (14).

### CONCLUSION

These studies demonstrate specific technical aspects of a murine model of focal cerebral ischemia and reperfusion which permits reproducibility of measurements between different laboratories. In addition, these studies provide a framework for understanding important procedural variables which can greatly impact on stroke outcome, which should lead to a clear understanding of non-procedure related differences under investigation. Most importantly, this study points to the need for careful control of mouse strain, animal and suture size, and temperature in experimental as well as control animals. Conditions can be established so that stroke outcome is similar between models of permanent focal cerebral ischemia and transient focal cerebral ischemia, which should facilitate direct comparison and permit the study of reperfusion injury. The model described in this study should provide a cohesive framework for evaluating the results of future studies in transgenic animals, to facilitate an understanding of the contribution of specific gene products in the pathophysiology of stroke.

Table I. Pre- and post-operative physiologic parameters. MAP, mean arterial pressure; pCO₂, partial pressure of arterial CO₂ (mm Hg); ₂ O Sa₂, O saturation (%); Hb, hemoglobin concentration (₉/dl); Preoperative, anesthetized animals prior to carotid dissection; Sham, anesthetized animals undergoing the surgical described in the text, immediately prior to introduction of the occluding suture; Stroke, anesthetized animals undergoing the surgical described in the text, immediately after introduction of the occluding suture. p=NS for all between-group comparisons. (data shown is for small 22 gram C57/B16 mice).

| PARAMETER | PREOPERATIVE | SHAM | STROKE |
|---|---|---|---|
| MAP | 102 ± 5.5 | 94 ± 1.9 | 88 ± 4.9 |
| pH | 7.27 ± 0.02 | 7.23 ± 0.04 | 7.28 ± 0.01 |
| pCO₂ | 46 ± 1.3 | 44 ± 1.3 | 47 ± 3.5 |
| O₂ Sat | 89 ± 1.6 | 91 ± 1.8 | 85 ± 2.2 |
| Hb | 14.6 ± 0.42 | 14.3 ± .12 | 14.2 ± 0.12 |

### REFERENCES

1. Backhaub C, et al. (1992) J Pharmacol Methods 27:27-32.
2. Baker C J, et al. (1992) J Neurosurg 77:438-444.
3. Baker C J, et al. (1995) Neurosurgery 36:1-9.
4. Barone F C, et al. (1993) J Cereb Blood Flow Metab 13:683-692.
5. Bederson J B, et al. (1986) Stroke 17:1304-1308.
6. Bederson J B, et al. (1986) Stroke 17:472-476.
7. Buchan A M, et al. (1992) Stroke 23:273-279.
8. Chan P H, et al. (1993) NeuroReport 5:293-296.
9. Chiamulera C, et al. (1993) Brain Res 606:251-258.
10. Dirnagl U, et al. (1989) J Cereb Blood Flow Metab 9:589-596.
11. Donehower L A, et al. (1992) Nature 356:215-221.
12. Frazzini V I, et al. (1994) Neurosurgery 34:1040-1046.
13. Ginsberg M D and Busto R (1989) Stroke 20:1627-1642.
14. Huang Z, et al. (1994) Science 265:1883-1885.
15. Kader A, et al. (1992) Neurosurgery 31:1056-1061.
16. Kader A, et al. (1993) Stroke 24:1709-1716.
17. Kamii H, et al. (1994) J Cereb Blood Flow Metab 14:478-486.
18. Kinouchi H, et al. (1991) Proc Natl Acad Sci 88: 11158-11162.
19. Martinou J-C, et al. (1994) Neuron 13:1017-1030.
20. Memezawa H, et al. (1992) Stroke 23:552-559.
21. Menzies S A, et al. (1992) Neurosurgery 31:100-107.
22. Tamura A, et al. (1981) J Cereb Blood Flow Metabol 1: 53-60.
23. Welsh F A, et al. (1987) J Neurochem 49:846-851.
24. Yang G, et al. (1994) Stroke 25:165-170.
25. Yang G-Y and Betz A L (1994) Stroke 25: 1658-65.
26. Zea-Longa E, et al. (1989) Stroke 20:84-91.

### EXAMPLE 2

### Factor IXai

Factor IX is a clotting factor which exists in humans and other mammals, and is an important part of the coagulation pathway. In the normal scheme of coagulation, Factor IX is activated by either Factor XIa or a tissue factor/VIIa complex to its active form, Factor IXa. Factor IXa then can activate Factor X, which triggers the final part of the coagulation cascade, leading to thrombosis. Because Factor X can be activated by one of two pathways, either the extrinsic (via VIIa/tissue factor) or the intrinsic pathways (via Factor IXa), we hypothesized that inhibiting Factor IXa might lead to impairment of some forms of hemostasis, but leave hemostasis in response to tissue injury intact. In other words, it might lead to blockade of some types of clotting, but might not lead to excessive or unwanted hemorrhage. Factor IXai is Factor IXa which has been chemically modified so as to still resemble Factor IXa (and therefore, can compete with native Factor IXa), but which lacks its activity. This can “overwhelm” or cause a competitive inhibition of the normal Factor IXa-dependent pathway of coagulation. Because Factor IXa binds to endothelium and platelets and perhaps other sites, blocking the activity of Factor IXa may also be possible by administering agents which interfere with the binding of Factor IXa (or by interfering with the activation of Factor IX).

In stroke and other ischemic disorders, there may be clinical benefit derived by lysing an existing thrombus, but there is also the potentially devastating complication of hemorrhage. In the current experiments, the mouse model of cerebral ischemia and reperfusion (stroke) was used. Mice received an intravenous bolus of 300 μg/kg of Factor IXai just prior to surgery. Strokes were created by intraluminal occlusion of the right middle cerebral artery. When stroke outcomes were measured 24 hours later, animals that had received Factor IXai had smaller infarct volumes, improved cerebral perfusion, less neurological deficits, and reduced mortality compared with controls which underwent the same surgery but which did not receive Factor IXai. (See Table II.) It was also noted that the Factor IXai animals were free of apparent intracerebral hemorrhage. By contrast, intracerebral hemorrhage was occasionally noted in the control animals not receiving Factor IXai.

TABLE II

| | |
|---|---|
| | Experimental |
| Control | (Factor IXai) |

| | mean | sd | mean | sd | stats |
|---|---|---|---|---|---|
| weight | 26.91 | 3.21 | 25.25 | 2.49 | 0.14 |
| dopp | 0.96 | 0.24 | 1.04 | 0.35 | 0.52 |
| occ dop 1 | 0.18 | 0.07 | 0.16 | 0.08 | 0.60 |
| occ dop 2 | 0.40 | 0.22 | 0.43 | 0.20 | 0.68 |
| reper dop | 0.55 | 0.42 | 0.53 | 0.30 | 0.89 |
| sac dop | 0.38 | 0.25 | 0.75 | 0.31 | 0.02 |
| grade | 2.22 | 0.67 | 1.67 | 0.49 | |
| I/C Ratio | 1.18 | 0.20 | 1.08 | | |
| inf vol | 21.16 | 25.14 | 3.47 | 12.03 | 0.0452 |
| count | 11 | | 16 | | |

Abbreviations:
dopp = doppler;
occ dop = occlusion doppler;
reper dop = reperfusion doppler;
sac dop = sacrifice doppler.

### EXAMPLE 3

### Active-Site Blocked Factor IXa Limits Microvascular Thrombosis and Cerebral Injury in Murine Stroke without Increasing Intracerebral Hemorrhage

The clinical dilemma in stroke treatment is that agents which restore vascular patency increase the risk of intracerebral hemorrhage. Active-site blocked Factor IXa (IXai) competes with native Factor IXa to inhibit assembly of Factor IXa into the intrinsic Factor X activation complex. When pretreated with Factor IXai, mice subjected to focal cerebral ischemia and reperfusion demonstrated reduced microvascular fibrin and platelet deposition, increased cerebral perfusion, and significantly smaller cerebral infarcts than vehicle-treated controls. Factor IXai-mediated cerebroprotection was dose-dependent, not associated with intracerebral hemorrhage at therapeutically effective doses, and was seen even when Factor IXai was administered after the onset of cerebral ischemia. Administration of Factor IXai represents a new strategy to treat stroke in evolution without increasing the risk of intracerebral hemorrhage. intracerebral hemorrhage.

Introduction

Timely reestablishment of blood flow to ischemic brain represents the current treatment paradigm for acute stroke ¹⁻³. Administration of a thrombolytic agent, even when given under optimal conditions, may not achieve this desired clinical result. Perfusion often fails to return to preischemic levels (postischemic hypoperfusion), suggesting that ischemic injury is not produced solely by the original occlusion, but that there is also an element of microcirculatory failure. In addition, thrombolysis of acute stroke is associated with an increased risk of intracerebral hemorrhage (ICH) ¹⁻⁴, indicating that there remains a clear need to identify new agents which can promote reperfusion without increasing the risk of ICH.

Following an ischemic event, the vascular wall is modified from its quiescent, anti-adhesive, antithrombotic state, to one which promotes leukocyte adhesion and thrombosis. In acute stroke, active recruitment of leukocytes by adhesion receptors expressed in the ipsilateral microvasculature, such as ICAM-1 ⁵ and P-selectin ⁶, potentiates postischemic hypoperfusion. However, experiments with mice-deletionally mutant for each of these genes demonstrate that even in their absence, postischemic cerebral blood flow (CPF) returns only partially to baseline, suggesting the existence of additional mechanisms responsible for postischemic cerebrovascular no-reflow. To explore this possibility, the first set of experiments was designed to test the hypothesis that local thrombosis occurs at the level of the microvasculature (distal to the site of primary occlusion) in stroke.

To assess the deleterious consequences of microvascular thrombosis in stroke, the second set of experiments tested the hypothesis that selective blockade of the intrinsic pathway of coagulation could limit microvascular thrombosis, thereby protecting the brain in stroke. The strategy of selective inhibition of the intrinsic pathway of coagulation was chosen because it is primarily responsible for intravascular thrombosis. Heparin, hirudin, and fibrinolytic agents interfere with the final common pathway of coagulation to inhibit the formation or accelerate the lysis of fibrin, and therefore increase the propensity for ICH. We hypothesized that selective blockade of IXa/VIIIa/X activation complex assembly might provide a novel mechanism to limit intravascular thrombosis while preserving mechanisms of extravascular hemostasis by the extrinsic/tissue factor pathway of coagulation which may be critical in infarcted brain tissue or adjacent regions where small vessels are friable and subject to rupture. We used a novel strategy in which a competitive inhibitor of Factor IXa (active-site blocked IXa, or IXai) was given to mice subjected to stroke to test the hypothesis that it would improve stroke outcome without increasing ICH.

Methods

Murine stroke model: Transient focal cerebral ischemia was induced in mice by intralumenal occlusion of the middle cerebral artery (45 minutes) and reperfusion (22 hrs) as previously reported ⁷. Serial measurements of relative cerebral blood flow (CBF) were recorded via laser doppler flowmetry 7, and infarct volumes (t ipsilateral hemisphere) determined by planimetric/volumetric analysis of triphenyl tetrazolium chloride (TTC)-stained serial cerebral sections ⁷.

¹¹¹Indium-platelet studies: Platelet accumulation was determined using ¹¹¹Indium labeled platelets, collected and prepared as previously described ⁸. Immediately prior to surgery, mice were given 5×10^{6 111}In-labeled-platelets intravenously; deposition was quantified after 24 hours by as ipsilateral cpm/contralateral cpm.

Fibrin immunoblotting/immunostaining: The accumulation of fibrin was measured following sacrifice (of fully heparinized animals) using immunoblotting/immunostaining procedures which have been recently described and validated ⁹. Because fibrin is extremely insoluble, brain tissue extracts were prepared by plasmin digestion, then applied to a standard SDS-polyacrylamide gel for electrophoresis, followed by immunoblotting using a polyclonal rabbit anti-human antibody prepared to gamma-gamma chain dimers present in cross-linked fibrin which can detect murine fibrin, with relatively little cross-reactivity with fibrinogen ¹⁰. Fibrin accumulation was reported as an ipsilateral to contralateral ratio. In additional experiments, brains were embedded in paraffin, sectioned, and immunostained using the same anti-fibrin antibody.

Spectrophotometric hemoglobin assay and visual ICH score: ICH was quantified by a spectrophotometric-based assay which we have developed and validated 1,12 In brief, mouse brains were homogenized, sonicated, centrifuged, and methemoglobin in the supernatants converted (using Drabkin's reagent) to cyanomethemoglobin, the concentration of which was assessed by measuring O.D. at 550 nm against a standard curve generated with known amounts of hemoglobin. Visual scoring of ICH was performed on 1 mm serial coronal sections by a blinded observer based on maximal hemorrhage diameter seen on any of the sections [ICH score 0, no hemorrhage; 1, <1 mm; 2, 1-2 mm; 3, >2-3 mm; 4, >3 mm].

Preparation of Factor IXai ¹³: Factor IXai was prepared by selectively modifying the active site histidine residue on Factor IXa, using dansyl-glu-gly-arg-chloromethylketone. Proplex was applied to a preparative column containing immobilized calcium-dependent monoclonal antibody to Factor IX. The column was washed, eluted with EDTA-containing buffer, and Factor IX in the eluate (confirmed as a single band on SDS-PAGE) was then activated by applying Factor XIa (incubating in the presence of CaCl₂). Purified Factor IXa was reacted with a 100-fold molar excess of dansyl-glu-gly-arg chloromethylketone, and the mixture dialyzed. The final product (IXai), devoid of procoagulant activity, migrates identically to IXa on SDS-PAGE. This material (Factor IXai) was then used for experiments following filtration (0.2 μm) and chromatography on DeToxi-gel columns, to remove any trace endotoxin contamination (in sample aliquots, there was no detectable lipopolysaccharide). IXai was subsequently frozen into aliquots at −80° C. until the time of use. For those experiments in which IXai was used, it was given as a single intravenous bolus at the indicated times and at the indicated doses.

Results

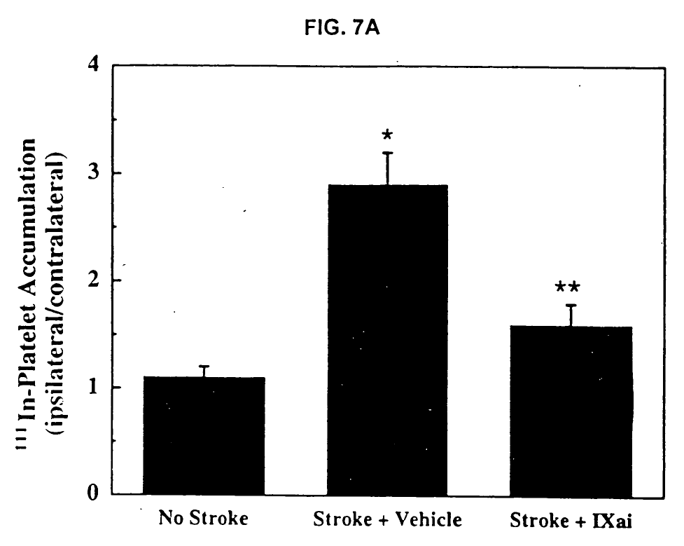

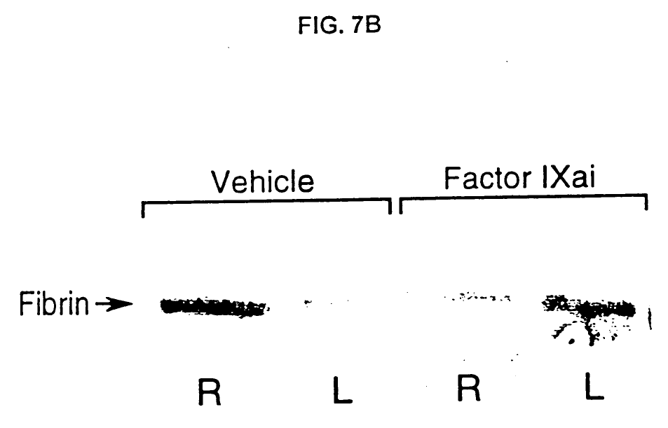

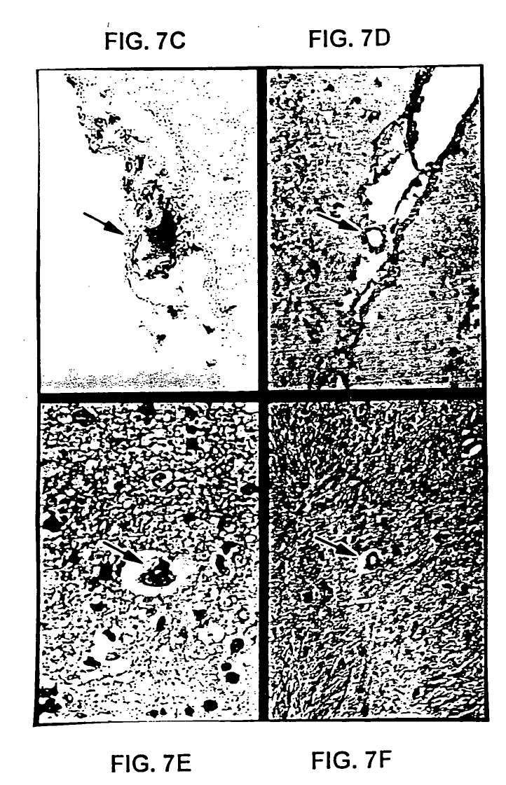

To create a stroke in a murine model, a suture is introduced into the cerebral vasculature so that it occludes the orifice of the right middle cerebral artery, rendering the subtended territory ischemic. By withdrawing the suture after a 45 minute period of occlusion, a reperfused model of stroke is created; mice so treated demonstrate focal neurological deficits as well as clear-cut areas of cerebral infarction. Because the occluding suture does not advance beyond the major vascular tributary (the middle cerebral artery), this model provides an excellent opportunity to investigate “downstream” events that occur within the cerebral microvasculature in response to the period of interrupted blood flow. Using this model, the role of microvascular thrombosis was investigated as follows. To demonstrate that platelet-rich thrombotic foci occur within the ischemic cerebral hemisphere, ¹¹¹In-labeled platelets were administered to mice immediately prior to the introduction of the intraluminal occluding suture, to track their deposition during the ensuing period of cerebral ischemia and reperfusion. In animals not subjected to the surgical procedure to create stroke, the presence of platelets was approximately equal between the right and left hemispheres, as would be expected [FIG. 7A, left bar]. However, when animals were subjected to stroke (and received only vehicle to control for subsequent experiments), radiolabeled platelets preferentially accumulated in the ischemic (ipsilateral) hemisphere, compared with significantly less deposition in the contralateral (nonischemic) hemisphere [FIG. 7A, middle bar]. These data support the occurrence of platelet-rich thrombi in the ischemic territory. When Factor IXai is administered to animals prior to introduction of the intraluminal occluding suture, there is a significant reduction in the accumulation of radiolabelled platelets in the ipsilateral hemisphere [FIG. 7A, right bar].

Another line of evidence also supports the occurrence of microvascular thrombosis in stroke. This data comes from the immunodetection of fibrin, using an antibody directed against a neoepitope on the gamma-gamma chain dimer of cross-linked fibrin. Immunoblots demonstrate a band of increased intensity in the ipsilateral (right) hemisphere of vehicle-treated animals subjected to focal cerebral ischemia and reperfusion [FIG. 7B, “Vehicle”]. In animals treated with Factor IXai (300 μg/kg) prior to stroke, there is no apparent increase in the ipsilateral accumulation of fibrin [FIG. 7B, “Factor IXai”]. To demonstrate that fibrin accumulation was due to the deposition of intravascular fibrin (rather than due to nonspecific permeability changes and exposure to subendothelial matrix), fibrin immunostaining clearly localized the increased fibrin to the lumina of ipsilateral intracerebral microvessels [FIG. 7C].

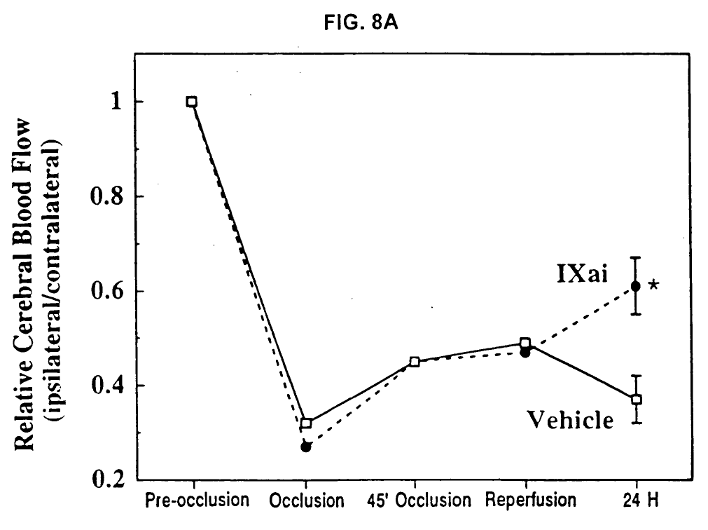

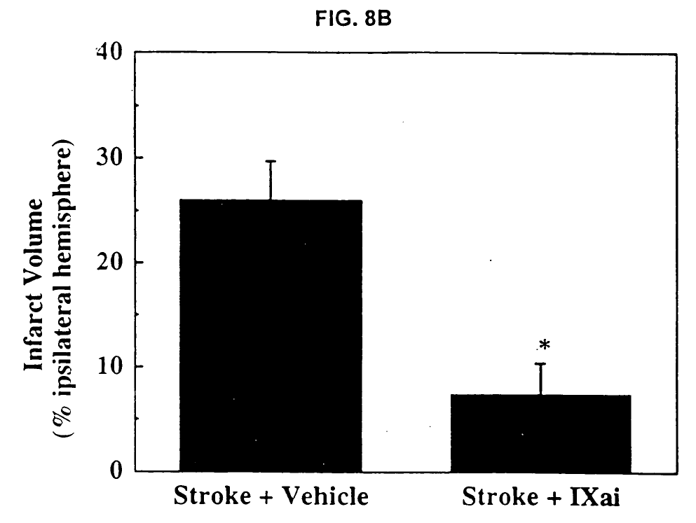

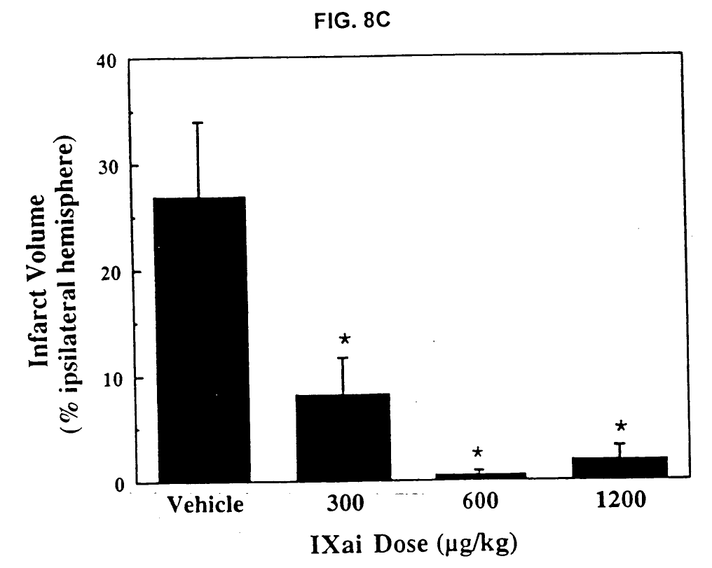

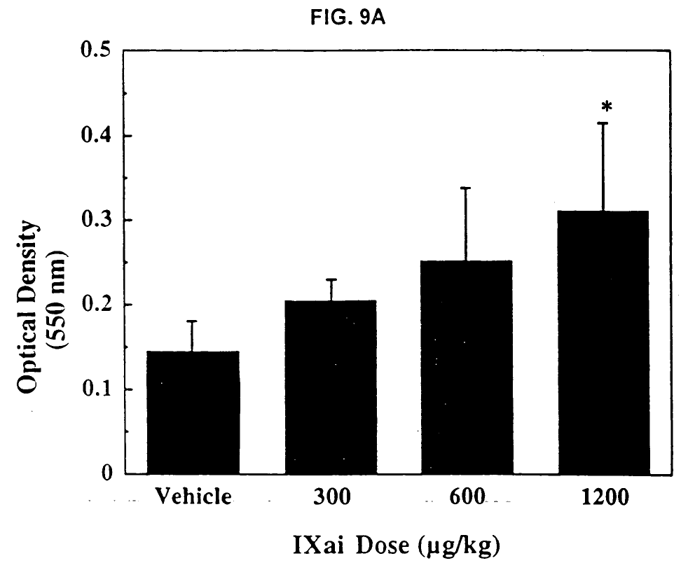

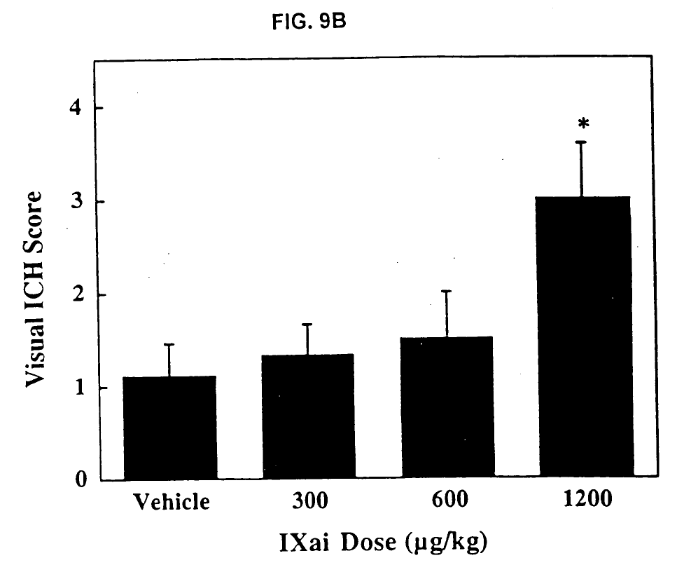

To investigate whether Factor IXai can limit intracerebral thrombosis and restore perfusion, IXai was given to mice immediately prior to stroke (300 μg/kg). These experiments demonstrate both a reduction in ¹¹¹In-platelet accumulation in the ipsilateral hemisphere [FIG. 8A] as well as decreased evidence of intravascular fibrin by immunostaining. Furthermore, there is a significant increase in CBF by 24 hours, suggesting the restoration of microvascular patency by Factor IXai [FIG. 8A]. The clinical relevance of this observation is underscored by the ability of Factor IXai to reduce cerebral infarct volumes [FIG. 8B]. These beneficial effects of Factor IXai were dose Dependent, with 600 μg/kg being the optimal dose [FIG. 8C]. Because the development of ICH is a major concern with any anticoagulant strategy in the setting of stroke, the effect of IXai on ICH was measured using our recently validated spectrophotometric method for quantifying ICH ^{11,12}. These data indicate that at the lowest doses (and the most effective ones), there is no significant increase in ICH [FIG. 9A]. At the highest dose tested (1200 μg/kg), there is an increase in ICH, which was corroborated by a semiquantitative visual scoring method which we have also recently reported [FIG. 9B] ^{11,12}.

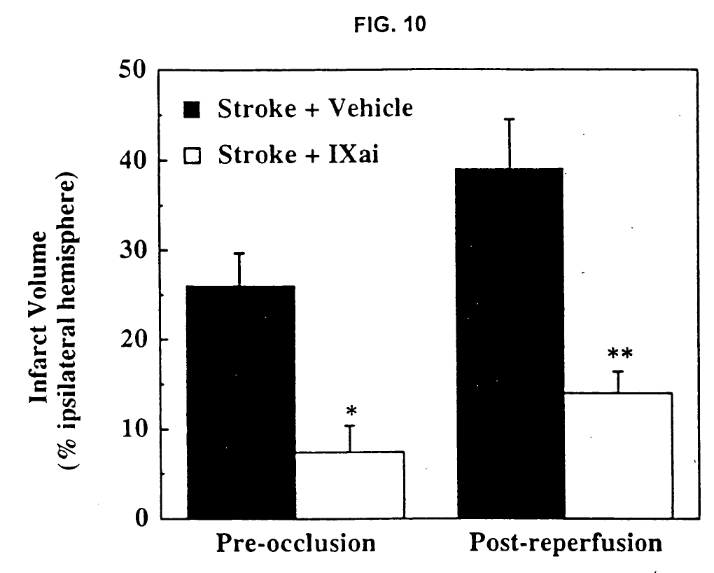

Because therapies directed at improving outcome from acute stroke must be given after clinical presentation, and because fibrin continues to form following the initial ischemic event in stroke, we tested whether IXai might be effective when given following initiation of cerebral ischemia. IXai given after middle cerebral artery occlusion (following removal of the occluding suture) provided significant cerebral protection judged by its ability to significantly reduce cerebral infarction volumes compared with vehicle-treated controls [FIG. 10].

Discussion

The data in these studies demonstrate clear evidence of intravascular thrombus formation (both platelets and fibrin) within the post-ischemic cerebral microvasculature. The pathophysiological relevance of microvascular thrombosis in stroke is underscored by the ability of Factor IXai to reduce microvascular thrombosis (both platelet and fibrin accumulation are reduced, with an attendant increase in postischemic CBF) and to improve stroke outcome. These potent antithrombotic actions of Factor IXai are likely to be clinically significant in the setting of stroke, because Factor IXai not only reduces infarct volumes in a dose-dependent manner, but it does so even when given after the onset of stroke. In addition, at clinically relevant doses, treatment with Factor IXai does not cause an increase in ICH, making selective inhibition of Factor IXa/VIIIa/X activation complex assembly with Factor IXai an attractive target for stroke therapy in humans.

There are a number of reasons why targetted anticoagulant strategies might be an attractive alternative to the current use of thrombolytic agents in the management of acute stroke, because of their checkered success in clinical trials. Theoretically, an ideal treatment for acute stroke would prevent the formation or induce dissolution of the fibrin-platelet mesh that causes microvascular thrombosis in the ischemic zone without increasing the risk of intracerebral hemorrhage. However, thrombolytic agents which have been studied in clinical trials of acute stroke have consistently increased the risk of intracerebral hemorrhage 1-4. Streptokinase, given in the first several (<6) hours following stroke onset, was associated with an increased rate of hemorrhagic transformation (up to 67%); although there was increased early mortality, surviving patients suffered less residual disability. Administration of tissue-type plasminogen activator (tPA) within 7 hours (particularly within 3 hours) of stroke onset resulted in increased early mortality and increased rates of hemorrhagic conversion (between 7-20%), although survivors demonstrated less residual disability. In order to develop improved anticoagulant or thrombolytic therapies, several animal models of stroke have been examined. These models generally consist of the administration of clotted blood into the internal carotid artery followed by administration of a thrombolytic agent. In rats, tPA administration within 2 hours of stroke improved cerebral blood flow and reduced infarct size by up to 77% ^{14,15}, In a similar rabbit embolic stroke model, tPA was effective at restoring blood flow and reducing infarct size, with occasional appearance of intracerebral hemorrhage ^{16,17}. However, although there are advantages to immediate clot dissolution, these studies (as well as the clinical trials of thrombolytic agents) indicate that there is an attendant increased risk of intracerebral hemorrhage with this therapeutic approach.

Because of the usually precipitous onset of ischemic stroke, therapy has been targetted primarily towards lysing the major fibrinous/atheroembolic debris which occludes a major vascular tributary to the brain. However, as the current work demonstrates, there is an important component of microvascular thrombosis which occurs downstream from the site of original occlusion, which is likely to be of considerable pathophysiological significance for post-ischemic hypoperfusion (no-reflow) and cerebral injury in evolving stroke. This data is in excellent agreement with that which has been previously reported, in which microthrombi have been topographically localized to the ischemic region in fresh brain infarcts ¹⁸. The use of an agent which inhibits assembly of the Factor IXa/VIIIa/X activation complex represents a novel approach to limiting thrombosis which occurs within microvascular lumena, without impairing extravascular hemostasis, the maintenance of which may be critical for preventing ICH. In the current studies, treatment with Factor IXai reduces microvascular platelet and fibrin accumulation, improves postischemic cerebral blood flow, and reduces cerebral infarct volumes in the setting of stroke without increasing ICH.

The potency of Factor IXai as an anticoagulant agent stems from the integral role of activated Factor IX in the coagulation cascade. Not only does a strategy of Factor IXa blockade appear to be effective in the setting of stroke, but it also appears to be effective at preventing progressive coronary artery occlusion induced following the initial application of electric current to the left circumflex coronary artery in dogs ¹³. As in those studies, in which Factor IXai did not prolong the pro time.

The data which demonstrate that IXai given after the onset of stroke is effective leads to another interesting hypothesis, that the formation of thrombus represents a dynamic equilibrium between the processes of ongoing thrombosis and ongoing fibrinolysis. Even under normal (nonischemic) settings, this dynamic equilibrium has been shown to occur in man ¹⁵. The data in the current studies, which show that Factor IXai is effective even when administered after the onset of stroke, suggests that this strategy restores the dynamic equilibrium, which is shifted after cerebral ischemia to favor thrombosis, back towards a more quiescent (antithrombotic) vascular wall phenotype.

As a final consideration, even if thrombolysis successfully removes the major occluding thrombus, and/or anticoagulant strategies are effective to limit progressive microcirculatory thrombosis, blood flow usually fails to return to pre-ischemic levels. This is exemplified by data in the current study, in which although CBF is considerably improved by Factor IXai (which limits fibrin/platelet accumulation), CBF still does not return to preischemic levels. This data supports the existence of multiple effector mechanisms for postischemic cerebral hypoperfusion, including postischemic neutrophil accumulation and consequent microvascular plugging, with P-selectin and ICAM-1 expression by cerebral microvascular endothelial cells being particularly germane in this regard 5,6. When looked at from the perspective of leukocyte adhesion receptor expression, even when these adhesion receptors are absent, CBF levels are improved following stroke compared with controls but do not return to preischemic levels. Taken together, these data suggests that microvascular thrombosis and leukocyte adhesion together contribute to postischemic cerebral hypoperfusion.

In summary, administration of a competitive inhibitor of Factor IXa, active-site blocked Factor IXa, represents a novel therapy for the treatment of stroke. This therapy not only reduces microcirculatory thrombosis, improves postischemic cerebral blood flow, and reduces cerebral tissue injury following stroke, but it can do so even if given after the onset of cerebral ischemia and without increasing the risk of ICH. This combination of beneficial properties and relatively low downside risk of hemorrhagic transformation makes this an extremely attractive approach for further testing and potential clinical trials in human stroke.

### REFERENCES

1. New Engl. J. Med. (1995)333:1581-1587.
2. Hacke W, et al. (1995) JAMA 274(13):1017-1025.
3. del Zoppo G J (1995) N. Engl. J. Med. 333(13):1632-1633.
4. Hommel M, et al. (1996) N. Engl. J. Med. 335:145-150.
5. Connolly E S Jr, et al. (1996) J. Clin. Invest. 97:209-216.
7. Connolly E S Jr, et al. (1996) Neurosurg 38(3):523-532.
8. Naka Y, et al. (1995) Circ. Res. 76:900-906.
9. Lawson C A, et al. (1997) J. Clin. Invest. 99:1729-1738.
10. Lahiri B, et al. (1981) Thromb. Res. 23:103-112.
12. Choudhri T F, et al. (1997) Annual Meeting Joint Section on Cerebrovascular Surgery.
13. Benedict C R, et al. (1991) J Clin Invest 88:1760-1765.
14. Papadopoulos S M, et al. (1987) J Neurosurg 67:394-398.
15. Overgaard K, et al. (1993) Neurol Res 15:344-349.
16. Carter L P, et al. (1992) Stroke 23:883-888.
17. Phillips D A, et al. (1990) Stroke 21:602-605.
18. Heye N, et al. (1992) Acta Neurologica Scandinavica 86:450-454.
19. Nossel H L (1981) Nature 1981;291:165-167

### EXAMPLE 4

### Active-site Blocked Factor IXa Limits Microvascular Thrombosis and Cerebral Injury in Murine Stroke without Increasing Intracerebral Hemorrhage

[Please note the following abbreviations: CBF, cerebral blood flow; Factor IXai, active-site blocked factor IXa; ICAM-1, intercellular adhesion molecule-1; ICH, intracerebral hemorrhage; tPA, tissue plasminogen activator; TTC, triphenyl tetrazolium chloride.]

The clinical dilemma in stroke treatment is that agents which restore vascular patency increase the risk of intracerebral hemorrhage (ICH). It was hypothesized that inhibiting cerebral microvascular thrombosis by inhibiting intrinsic Factor IX-dependent coagulation may restore vascular patency in stroke without impairing extrinsic hemostatic mechanisms that may limit ICH. Active-site blocked Factor IXa (IXai) was formed from purified factor IXa by dansylation of its active site, to compete with native Factor IXa to inhibit assembly of Factor IXa into the intrinsic Factor X activation complex. Although in vitro, Factor IXai had little effect on the PT or PTT, it prolonged clotting time in an assay in which Factor IX-deficient plasma was reconstituted with Factor IX. When pretreated with Factor IXai, mice subjected to middle cerebral artery occlusion and reperfusion demonstrated an 1.8-fold reduced microvascular fibrin and platelet deposition, 2.4-fold increased cerebral perfusion, and significantly smaller cerebral infarcts 3.5-fold than vehicle-treated controls (p<0.05, 0.05, and 0.05, respectively). Factor IXai-mediated cerebroprotection was not associated with ICH at therapeutically effective doses, and was seen even when Factor IXai was administered after the onset of cerebral ischemia. In contrast, a less targeted anticoagulant strategy with heparin reduced cerebral infarction volumes only at doses which increased ICH. Administration of Factor IXai represents a new strategy to treat stroke in evolution without increasing the risk of ICH. The apparent efficacy of Factor IXai when given after stroke suggests that microvascular thrombosis continues to evolve (and may be inhibited) even after occlusion of a major vascular tributary, thereby broadening the potential therapeutic window for its administration.

Timely reestablishment of blood flow to ischemic brain represents the current treatment paradigm for acute stroke (1-3). Administration of a thrombolytic agent, even when given under optimal conditions, may not achieve this desired clinical result. Perfusion often fails to return to preischemic levels (postischemic hypoperfusion), suggesting that ischemic injury is not produced solely by the original occlusion, but that there is also an element of microcirculatory failure. Small early trials of a general anticoagulant strategy involving heparin in stroke were disappointing in that the use of heparin was either ineffective and/or associated with an unacceptably high incidence of hemorrhagic conversion (in up to 14% of treated patients) (4-7,7-9). Although the current vogue is to use recombinant tissue plasminogen activator (tPA) to achieve thrombolysis in ischemic stroke, this approach is also associated with an increased risk of intracerebral hemorrhage (ICH) (1-3,10). Consequently, there remains a clear need to identify new agents which can promote reperfusion without increasing the risk of ICH.

Following an ischemic event, the vascular wall is modified from its quiescent, anti-adhesive, antithrombotic state, to one which promotes leukocyte adhesion and thrombosis. In acute stroke, active recruitment of leukocytes by adhesion receptors expressed in the ipsilateral microvasculature, such as intercellular adhesion molecule-1 (ICAM-1) (11) and P-selectin (12), potentiates postischemic hypoperfusion. However, experiments with mice deletionally mutant for each of these genes demonstrate that even in their absence, postischemic cerebral blood flow (CBF) returns only partially to baseline after removal of an intraluminal middle cerebral artery occluding suture. This indicates that there exist additional mechanisms responsible for postischemic cerebrovascular no-reflow, especially the possibility that local thrombosis occurs at the level of the microvasculature (distal to the site of primary occlusion) in stroke. Furthermore, if the ischemic insult is particularly severe, reflow continues to worsen over the time subsequent to withdrawal of the occluding suture, suggesting ongoing vascular obstructive processes (such as de novo thrombosis).

These observations provide the basis for exploring the role of general thrombolytic and/or anticoagulant strategies in the murine model of stroke. However, compelling clinical data indicate that agents which selectively limit thrombosis in stroke without increasing ICH will offer unique advantages which are not seen with any agent tested so far. Because the subendothelial vascular matrix in brain tissue is a rich source of tissue factor, we hypothesized that anticoagulant strategis which do not impair tissue-factor mediated hemostatic events might provide a novel means to reduce thrombosis in the microvascular lumen, yet not impair the ability of friable postischemic cerebral microvessels to form effective hemostatic plugs to limit ICH. Heparin or hirudin, which interfere with the final common pathway of coagulation, or thrombolytic agents, which nonselectively lyse fibrin, do not offer the theroretical advantage offered by targeting the intrinsic limb of the coagulation cascade. The current experiments test the hypothesis that selective blockade of IXa/VIIIa/X activation complex assembly using a novel strategy in which a competitive inhibitor of Factor IXa (active-site blocked IXa, Factor IXai), might provide a novel mechanism to limit intravascular thrombosis while preserving mechanisms of extravascular hemostasis, thereby improving stroke outcome without increasing ICH.

Methods

Murine stroke model: Transient focal cerebral ischemia was induced in mice by intralumenal occlusion of the middle cerebral artery (45 minutes) and reperfusion (24 hrs) as previously reported (13). Serial measurements of relative cerebral blood flow (CBF) were recorded via laser doppler flowmetry (13), and infarct volumes (% ipsilateral hemisphere) determined by planimetric/volumetric analysis of triphenyl tetrazolium chloride (TTC)-stained serial cerebral sections (13).

¹¹¹Indium-platelet studies: Platelet accumulation was determined using ¹¹¹Indium labeled platelets, collected and prepared as previously described (14). Immediately prior to surgery, mice were given 5×10^{6 111}In-labeled-platelets intravenously; deposition was quantified after 24 hours by as ipsilateral cpm/contralateral cpm.

Fibrin immunoblotting/immunostaining: The accumulation of fibrin was measured following sacrifice (of fully heparinized animals) using immunoblotting/immunostaining procedures which have been recently described and validated (15). Because fibrin is extremely insoluble, brain tissue extracts were prepared by plasmin digestion, then applied to a standard SDS-polyacrylamide gel for electrophoresis, followed by immunoblotting using a polyclonal rabbit anti-human antibody prepared to gamma-gamma chain dimers present in cross-linked fibrin which can detect murine fibrin, with relatively little cross-reactivity with fibrinogen (16). Fibrin accumulation was reported as an ipsilateral to contralateral ratio. In additional experiments, brains were embedded in paraffin, sectioned, and immunostained using the same anti-fibrin antibody.

Spectrophotometric hemoglobin assay and visual ICH score: ICH was quantified by a spectrophotometric-based assay which we have developed and validated (17). In brief, mouse brains were homogenized, sonicated, centrifuged, and methemoglobin in the supernatants converted (using Drabkin's reagent) to cyanomethemoglobin, the concentration of which was assessed by measuring O.D. at 550 nm against a standard curve generated with known amounts of hemoglobin.

Preparation of Factor IXai (18): Factor IXai was prepared by selectively modifying the active site histidine residue on Factor IXa, using dansyl-glu-gly-arg-chloromethylketone. Proplex was applied to a preparative column containing immobilized calcium-dependent monoclonal antibody to Factor IX. The column was washed, eluted with EDTA-containing buffer, and Factor IX in the eluate (confirmed as a single band on SDS-PAGE) was then activated by applying Factor XIa (incubating in the presence of CaCl₂). Purified Factor Ixa was reacted with a 100-fold molar excess of dansyl-glu-gly-arg chloromethylketone, and the mixture dialyzed. The final product (IXai), devoid of procoagulant activity, migrates identically to IXa on SDS-PAGE. This material (Factor IXai) was then used for experiments following filtration (0.2 μm) and chromatography on DeToxi-gel columns, to remove any trace endotoxin contamination (in sample aliquots, there was no detectable lipopolysaccharide). Factor IXai was subsequently frozen into aliquots at −80° C. until the time of use. For those experiments in which Factor IXai was used, it was given as a single intravenous bolus at the indicated times and at the indicated doses.

Modified Cephalin Clotting Time Equal volumes of factor IX-deficient plasma (American Diagnostica Inc.) and 0.024M celite in 0.05M barbital buffer (Sigma) were combined in silicone-coated glass tubes (Sigma) for 2 minutes at 37° C. To this mixture, an equal volume of 1:16 (v/v) cephalin (10 mg/ml, Sigma) in 0.05M barbital buffer was added, followed by a one-half volume of sample plasma. After the addition of calcium chloride to a final concentration of 0.001M, the time required for clot formation was determined.

Results

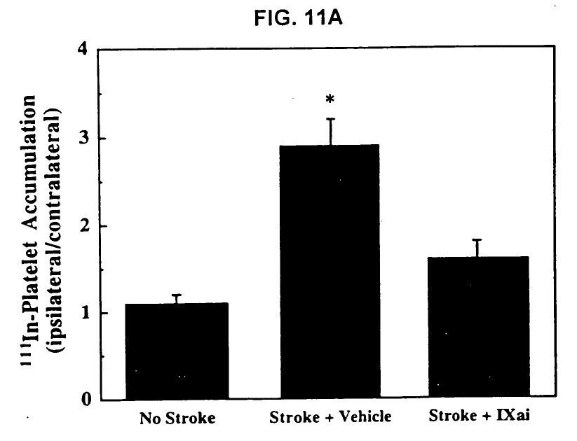

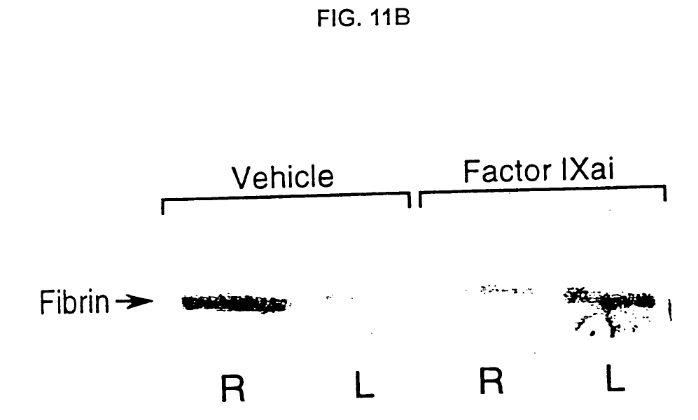

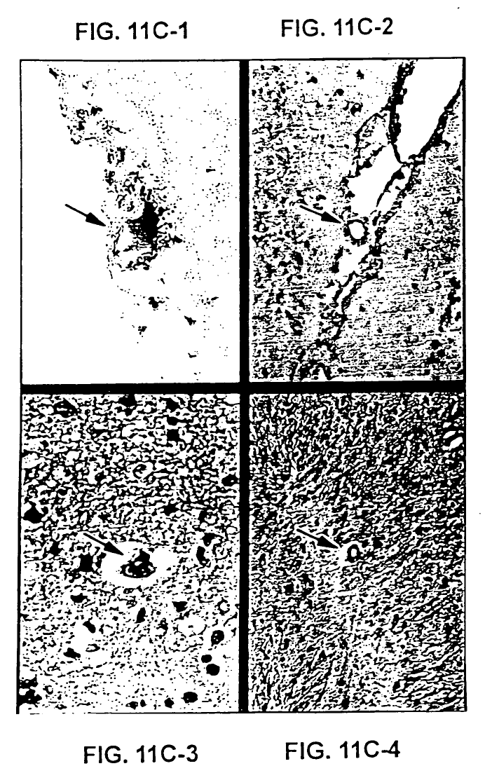

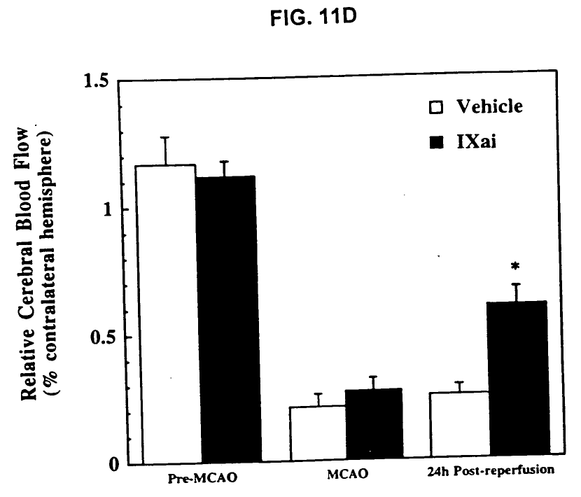

To create a stroke in a murine model, a suture is introduced into the cerebral vasculature so that it occludes the orifice of the right middle cerebral artery, rendering the subtended territory ischemic. By withdrawing the suture after a 45 minute period of occlusion, a reperfused model of stroke is created; mice so treated demonstrate focal neurological deficits as well as clear-cut areas of cerebral infarction. Because the occluding suture does not advance beyond the major vascular tributary (the middle cerebral artery), this model provides an excellent opportunity to investigate “downstream” events that occur within the cerebral microvasculature in response to the period of interrupted blood flow. Using this model, the role of microvascular thrombosis was investigated as follows. To demonstrate that platelet-rich thrombotic foci occur within the ischemic cerebral hemisphere, ¹¹¹In-labeled platelets were administered to mice immediately prior to the introduction of the intraluminal occluding suture, to track their deposition during the ensuing period of cerebral ischemia and reperfusion. In animals not subjected to the surgical procedure to create stroke, the presence of platelets was approximately equal between the right and left hemispheres, as would be expected [FIG. 11A, left bar]. However, when animals were subjected to stroke (and received only vehicle to control for subsequent experiments), radiolabeled platelets preferentially accumulated in the ischemic (ipsilateral) hemisphere, compared with significantly less deposition in the contralateral (nonischemic) hemisphere [FIG. 11A, middle bar]. These data support the occurrence of platelet-rich thrombi in the ischemic territory. Another line of evidence also supports the occurrence of microvascular thrombosis in stroke. This data comes from the immunodetection of fibrin, using an antibody directed against a neoepitope on the gamma-gamma chain dimer of cross-linked fibrin. Immunoblots demonstrate a band of increased intensity in the ipsilateral (right) hemisphere of vehicle-treated animals subjected to focal cerebral ischemia and reperfusion [FIG. 11B, “Vehicle”]. To demonstrate that fibrin accumulation was due to the deposition of intravascular fibrin (rather than due to nonspecific permeability changes and exposure to subendothelial matrix), fibrin immunostaining clearly localized the increased fibrin to the lumina of ipsilateral intracerebral microvessels [FIG. 11C]. As an in vivo physiological correlate of microvascular thrombosis, relative cerebral blood flow was measured by laser doppler during the occlusive period as well as after stroke. These data [FIG. 11D, bars labelled “Vehicle”] show that the intraluminal suture technique significantly reduces ipsilateral cerebral blood flow during the occlusive period [FIG. 11D, middle panel]. Blood flow remains depressed even 24 hours after removing the intraluminal occluding suture [FIG. 11D, right panel], corresponsding to the platelet, fibrin immunoblot, and fibrin immunostaining data indicating the presence of postischemic microvascular thrombosis.

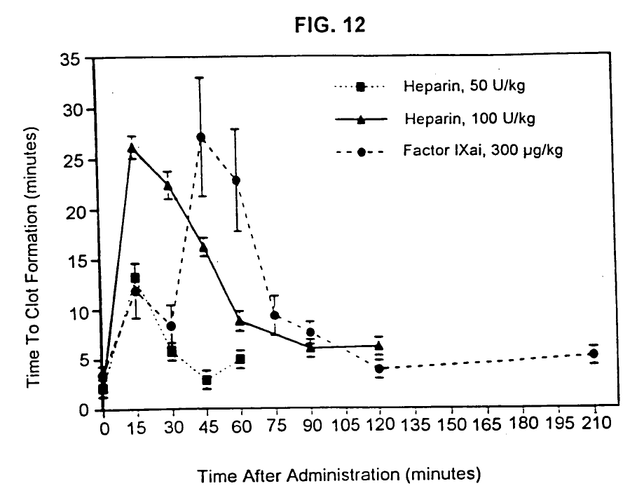

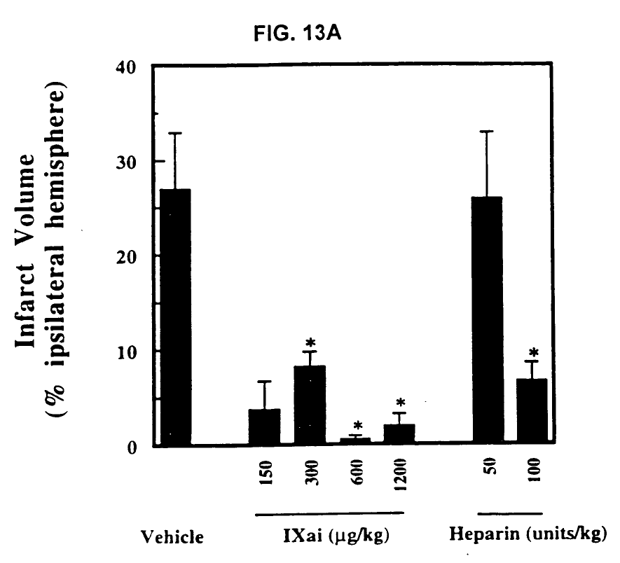

To help establish a functionally deleterious role of microvascular thrombosis in stroke, experiments were performed to test the effect of inhibiting assembly of the Factor IXa/VIIIa/X activation complex in vivo. This particular strategy was selected based upon the hypothesis that relatively selective inhibition of the intrinsic pathway of coagulation might inhibit intravascular thrombosis yet not impair tissue factor/VIIa-mediated extravascular hemostasis (and hence, may not increase intracerebral hemorrhage at clinically effective doses). Active-site blocked factor IXa (Factor IXai), formed by dansylation of the active site of Factor IXa, demonstrated antithrombotic potency similar to that of heparin when measured in a modified cephalin clotting time assay [FIG. 12], in which the activity of Factor IXa is a rate-limiting step in thrombus formation. To achieve this goal, Factor IXai was administered to mice immediately prior to stroke in various doses. When Factor IXai is administered to animals prior to introduction of the intraluminal occluding suture, there is a significant reduction in the accumulation of radiolabelled platelets in the ipsilateral hemisphere [FIG. 11A, rightmost bar], no apparent increase in the ipsilateral accumulation of fibrin [FIG. 11B, “Factor IXai”], as well as decreased evidence of intravascular fibrin by immunostaining. In addition, there is a significant increase in postischemic blood flow by this treatment, albeit not completely to preischemic levels [FIG. 11D].

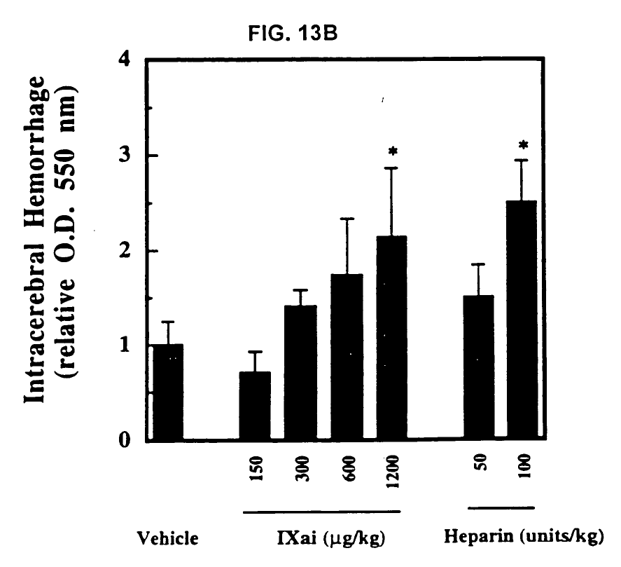

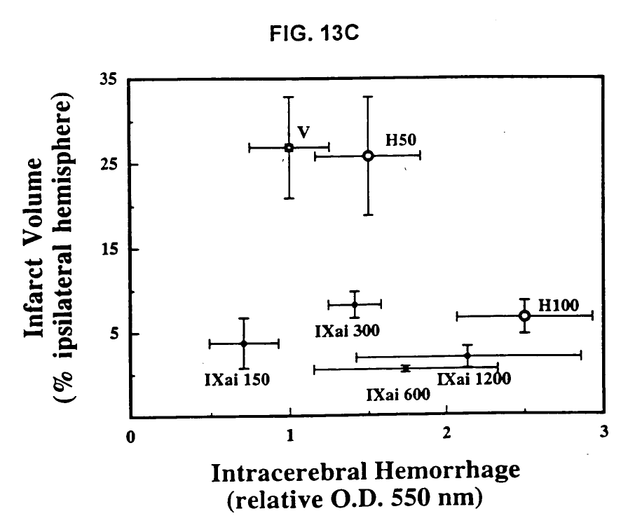

The clinical relevance of these observations is underscored by the striking ability of Factor IXai to reduce cerebral infarct volumes [FIG. 13A]. To test whether this infarct size-reducing property of Factor IXai was unique to this compound, or whether a nonspecific anticoagulant would also demonstrate efficacy in this regard, intravenous heparin was also examined at two doses. Only at the highest dose tested (100 U/kg) did heparin reduce cerebral infarct volumes, however, this was at the cost of a significant increase in intracerebral hemorrhage, measured with a recently validated spectrophotometric assay (17)[FIG. 13B]. In sharp contrast, Factor IXai caused an increase in ICH only at the highest dose tested, but did not do so at doses which demonstrated striking efficacy to reduce cerebral infarct volumes [FIG. 13B] Because a desirable therapeutic agent in stroke will not only reduce cerebral infarction volumes, but will also minimize ICH, the data shown in FIGS. 13A and 13B are displayed with infarct volumes plotted along the ordinate and intracerebral hemorrhage plotted along the abscissa [FIG. 13C]. As can be seen in the figure, Factor IXai appears to be therapeutically superior to heparin, because with heparin, it was a trade-off between infarct volume-reducing efficacy and increasing ICH, which was not the case with Factor IXai (minimized both infarction volumes and ICH). Pilot experiments in which tPA was administered to mice subjected to stroke resulted in reduced cerebral infarction volumes at the cost of increased ICH.

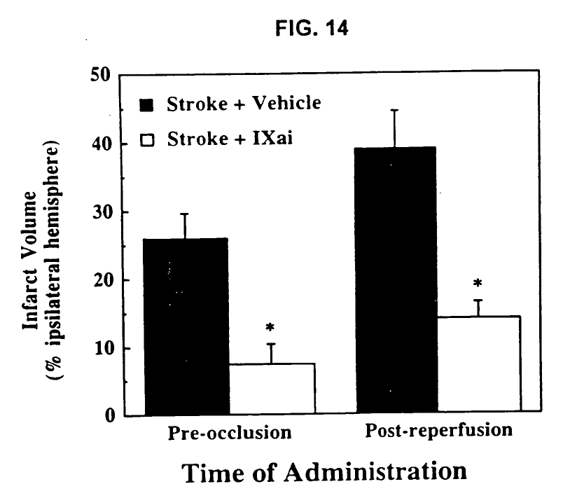

Because therapies directed at improving outcome from acute stroke must be given after clinical presentation, and because fibrin continues to form following the initial ischemic event in stroke, we tested whether Factor IXai might be effective when given following initiation of cerebral ischemia. Factor IXai given after middle cerebral artery occlusion (following removal of the occluding suture) provided significant cerebral protection judged by its ability to significantly reduce cerebral infarction volumes compared with vehicle-treated controls [FIG. 14].

Discussion

The data in these studies demonstrate clear evidence of intravascular thrombus formation (both platelets and fibrin) within the post-ischemic cerebral microvasculature. In fact, the ability of an anticoagulant such as Factor IXai to improve outcome even when given after the onset of the reperfusion phase suggests that the process of microvascular thrombosis is not limited to that which occurs during the major occlusive event. Rather, microvascular thrombosis appears to be a dynamic process which continues to evolve even after recanalazition of the major vascular tributary. The pathophysiological relevance of microvascular thrombosis in stroke is underscored by the ability of Factor IXai to reduce microvascular thrombosis (both platelet and fibrin accumulation are reduced, with an attendant increase in postischemic CBF) and to improve stroke outcome. These potent antithrombotic actions of Factor IXai are likely to be clinically significant in the setting of stroke, because Factor IXai not only reduces infarct volumes in a dose-dependent manner, but it does so even when given after the onset of stroke. In addition, at clinically relevant doses, treatment with Factor IXai does not cause an increase in ICH, making selective inhibition of Factor IXa/VIIIa/X activation complex assembly with Factor IXai an attractive target for stroke therapy in humans.

There are a number of reasons why targetted anticoagulant strategies might be an attractive alternative to the current use of thrombolytic agents in the management of acute stroke, because of their checkered success in clinical trials. Theoretically, an ideal treatment for acute stroke would prevent the formation or induce dissolution of the fibrin-platelet mesh that causes microvascular thrombosis in the ischemic zone without increasing the risk of intracerebral hemorrhage. However, thrombolytic agents which have been studied in clinical trials of acute stroke have consistently increased the risk of intracerebral hemorrhage (1-3,10). Streptokinase, given in the first several (<6) hours following stroke onset, was associated with an increased rate of hemorrhagic transformation (up to 67%); although there was increased early mortality, surviving patients suffered less residual disability. Administration of tissue-type plasminogen activator (tPA) within 7 hours (particularly within 3 hours) of stroke onset resulted in increased early mortality and increased rates of hemorrhagic conversion (between 7-20%), although survivors demonstrated less residual disability. In order to develop improved anticoagulant or thrombolytic therapies, several animal models of stroke have been examined. These models generally consist of the administration of clotted blood into the internal carotid artery followed by administration of a thrombolytic agent. In rats, tPA administration within 2 hours of stroke improved cerebral blood flow and reduced infarct size by up to 77% (19,20). In a similar rabbit embolic stroke model, tPA was effective at restoring blood flow and reducing infarct size, with occasional appearance of intracerebral hemorrhage (21,22). However, although there are advantages to immediate clot dissolution, there are several potential disadvantages of tPA; in murine models, tPA has been shown to directly mediate excitotoxic neuronal cell injury via extracellular tPA-catalyzed proteolysis of nonfibrin substrates (23-28). Moreover, animal studies (as well as the clinical trials of thrombolytic agents) indicate that there is an attendant increased risk of intracerebral hemorrhage with this therapeutic approach. In preliminary studies in which tPA was given after removal of the MCA occluding suture, doses of tPA which tended to reduce infarct volumes also increased the degree of ICH (Huang, Kim, Pinsky, unpublished observation).

Because of the usually precipitous onset of ischemic stroke, therapy has been targeted primarily towards lysing the major fibrinous/atheroembolic debris which occludes a major vascular tributary to the brain. However, as the current work demonstrates, there is an important component of microvascular thrombosis which occurs downstream from the site of original occlusion, which is likely to be of considerable pathophysiological significance for post-ischemic hypoperfusion (no-reflow) and cerebral injury in evolving stroke. This data is in excellent agreement with that which has been previously reported, in which microthrombi have been topographically localized to the ischemic region in fresh brain infarcts (29). The use of an agent which inhibits assembly of the Factor IXa/VIIIa/X activation complex represents a novel approach to limiting thrombosis which occurs within microvascular lumena, without impairing extravascular hemostasis, the maintenance *of which may be critical for preventing ICH. In the current studies, treatment with Factor IXai reduces microvascular platelet and fibrin accumulation, improves postischemic cerebral blood flow, and reduces cerebral infarct volumes in the setting of stroke without increasing ICH.

The potency of Factor IXai as an anticoagulant agent stems from the integral role of activated Factor IX in the coagulation cascade. Not only does a strategy of Factor IXa blockade appear to be effective in the setting of stroke, but it also appears to be effective at preventing progressive coronary artery occlusion induced following the initial application of electric current to the left circumflex coronary artery in dogs (18).

The data which demonstrate that IXai given after the onset of stroke is effective leads to another interesting hypothesis, that the formation of thrombus represents a dynamic equilibrium between the processes of ongoing thrombosis and ongoing fibrinolysis. Even under normal (nonischemic) settings, this dynamic equilibrium has been shown to occur in man (30). The data in the current studies, which show that Factor IXai is effective even when administered after the onset of stroke, suggests that this strategy restores the dynamic equilibrium, which is shifted after cerebral ischemia to favor thrombosis, back towards a more quiescent (antithrombotic) vascular wall phenotype.

As a final consideration, even if thrombolysis successfully removes the major occluding thrombus, and/or anticoagulant strategies are effective to limit progressive microcirculatory thrombosis, blood flow usually fails to return to pre-ischemic levels. This is exemplified by data in the current study, in which although CBF is considerably improved by Factor IXai (which limits fibrin/platelet accumulation), CBF still does not return to preischemic levels. This data supports the existence of multiple effector mechanisms for postischemic cerebral hypoperfusion, including postischemic neutrophil accumulation and consequent microvascular plugging, with P-selectin and ICAM-1 expression by cerebral microvascular endothelial cells being particularly germane in this regard (11,12). When looked at from the perspective of leukocyte adhesion receptor expression, even when these adhesion receptors are absent, CBF levels are improved following stroke compared with controls but do not return to preischemic levels. Taken together, these data suggests that microvascular thrombosis and leukocyte adhesion together contribute to postischemic cerebral hypoperfusion.

In summary, administration of a competitive inhibitor of Factor IXa, active-site blocked Factor IXa, represents a novel therapy for the treatment of stroke. This therapy not only reduces microcirculatory thrombosis, improves postischemic cerebral blood flow, and reduces cerebral tissue injury following stroke, but it can do so even if given after the onset of cerebral ischemia and without increasing the risk of ICH. This combination of beneficial properties and relatively low downside risk of hemorrhagic transformation makes this an extremely attractive approach for further testing and potential clinical trials in human stroke.

### REFERENCES

1. The National Institute of Neurological Disorders and Stroke rt-PA Stroke Study Group (1995) N. Engl. J. Med. 333:1581-1587.
2. Hacke, W., et al. (1995) J. A. M. A. 274 (13):1017-1025.
3. del Zoppo, G. J. (1995) N. Engl. J. Med. 333(13):1632-1633.
4. Duke R J, et al. (1996) Annals of Internal Medicine 105:825-828.
5. Haley E C Jr., et al. (1988) Stroke 19:10-14.
6. Slivka A, and Levy D. (1990) Stroke 21:1657-1662.
7. Ramirez-Lassepas M, et al. (1986) Arch. Neurol. 43:386-390.
8. Bogousslavsky J. and Regli F. (1985) Acta Neurol Scand 71:464-471.
9. Cerebral Embolism Study Group (1984) Stroke 15:779-789.
10. Hommel, M., et al. (1996) N. Engl. J. Med. 335:145-150.
11. Connolly, E. S. Jr., et al. (1996) J Clin Invest 97:209-216.
12. Connolly, E. S. Jr., et al. (1997) Circ. Res. 81:304-310.
13. Connolly, E. S. Jr., et al. (1996) Neurosurg. 38(3):523-532.
14. Naka, Y., et al. (1995) Circ. Res. 76:900-906.
15. Lawson, C. A., et al. (1997) Journal of Clinical Investigation 99:1729-1738.
16. Lahiri, B., et al. (1981) Thromb. Res. 23:103-112.
17. Choudhri, T. F., et al. (1997) Stroke 28:2296-2302.
18. Benedict, C. R., et al. (1991) J. Clin. Invest. 88:1760-1765.
19. Papadopoulos, S. M., et al. (1987) J Neurosurg 67:394-398.
20. Overgaard, K., et al. (1993) Neurol Res 15:344-349.
21. Carter, L. P., et al. (1992) Stroke 23:883-888.
22. Phillips, D. A., et al. (1990) Stroke 21:602-605.
23. Tsirka, S. E., et al. (1997) Proceedings of the National Academy of Sciences USA 94:9779-9781.
24. Tsirka, S. E., et al. (1997) The Journal of Neuroscience 17:543-552.
25. Korninger, C. and D. Collen (1981) Thrombosis Haemostasis 46:561-565.
26. Tsirka, S. E., et al. (1996) Nature 384:123-124.
27. Sappino, A.-P., et al. (1993) Journal of Clinical Investigation 92:679-685.
28. Tsirka, S. E., et al. (1995) Nature 377:340-344.
29. Heye, N., et al. (1992) Acta Neurologica Scandinavica 86:450-454.
30. Nossel, H. L. (1981) Nature 291:165-167.

### EXAMPLE 5

### Microvascular Thrombosis as a Pathophysiolocical Mechanism in Ischemic Stroke and Use of Active-site Blocked Factor IX as a Novel Treatment

Ischemic stroke is the third leading cause of death in the United States. Current treatments aim to reestablish perfusion to ischemic brain by thrombolysis, however, they can increase the risk of hemorrhage, particularly in the setting of ischemia. Studies of acute stroke thus far have focused on ischemia associated with thromboembolic occlusion of cerebral vascular tributary. It is hypothesized, however, that ischemic injury is not produced solely by the original occlusion, but that the initial ischemic event modifies the microvasculature to trigger further local/microvascular thrombosis which contributes to post-ischemic hypoperfusion (no-reflow). An ideal treatment would overcome post-ischemic microvascular thrombosis and allow reperfusion without increasing the risk of hemorrhage.

Materials and Methods: Post-ischemic microvascular thrombosis in a murine model of ischemic stroke in which the right middle cerebral artery (MCA) is transiently occluded for 45 minutes was studied. The role of platelets and fibrin were investigated using ¹¹¹-Indium-labeled platelets and fibrin immunostaining. We studied the efficacy of a novel anticoagulant, active-site blocked factor IX (IXAI, 150-300 μg/kg IV), which inhibits the Factor IXA/VIIIa/X activation complex. Outcome indices were platelet accumulation (measured as an ipsilateral to contralateral ratio), relative cerebral blood flow measured by laser doppler (CBF, ratio of ipsilateral to contralateral hemispheric flow), and infarct volume (Inf Volume, % ipsilateral hemisphere by triphenyltetrazolium chloride staining). In addition, intracerebral hemorrhage (ICH) was quantified in homogenized brain tissue using a method which we developed and validated, based on the conversion of hemoglobin to cyanomethemoglobin (OD measured at 550 nm; the amount of intracerebral blood is linearly related to OD).

Results:

TABLE III

| | | | | Inf | |
|---|---|---|---|---|---|
| | Platelets | Fibrin | CBF | Volume | ICH |
| No Stroke | 1.1 ± 0.1 | 0 | 110 ± 8 | 0.0 ± 0 | 0.07 ± 0.0 |
| (n = 11) | | | | | |
| Stroke + | 2.9 ± 0.3* | ++ | 37 ± 5* | 26 ± 3.7* | 0.15 ± 0.04* |
| Placebo | | | | | |
| (n = 62) | | | | | |
| Stroke + | 1.6 ± 0.2* | + | 61 ± 6** | 7.4 ± 3.0** | 0.12 ± 0.02 |
| IXai | | | | | |
| (n = 48) | | | | | |

(Results are expressed as means ± SEM.
*p < 0.05 vs. no stroke,
**o < 0.01 vs. stroke + placebo)

These data, along with immunohistochemical evidence of intravascular fibrin only in the ischemic hemisphere, show that thrombus accumulates within the post-ischemic cerebral microvasculature. Furthermore, IXai reduces both this platelet and fibrin accumulation, improves CBF, and reduces infarct volumes in a dose-dependent manner. The advantage of IXai in treating stroke without increasing ICH was shown in experiments where it did not increase ICH when compared with controls (0.12±0.02 vs. 0.15±0.04, p=NS). The benefit of IXai was also observed when given after the onset of stroke (placebo infarct volume 39±5.5% vs. IXai 14±2.4%, p<0.05)

Conclusions: In ischemic regions of brain, platelets and fibrin accumulate to form microvascular thrombosis, contributing to post-ischemic hypoperfusion (no-reflow). Treatment with IXai reduces platelet and fibrin accumulation, improves CBF, and reduces infarct volume without increasing ICH.

### EXAMPLE 6

### Active-Site Blocked Factor Ixai: An Alternative Anticoagulant for Use in Hemodialysis

Significant bleeding complications during hemodialysis (HD) in high-risk patients (GI/intracerebral hemorrhage) have been reported with an incidence as high as 26%. Patients with increased risk of bleeding as well as those with specific contraindications to heparin would greatly benefit from an alternative anticoagulant for use in HD. Active-site blocked factor IXA (Ixai) has previously been shown to selectively block the intrinsic/contact mediated pathway of coagulation in the setting of contact of blood with an extracorporeal circuit, while maintaining extravascular/tissue factor-mediated hemostasis. In order to investigate the use of this novel anticoagulant strategy in the setting of HD and chronic uremia, obstructive renal failure was induced in 11 female mongrel dogs by bilateral ureteral ligation through a midline laparotomy. Renal failure, as indicated by a rise in BUN>65 mg/dl, was reliably induced within 48 hours at which time the animals underwent standard HD using COBE Centrysystem 3 equipped with 300 HG hemodialyzers and standard bicarbonate dialysate (BiCart). Venovenous HD lasted for three hours and was performed on three consecutive days at flows of 300-350 ml/min. HD was successfully completed using Ixai (400-460 μg/kg given at 0 min & 90 min) or standard heparin with equivalent efficacy as reflected by the urea reduction ratio (74.86%±3.43% vs. 78.16%+2.49%, p=43). There was no evidence of gross clot formation in the tubing or resultant increase in circuit pressure. Analysis of data from incisional wound models at 15 min suggested a decreased bleeding tendency in IXai treated animals as compared to those treated with heparin (0.05±0.11 gm vs. 0.38±0.17 gm closed wound, p=0.004; 4.59+1.74 gm vs. 8.75±2.09 gm open wound, p=17). IXai, a selective anticoagulant which confers extracorporeal circuit anticoagulation without compromising extravascular hemostasis, may therefore represent a novel alternative anticoagulant strategy for use in chronic HD.

### EXAMPLE 7

### Role of Factor IXai in Pulmonary Ischemia and Reperfusion and Role of Factor IXai as an Adjunct to tissue-type plasminogen activator (tPA) in stroke

(1) Factor IXai can be effective at lower doses with the lower doses being less likely to cause intracerebral hemorrhage. This Example includes data regarding the dose response range of Factor IXai with respect to its effect on clotting time in the modified cephalin clotting time assay. The dose/response data with respect to intracerebral hemorrhage can be found in the data provided in Example 4.

(2) Factor IXai is effective in other types of ischemia (and reperfusion. New data shown in this example show that when the lungs are subjected to ischemia and reperfusion (by cross-clamping their blood supply, waiting a bit, and then releasing the clamp), Factor IXai is protective. Both the lung function (oxygenation of blood) and survival of the animal which had received Factor IXai was better than that seen in vehicle-treated animals.

(3) Factor IXai may be effective after the thrombotic event; i.e., it is effective when given after stroke, not just beforehand. This data can be found in the information hereinabove in Example 4.

(4) Factor IXai may be useful to lower the dose of thrombolytic therapy necessary to achieve reperfusion (for instance, in heart attacks, stroke, pulmonary emboli, etc.). The data which shows this point is in Table IV hereinbelow. In a stroke model, a dose of tissue-type plasminogen activator (an example of a commonly used thrombolytic agent) which itself did not protect the brain in stroke was given in combination with a dose of Factor IXai which was too low by itself to confer protection; however, the combination was significantly protective (reduced cerebral infarction volume) without causing any excess in intracerebral hemorrhage.

Role of Factor IXai in Pulmonary Ischemia and Reperfusion:

Seven C57BL mice (male 25 gm) were anesthetized with ketamine and xylazine, and a bilateral thoracotomy was performed using a clam-shell incision. A loose suture was placed around the right pulmonary artery, and the left pulmonary hilum was exposed. An intravenous injection was given (0.3 mL of either saline [control, n=4] or Factor IXai [300 μg, n=3]. After 3 minutes, the left pulmonary hilum (pulmonary artery, vein, and bronchus) was cross-clamped for 1 hour to create ischemia, after which the cross-clamp was released and the left lung reperfused and ventilated for 1 hour. After this reperfusion period, the loose suture around the right pulmonary artery was tightened, so that the animal's arterial oxygenation and survival depended solely on the function of the postischemic left lung. The data revealed that in the control group, the mean arterial oxygenation was 66 mm Hg, whereas in the Factor IXai-treated group, it was 120 mm Hg. Factor IXai also improved survival, in that 100% of control animals failed to survive the right pulmonary artery ligation procedure (mean time to death, 10 minutes), whereas ⅔ of the Factor IXai-treated animals survived for 30 minutes (at which time they were sacrificed for arterial blood gas analysis). Taken together, these data show that Factor IXai can protect against ischemia reperfusion injury in this model, and extend the previous data which showed that Factor IXai was protective after middle cerebral artery ischemia and reperfusion.

Role of Factor IXai as an Adjunct to Tissue-Type Plasminogen Activator (tPA) in Stroke:

For these data, 17 mice were used, and subjected to middle cerebral artery occlusion (45 minutes) and reperfusion as described hereinabove. Because Factor IXai by itself has been shown to have a dose-related cerebroprotective effect in stroke, a dose was chosen which we had previously shown to be below the protective threshold (50 μg/kg). In the experimental group, mice were given 50 μg/kg of Factor IXai preoperatively, and tPA was given immediately after withdrawal of the occluding suture at a dose of 0.5 mg/kg. Either of these agents when given by themselves at these low doses did not confer cerebral protection. However, compared to control animals which received vehicle alone (n=7), when tPA 0.5 mg/kg and Factor IXai (50 μg/kg) (n=10) were combined, there was significant protection; relative cerebral blood flows are expressed as an ipsilateral/contralateral blood flow ratio (×100), Infarct volumes are expressed as the percent of the ipsilateral hemisphere which was infarcted, and intracerebral hemorrhage was recorded as the optical density at 550 nm (higher numbers mean more hemorrhage, using our recently validated spectrophotometric method for quantifying intracerebral hemorrhage). **=p<0.001 vs. control.

TABLE IV

| | | | Interacerebral |
|---|---|---|---|
| Relative cerebral blood flow | | Infarct Volume | Hemorrhage |
| Control | 39 ± 6.4% | 29.6 ± 8.4% | 0.112 ± 0.013 |
| IXai + tPA | 72 ± 4.1%*** | 10.0 ± 2.6%*** | 0.110 ± 0.014 |

We conclude that administration of Factor IXai even at low doses can make tPA effective and cerebroprotective, at doses of tPA which otherwise showed no beneficial effects in previous experiments. Note that the combination treatment did not increase the degree of intracerebral hemorrhage.

### EXAMPLE 8

### Inhibition of Factor IXa-Dependent Coagulation Ameliorates Murine Pulmonary Ischemia/Reperfusion Injury

Introduction

Ischemia/reperfusion induces endothelial cell injury and microvascular thrombosis, which exacerbates tissue injury. Anticoagulation with heparin may be suboptimal in certain ischemic settings, especially when operative intervention is required, due to the increased propensity for bleeding. Furthermore, heparin-mediated immunologic reactions may contraindicate its use. The current studies test the hypothesis that inhibiting coagulation at the level of Factor IXa may be particularly useful in a surgically-induced model of lung ischemia, by reducing postischemic thrombosis, improving postischemic blood flow, and having relatively little effect on extravascular hemostasis. Using a murine model of pulmonary ischemia (1 hr) and reperfusion (3 hrs), the effects of vehicle (control) and heparin were compared with those of active-site blocked Factor IXa (Factor IXai), which competitively interferes with Factor VIIIa/IXa/Xa complex assembly and thereby inhibits the intrinsic pathway of coagulation. In a standardized model of surgical bleeding, tail vein bleeding times were unaltered by Factor IXai, but were significantly increased (1.82-fold, p<0.05 vs controls) by heparin. Under control conditions, microvascular thrombosis of the postischemic lung was demonstrated by increased fibrin formation in immunoblots of lung tissue extracts, increased deposition of ¹¹¹In-labeled platelets, and predominant intravascular localization of fibrin by immunohistochemistry. Both heparin and Factor IXai inhibited microvascular thrombosis, but heparin did so at the expense of increasing the volume of blood loss in the surgical field by 13.7 times (p<0.001 for H-300 vs control, p=NS for Factor IXai-300 vs control) and increasing intraparenchymal hemorrhage at the 600 U/kg dose. To assess the functional impact of anticoagulant strategies on the postischemic lung, the nonischemic (right) lung was effectively removed from the circulation 3 hours after left lung reperfusion by right pulmonary arterial ligation, so that survival was wholly-dependent on function of the postischemic left lung. Although heparin failed to improve survival, preischemic administration of Factor IXai improved-survival 3-fold (p<0.05 vs control). In addition to relative absence of surgical bleeding and intraparenchymal hemorrhage, Factor IXai administration was associated with reduced accumulation of platelets in postischemic tissue and the lowest IL-1b levels of any group. These data indicate that inhibiting Factor IXa-mediated coagulation can prevent microvascular thrombosis without impairing extravascular hemostasis and yield functional benefits in a surgically-induced model of lung ischemia.

When vital organs are exposed to a period of ischemia, vascular homeostatic mechanisms are disrupted, which can tip the normally preponderant anticoagulant milieu of the endovascular wall to one which favors activation of coagulation and accretion of thrombus. As microvascular thrombosis can impede the return of blood flow even when perfusion pressure is normalized, this can exacerbate and create ongoing tissue damage. In the brain, postischemic microvascular thrombosis contributes significantly to ischemic cerebral tissue damage (1). In the heart, postischemic no reflow has been documented even following relief of the major vascular obstruction. The lungs are a particularly vulnerable tissue in terms of their response to ischemic injury, and even relatively minor interruptions of blood flow can lead to regional pulmonary infarcts; in situ thrombosis of pulmonary veins is recognized to occur and contribute to primary lung graft failure after transplantation (2). Because of the need to limit ongoing thrombosis in a number of ischemic syndromes, heparin has been employed as a standard anticoagulant (3). However, as frank ischemia decreases the integrity of small vessels, which become increasingly fragile in the first several days after a profound ischemic insult (4), conventional anticoagulant strategies carry with them the risk of increasing hemorrhage in the postischemic tissue. Anticoagulant strategies may therefore be a double-edged sword, because progressive microvascular damage is associated with increased intraparenchymal hemorrhage following reperfusion (5). Furthermore, when ischemia occurs simultaneously with the need for a major operative intervention, when hemostatic control is of the utmost importance, conventional anticoagulant strategies may be unwise.

Although heparin has proven clinical utility in treating certain thrombotic disorders, such as unstable angina, pulmonary and deep venous thrombosis, acute myocardial infarction, heparin is not an ideal anticoagulant for several reasons. Its anticoagulant effects are unpredictable, and heparin-induced thrombocytopenia limits its clinical utility in certain situations. Even if these downside risks are reduced through the use of low molecular weight heparin preparations, heparin exerts its anticoagulant actions at multiple sites in the coagulation cascade, including the distal common pathway by accelerating the action of antithrombin III; therefore, it inhibits coagulation initiated by either the intrinsic or the extrinsic effector limbs of the coagulation cascade. Theoretically, if one were to inhibit intrinsic coagulation with relative selectivity, then tissue Factor VIIa-mediated hemostasis may be preserved.

The current experiments were designed to test the hypothesis that inhibiting the activity of Factor IXa, which is critically positioned in the intrinsic coagulation pathway, may be of especial utility to inhibit pulmonary ischemia-induced thrombosis. Because there is a paucity of tissue factor in the intravascular space, as well as the presence of an inhibitor of the extrinsic/tissue factor pathway which circulates in plasma, it seems logical that the intrinsic system may play a pivotal role in intravascular clot formation (6). As cultured endothelium displays specific binding for Factor IXa, endothelial cell factor IX/IXa receptors would be strategically positioned to participate in the regulation of coagulation within the vasculature. Furthermore, as the interactions between activated factor IXa/VIIIa and factor X occur on the platelet surface (chiefly localized to the intravascular space), which greatly accelerates the activation of Factor X, the inhibition of Factor IXa activity appeared to be a logical target to inhibit intravascular coagulation that may occur due to an ischemic insult. On the other hand, from the perspective of surgical bleeding, this strategy also appeared likely to be relatively safe, because even in the absence of Factor IXa, tissue factor/VIIa may still activate Factor X and the final common pathway, hemostasis related to surgical procedures may remain relatively unimpaired. To test this hypothesis, a surgical model of pulmonary ischemia and reperfusion was tested in mice; the occurrence of ischemia-related thrombosis and its inhibition by heparin or factor IXa inhibition were studied, along with an examination of the role of these two strategies in terms of improving functional endpoints and inducing surgical bleeding.

Methods

Murine Pulmonary Ischemia/Reperfusion Model: In order to study the role of thrombosis and the effect of anticoagulation on pulmonary ischemia/reperfusion injury, a murine model of pulmonary ischemia/reperfusion was used. Ten-week-old male mice (25-30 g) were anesthetized using an intraperitoneal injection 0.3 mL of ketamine (3 mg/mouse) and xylazine (0.3 mg/mouse) prepared in physiological saline. Anesthesia was continued during surgery with 0.1 mL/hour of the solution containing ketamine 10 mg/mL and xylazine 1 mg/mL through a small tube which was placed into the peritoneum, with infusion controlled using a syringe pump (model 100 series, KD Scientific Inc. MA). Mice were intubated via a tracheal incision and ventilated with a Harvard ventilator (tidal volume 0.75 mL, respiratory rate 120/min) and underwent bilateral thoracotomy. Three different concentrations of heparin chloride (100, 300, or 600 U/kg), or three different concentrations of active site blocked factor IXa (see below; 150, 300, 600 μg/kg) prepared in 0.3 mL of lactated Ringer's were administered intravenously via the penile vein. The surgical procedure was continued as follows. The entire left pulmonary hilum was cross-clamped with a small vascular clamp for 1 hour, after which the cross-clamp was released, and the ischemic lung allowed to reperfuse for 3 hours; 0.2 mL of physiological saline was given intravenously via penile vein 2 hours after cross-clamping to maintain appropriate hydration. Microsurgical electrocautery was performed until there was no visible bleeding at the wound edges, and then two pieces of 2 in.×2 in. gauze were placed over the thoracic wound and covered with plastic (to prevent dessication) during observation. At three hours after reperfusion, the contralateral (right) pulmonary artery (PA) was ligated with 5-0 silk to exclude this lung from the circulation, so that the function of the postischemic left lung could be ascertained independent of the right lung. Survival was recorded at the 30-min time point after ligation of the right PA. As the mouse continued to be ventilated, death of the mouse was defined as a combination of (1) cessation of regular cardiac activity; (2) the apparent collapse of the left atrium; and (3) brief clonic activity indicating cessation of cerebral blood flow. Left lung tissue obtained from animals subjected to the survival protocol was harvested and analyzed for pulmonary hemorrhage as follows; 1.0 mL of physical saline was flushed into the pulmonary artery immediately before harvest, and a spectrophotometric assay for hemoglobin performed as described below. All other assays were performed using tissue obtained from a separate cohort of animals in whom survival was not assessed. This separate group of animals was subjected to similar surgical procedures up to the point of 3 hrs reperfusion, and was used to determine the cytokine profile and degree of fibrin deposition; for this additional cohort of animals, heparin (0.1 mL of 5000 Units/mL) was administered intravenously approximately 2-3 minutes before the 3 hour-reperfusion period (to limit postmortem fibrin deposition), a 700 μL sample of left ventricular blood obtained for subsequent cytokine measurements, and lung tissue harvested for immunohistological detection of fibrin. A separate group of animals was used for these experiments because it was felt (a priori) that systemic heparinization of animals to prevent postmortem thrombosis and left ventricular puncture to obtain blood would obfuscate the functional effects of the different treatment groups. These experiments were performed according to a protocol approved by the Columbia University Institutional Animal Care and Use Committee in accordance with guidelines of the American Association for the Accreditation of Laboratory Animal Care. spectrophotometric Assay for hemorrhage in lung tissue:

The hemoglobin content of lung tissue which had been subjected to the experimental procedures described above was quantified with a spectrophotometric assay for hemoglobin in tissue (7). Whole left lung tissue was obtained from freshly killed control or experimental animals after being flushed with 1.0 mL of physical saline to remove residual blood in the pulmonary vessels. Distilled water (1 mL) was added to each lung, followed by homogenization for 30 seconds (Brinkmann Instruments, Inc., Westbury N.Y.), sonication on ice with a pulse ultrasonicator for 1 min, and centrifugation at 13,000 rpm for 30 min. After the he moglobin-containing supernatant was collected, 80 μL of Drabkin's reagent (Sigma Diagnostics; K₃Fe(CN)(200 mg/L, KCN 50 mg/L, NaHCO₃ 1 g/L, pH 8.6) (8) was added to a 20 μL aliquot and allowed to stand for 15 min. at room temperature. This reaction converts hemoglobin to cyanomethemoglobin, whose concentration can then be assessed by the OD of the solution at 550 nm wavelength. To validate that the measured absorbance following these procedures reflects the amount of hemoglobin in tissue, known quantities of bovine erythrocyte hemoglobin (Sigma) were analyzed with similar procedures alongside every lung tissue assay.

Measurement of surgical bleeding: The degree of surgical blood loss was quantified by measuring the amount of blood absorbed into two 2″×2″ gauze pads, which had been uniformly placed over the surgical wound for a uniform duration (4 hrs total: 1 hr ischemia+3 hrs reperfusion), during which time they were left unmanipulated except for a brief period every hour during which any sites of visible bleeding were recauterized. After 4 hours (1 hr ischemia+3 hrs reperfusion), the gauze pads were removed and their hemoglobin content quantified.

Following removal, the gauze was kept at −80° C. until measurement. Hemoglobin was eluted from the gauze by immersing the gauze in 5 mL of physiological saline, followed by 2 minutes of gentle agitation, after which the eluate was recovered. A plasma hemoglobin diagnostic kit (Sigma Diagnostics) was used to measure the hemoglobin concentration in the eluate, according to procedures provided by the manufacturer. The calorimetric determination of hemoglobin is based upon the ability of hemoglobin to accelerate the oxidation of benzidine by hydrogen peroxide. The resulting rate of color formation is proportional to the hemoglobin concentration of the test sample (9).

Measurement of bleeding time: Bleeding times were measured in mice that were not subjected to experimental manipulation other than by receiving vehicle, heparin, or Factor IXai prepared in physiological saline and administrated intravenously 5 min before the experiment. After anesthesia, a standardized incision was made on the central tail vein, and the tail was then immersed in physiological saline at 37.5° C. Time was recorded from the moment blood was observed to emerge from the wound until cessation of blood flow (1,10).

Western immunoblotting for fibrin: Lung tissue, harvested as above, was placed in buffer (Tris. 0.05 M, NaCl 0.15 M, heparin 500 U/mL, finally pH 7.6) on ice and homogenized (Brinkmann Instruments, Inc.). Plasmin digestion was performed by a modification of the methods of Francis (11), as previously described (12). Human plasmin (0.32 U/mL, Sigma Chemical Co.) was added to the tissue homogenate, followed by agitation at 37° C. for 6 hours. More plasmin (0.32 U/mL) was then added, and samples were agitated for an additional 2 hours. The mixture was centrifuged at 2300 g for 15 min and the supernatant which included fibrin was collected and kept at −80° C. until measurement. As a positive control, mouse fibrinogen (2.5 mg in 0.25 mL: Sigma Chemical Co.) was clotted with human thrombin (4 U: Sigma Chemical Co) in tris-buffered saline (1.75 mL) in the presence of calcium chloride (0.013 mL of 2.5 M) for 4 hour at room temperature. Clotted fibrinogen sample was centrifuged for 5 min and the pellet was suspended in tris-buffered saline (1.0 mL)). Human plasmin (0.32 U/mL) was added and agitated at 37° C. for 6 hour. Additional plasmin (0.32 U/mL) was added and samples were agitated at 37° C. for 2 more hours, after which no visible thrombus was present. As a negative control, unclotted mouse fibrinogen was processed in an identical manner. Protein concentration of plasmin-treated lung supernatants and plasmin-treated unclotted and clotted fibrinogen solutions were measured by the Bradford method (13) before loading the gel. Samples were boiled for 3 min. under reducing conditions, loaded onto a SDS-polyacrylamide gel (7.5% reduced gel; 25 μg protein per lane), and subjected to electrophoresis. Samples were electrophoretically transferred to nitrocellulose, and blots were reacted with a murine monoclonal anti-fibrin IgG1 (Biodesign International, ME) that had been prepared with human fibrin-like beta peptide as immunogen (14). The cross-reactivity of this antibody with murine fibrin was confirmed by blotting with the positive (murine fibrin) and negative (murine fibrinogen) controls prepared as described above. Secondary detection of sites of primary anrtibody localization was accomplished using a horseradish peroxidase-conjugated goat anti-mouse IgG (Fc) (Sigma Chemical Co). Final detection of bands was performed using the enhanced chemiluminescent Western blotting system (Amersham International, Buckinghamshire, England).

Immunohistochemistry: Lung tissue, harvested as above, was used to detect the fibrin by immunostaining. Left lung was put in OCT compound and immerse that sample in the liquid nitrogen to fix for the frozen section. This section was immunostained using the same first antibody as that used for western blotting, which was already checked that it had a cross-reactivity with murine fibrin. Primary antibody was revealed using a goat anti-mouse IgG avidin-biotin-conjugated system, as per the manufacturer's instraction (Sigma), with tris buffer/naphthol AS-MX (Sigma FAST™ FAST RED) as chromogen.

Measurement of Platelet Accumulation: Platelet accumulation in the left lung exposed to ischemia/reperfusion was determined using ¹¹¹In-labeled murine platelets, prepared as previously described (15); Heparinized (500 U/mL) pooled blood (at least 5.0 mL total) was taken from strain-matched mice via left ventricular puncture. Platelets were isolated by differential centrifugation, first at 900 g for 5 min to obtain platelet-rich plasma, then this platelet-rich plasma was centrifuged at 2200 g for 15 min to form a platelet pellet, and this pellet was then suspended in 10 mL of acid/citrate/dextrose anticoagulant (ACD-A, containing 38 mmol/L citric acid, 75 mmol/L sodium citrate, and 135 mmol/L glucose). After three washes (2200 g for 15 min in 10 mL of ACD-A solution) the platelet pellet was suspended in 5 mL of ACD-A solution and centrifuged at 100 g for 5 min to get rid of contaminating red blood cells, and the supernatant was collected. ¹¹¹In oxyquinoline (70 μL of 1 mCi/mL, Amersham Mediphysics, Arlington Heights, Ill.) was added with gentle shaking for 30 min at room temperature, then washed three times at 2200 g for 15 min in 10 mL of ACD-A. The pellet was suspended in 0.6 mL of PBS, and the radioactivity of 5 μL of this solution was counted by gamma counting; for each experiment, a platelet suspension containing ≈1.0×10⁶ cpm/0.1 mL was injected intravenously into the mouse immediately before reperfusion. During periods in which the platelets were not used, they were maintained on ice, and gently vortexed prior to each use. Two nearly simultaneous experimental setups were performed for all of these experiments to minimize variations that may be caused by isotope decay or ex vivo changes in platelet reactivity. After 3 hours of reperfusion, 0.1 mL blood and left lung tissue were collected to determine platelet accumulation in the lungs normalized to that in the blood; this normalization process was performed so as to minimize variability in tissue counts that may occur as a result of unintentional variations in injection volumes, residual uninjected counts, or unrecognized tissue extravasation at the local injection site. Lung tissue platelet accumulation is expressed as the ratio of lung radioactivity to blood radioactivity.

Measurement of cytokine levels: A heparinized blood sample was taken from the recipient after three hours of pulmonary reperfusion as described above, centrifuged for 10 min. at 14,000 rpm, and the supernatant was recovered and frozen at −80° C. until the time of assay. Assays were performed for IL-1β, TNF, IL-6, and IL-10 using ELISA kits from Genzyme Diagnostics (Cambridge, Mass.), according to procedures suggested by the manufacturer. The lower limit for detection in these assays was 3 pg/mL for IL-1β, 5.1 pg/mL for TNFα, 3.1 pg/mL for IL-6, and 4 pg/mL for IL-10. Assays were performed in duplicate, with standards run each time the assay was performed.

Data Analysis: ANOVA was used to compare different conditions. Animal survival data was analyzed by contingency analysis using the Chi square statistic. Values are expressed as the mean±SEM, with differences considered statistically significant if P<0.05.

Results

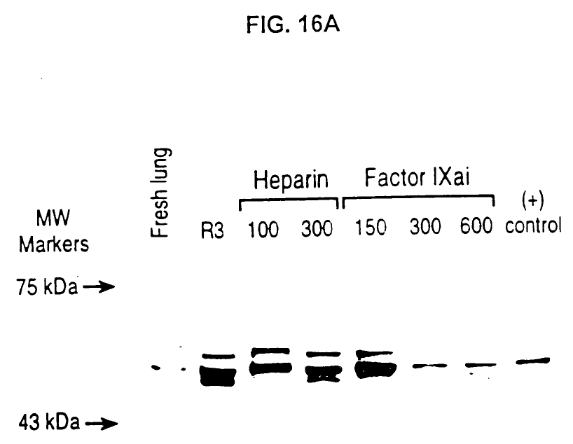

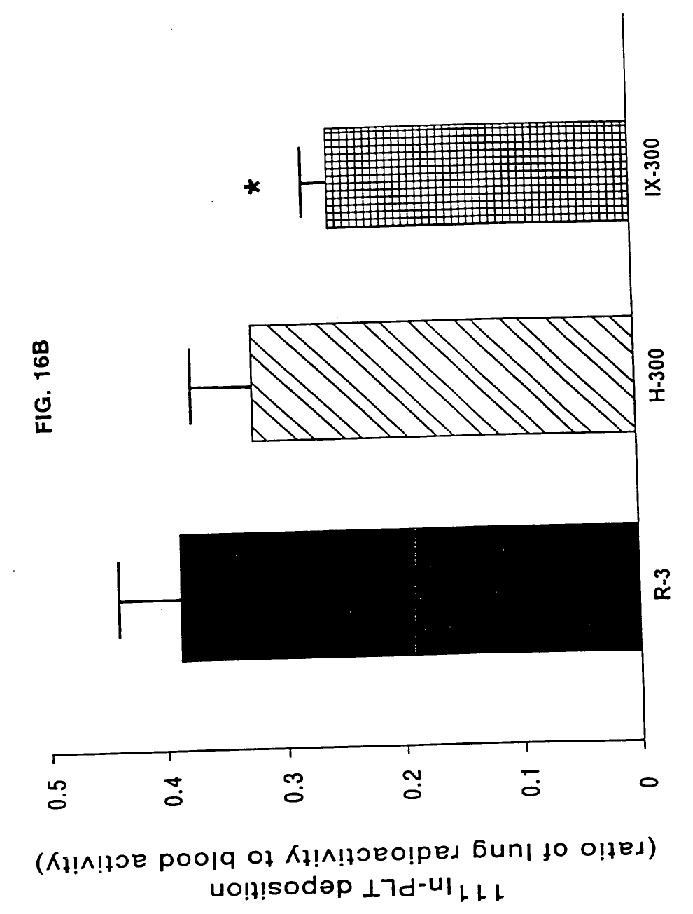

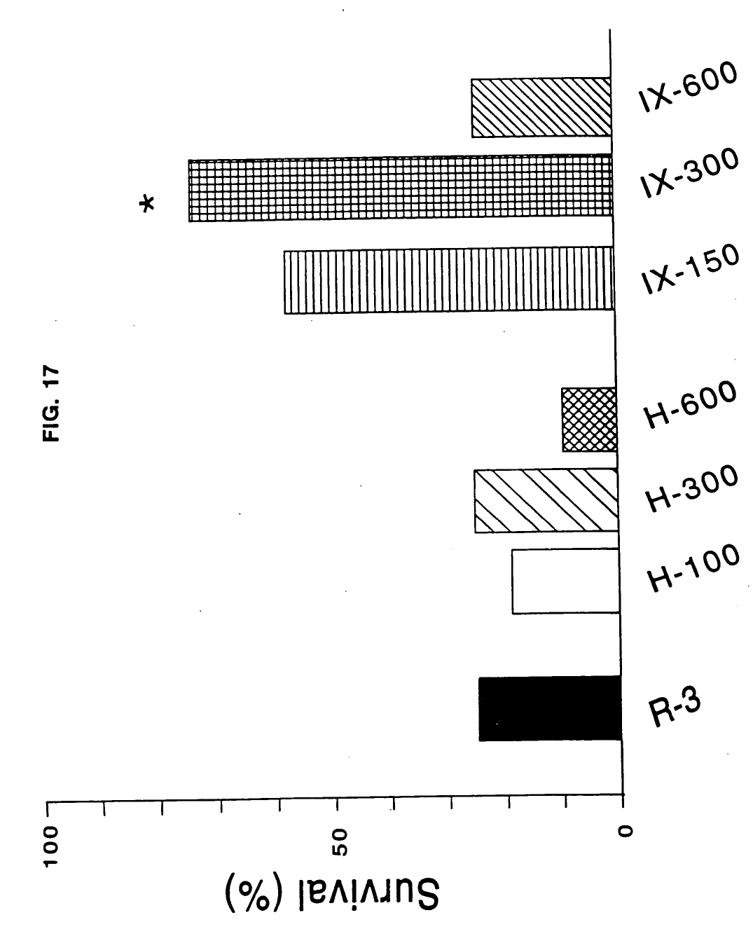

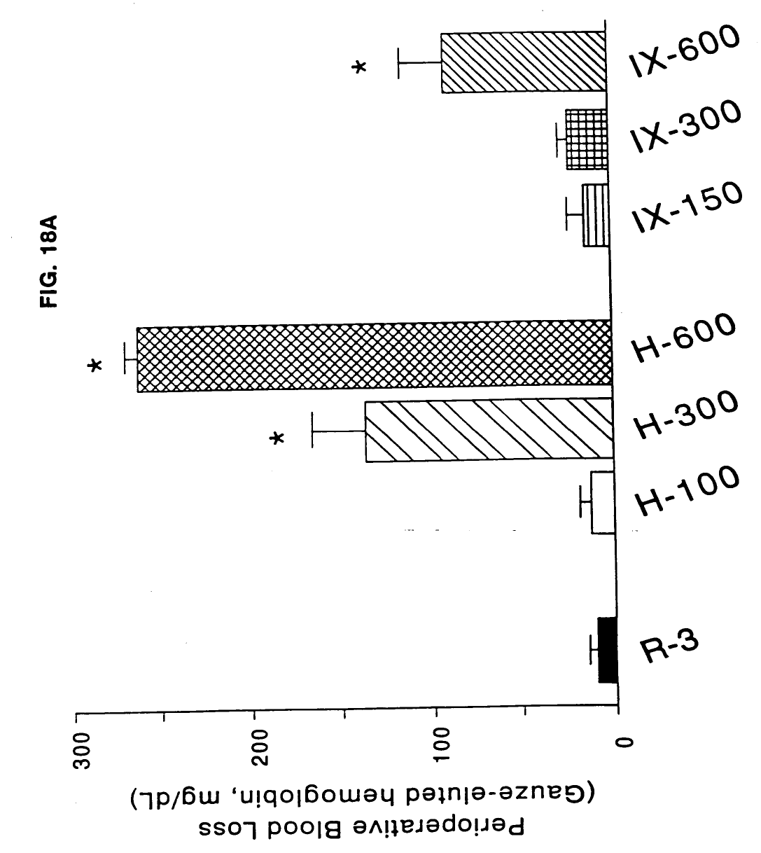

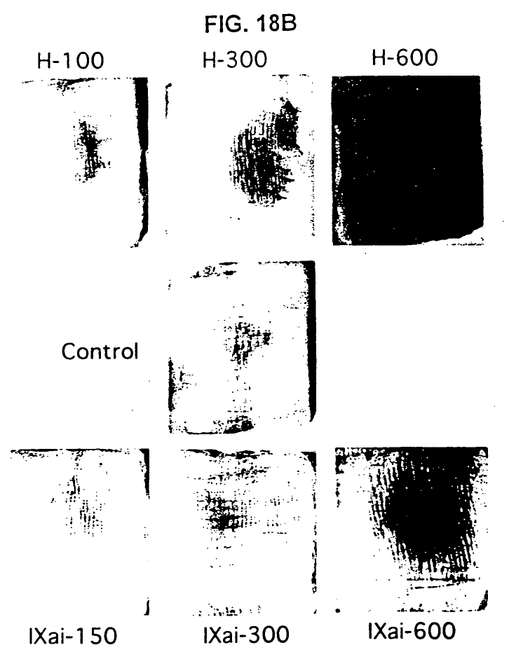

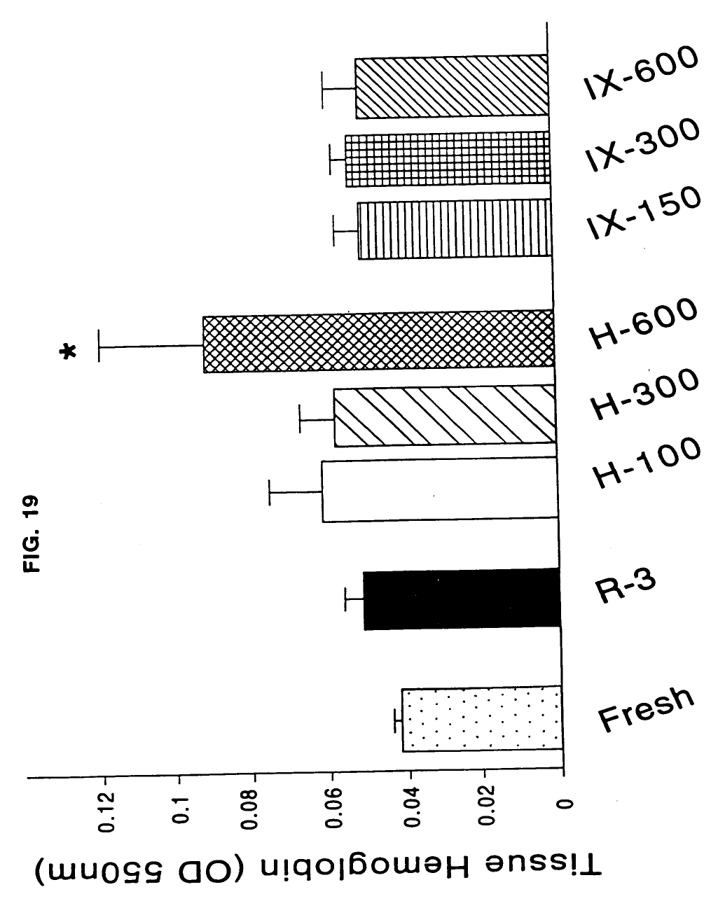

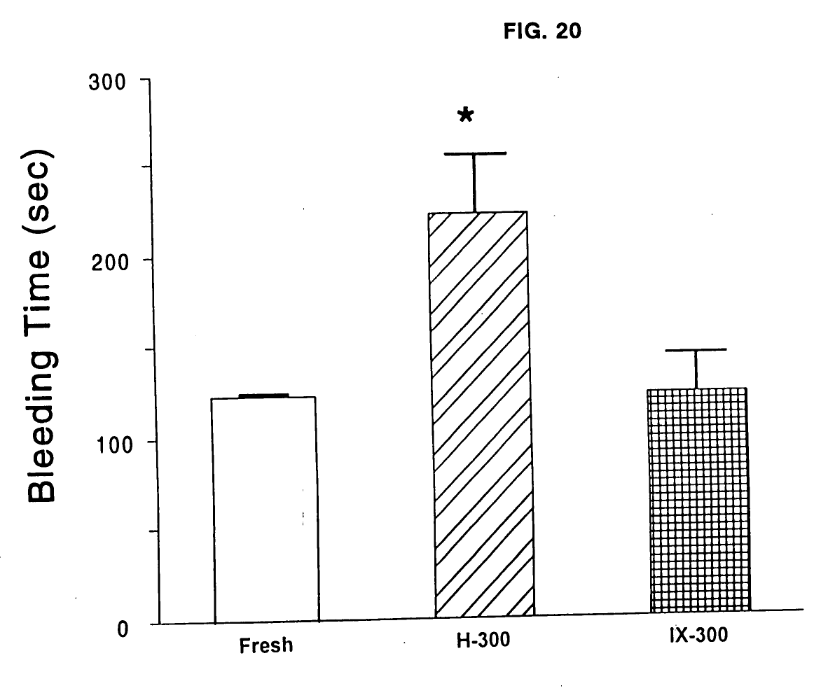

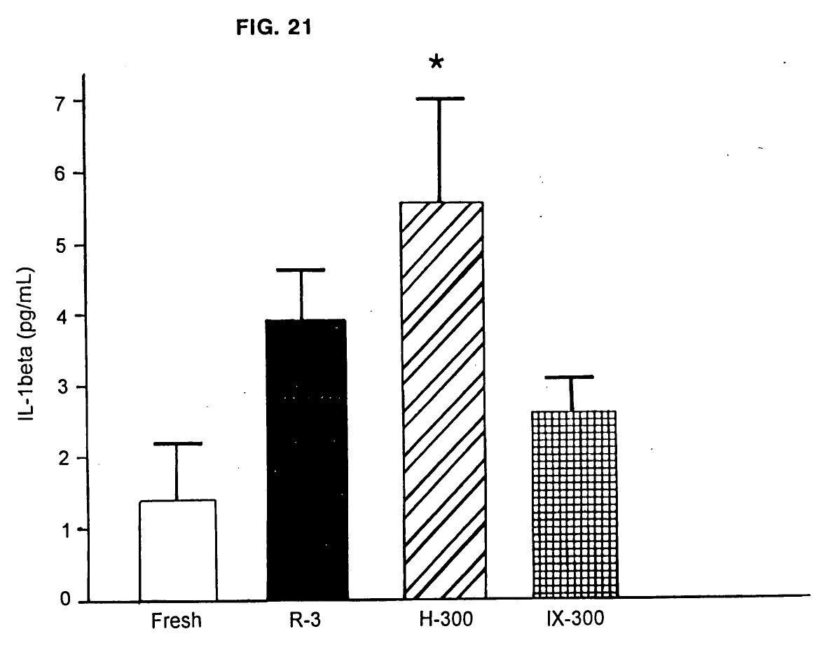

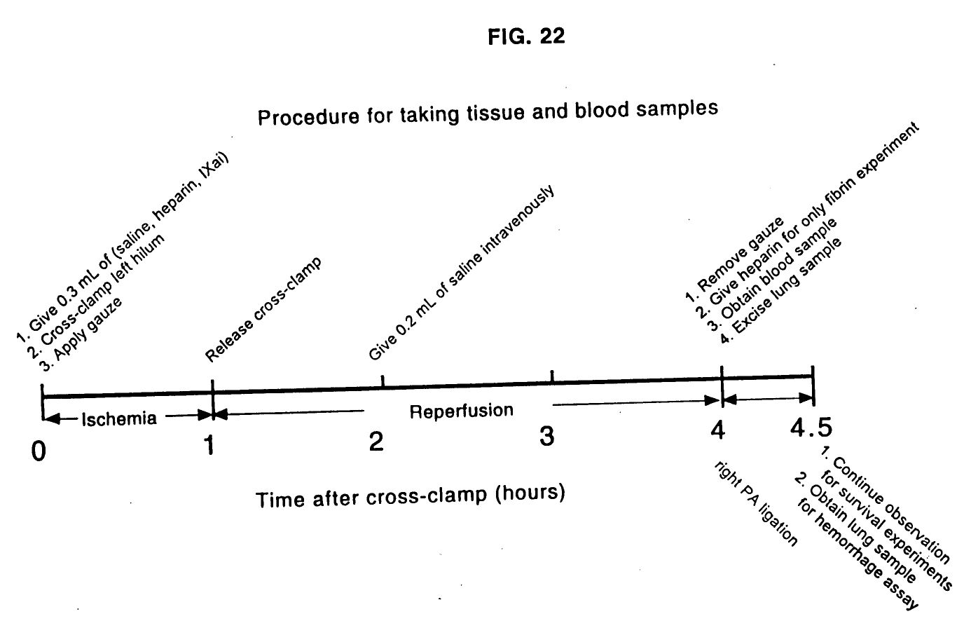

To determine whether pulmonary thrombosis contributes to the pathobiology of ischemia and reperfusion injury in the lungs, the extent of thrombosis in control lungs and those subjected to ischemia and reperfusion was studied by measuring fibrin accumulation and by measuring local platelet deposition. To dicide the lacalization of fibrin in the lung tissue, the immunostaining method was used. Fibrin was quantified as we have previously reported (12,16,17) by immunoblotting plasmin digests of pulmonary tissue. In these immunoblots, fibrin was judged to be present in the central band (the one of greatest intensity) which corresponds in molecular weight to the single band detected when fibrin prepared in vitro was used as a positive control. In these studies, lungs subjected to ischemia and reperfusion exhibited markedly increased fibrin accumulation (3.6-fold by densitometry) compared with that detected in fresh lung tissue [FIG. 16A]. Pretreating mice with either heparin or active site-blocked factor IXa significantly reduced the amount of fibrin which was detected by immunoblotting of postischemic lung tissue; the reduction of fibrin accumulation for both anticoagulants appeared to be dose-depedent, with higher pre-ischemic doses associated with lower levels of fibrin detected in postischemic tissue. As nascent thrombus grows by both fibrin accrual and by incorporation of platelets in the vicinity, additional studies were performed to determine the degree of sequestration of radiolabelled platelets in reperfused lung tissue. For these experiments, ¹¹¹In-labeled platelets were injected immediately before reperfusion, after which the lung was reperfused for three hours and then excised and the relative accumulation of radiolabelled platelets in the postischemic lung quantified. At intermediate doses of heparin and Factor IXai, only the Factor IXai was associated with a decrease in the relative accumualtion of platelets in the lung, althoguh there was a trend in that direction in the heparin group as well [FIG. 16B]. Immunohistochemistry demonstrated that the fibrin accumulation in the lung exposed to ischemia and reperfusion was within the pulmonary vasculature. When heparin and Factor IXai was administered at intermediate dose immediately before reperfusion, there are no apparent fibrin deposition in the vessels [FIG. 16C].

In separate experiments, the functional effects of the two anticoagulant strategies were examined in lung tissue using a stringent model wherein the contralateral (previously nonmanipulated) right lung was physically excluded from the circulation at the termination of the three hour left lung reperfusion period. Survival of the animal then depended entirely upon the function of the postischemic left lung. At the prespecified thirty minute time point following exclusion of the right lung from the circulation, treatment with heparin at any dose was observed to have no effect on survival compared with vehicle treated controls [FIG. 17]; in contrast, mice treated with an intermediate dose (300 μg/kg) of Factor IXai exhibited a much higher rate of survival. Although the highest dose of Factor IXai showed no effect on survival, there was a tendency (P=0.098) for animals pretrerated with 150 μg/kg of Factor IXai to have improved survival.

Although it was initially hypothesized that intervening in coagulation would have a positive impact of survival after the surgical procedure, we were surprised to have seen no beneficial therapeutic effect with heparin, and a loss of the protective effect when the highest dose of Factor IXai was examined. This data was in contrast to the dose-depedendent inhibition which was observed for both agents in terms of their abilities to inhibit fibrin formation in the lungs. These data suggested to us that an alternative, competing mechanism may have been responsible for the death of animals (or the lack of apparent protection by heparin or the highest dose of Factor IXai). To investigate this possibility, the degree of surgical blood loss with both therapies was objectively quantified. Two gauze pads were placed in a standardized way over the surgical wound after hemostasis was initially achieved under visual inspection. Every hour thereafter, the gauze pad was gently lifted up and any sites of visible bleeding recauterized. After 4 hours (1 hr ischemia+3 hrs reperfusion), the gauze pads were removed and their hemoglobin content quantified. These data showed the expected result, in that the least amount of surgical bleeding was detected in the nonanticoagulated animals [FIG. 18A], whereas there was a progressive increase in the amount of surgical blood loss with increasing doses of heparin. Although at the highest dose of Factor IXai tested (600 μg/kg), there was also an increase in surgical bleeding, the two lower doses (including the 300 μg/kg dose which was functionally beneficial) did not result in an increase in surgical blood loss. These data are graphically illustrated by the appearance of representative blood-soaked gauze pads from the surgical wound [FIG. 18B].

Another potential reason for the lack of beneficial effect of heparin or high dose Factor IXai is that intraparenchymal hemorrhage may occur in the postischemic lungs due to friable microvessels. A spectrophotometric assay for hemoglobin, which we have recently validated as a means to quantify intracerebral hemorrhage in a model of stroke, was used to detect residual hemoglobin after flushing the lungs with saline prior to harvest. Although in general, most experimental conditions revealed similar levels of residual hemoglobin content, mice pretreated with 600 U/kg of heparin demonstrated a significant increase in intraparenchymal hemorrhage compared with the other groups [FIG. 19]. This, in addition to the excessive blood loss in the surgical wound itself, may have detracted from what may have otherwise been protective effects due to its antithrombotic actions. Note that there was no increase in intraparenchymal pulmonary hemorrhage at any of the tested doses of Factor IXai.

Although the measurement of bleeding time does not necessarily accurately predict surgical bleeding (18), tail vein bleeding times were nevertheless performed in these experiments because some investigators believe this data is useful. These data show that the therapeutically effective dose of Factor IXai (300 μg/kg) does not increase the tail vein bleeding time, although heparin at an intermediate dose does increase the tail vein bleeding time [FIG. 20).

As oxygen deprivation is a known potent inducer of the production of several important cytokines, many models of tissue ischemia have examined the production of cytokines as surrogate markers of tissue injury. Although the production of pro- or anti-inflammatory cytokines may be incidental to tissue injury or may contribute to it, at the conclusion of these experiments, blood was obtained to determine whether preischemic anticoagulant therapy modulated circulating levels of cytokines. Compared with levels obtained in control animals, ischemia/reperfusion increased the levels of TNF, IL-6, and IL-10 (and there was a tendency for IL-1 b to be increased as well). Although anticoagulant treatment with heparin or Factor IXai did not significantly effect levels of these cytokines compared with ischemia/reperfusion protocol alone, levels of IL-1 were less in the Factor IXai-treated gropu than in the heparin-treated animals.

Discussion

The postischemic vascular milieu is characterized by increases in production of reactive oxygen intermediates, vasoreactivity, leukocyte adhesion, permeability, and thrombosis. In the lungs, these alterations are particularly pronounced, presumably due to the rich vascularity of the lungs and the relatively large surface area over which blood-borne components can interact with endothelium. Although clear roles for leukocyte adhesion receptors have been defined in the setting of frank pulmonary ischemia (19-21), the pathophysiological role for localized thrombosis has been ascribed only by inference. Several clinical scenarios, including global pulmonary ischemia and reperfusion which comes about as a result of lung transplantation, have been characterized by elevations in pulmonary vascular resistance and the occasional echocardiographic observation of large pulmonary venous thrombi (22-24). The studies described here are the first to examine a direct role for localized thrombosis in the pathphysiology of ischemic-reperfused lung injury. To fulfill Koch's postulates, to demonstrate that pulmonary thrombosis is a pathophysiological mediator of ischemic lung injury, these studies demonstrated that (1) thrombosis occurs in the setting of lung ischemia and reperfusion; (2) poor lung function/recipient demise is associated with increased pulmonary thrombosis; and (3) inhibiting pulmonary thrombosis with an agent which does not increase intraparenchymal or surgical bleeding is associated with reduced thrombosis and improved pulmonary function.

The approach taken here to prevent intravascular thrombosis yet leave protective hemostatic mechanisms relatively unimpaired is one in which Factor IXa, inactivated by chemical modification of a critical histidine 221 and serine 376 residue at the active site (25), competes with native Factor IXa for assembly in into the factor IXa-VIIIa-X complex. This approach has the theoretical advantage that it leaves the Tissue Factor-VIIa-mediated activation of Factor X unimpaired. As surgical incisions and subendothelial basement membranes are rife with tissue factor, it is likely that friable postischemic pulmonary vessels and vessels transected during the surgical procedure are still be able to form effective hemostatic plugs. Inhibition of Factor IXa activity is also likely to confer relative anticoagulant selectivity to the intact vessel wall, as stimulated endothelial cells express receptors for Factors IX and X, from which factor IX/IXa would be strategically positioned to participate in the regulation of coagulation within the vessel lumen (26). Furthermore, as formation of the Xase complex is greatly accelerated by the presence of a platelets and their phospholipid surface (27), and platelets are predominantly intravascular in location, this provides another compelling reason why a stratgegy of Factor IXa blockade may be useful in a surgical model. In an electrocautery injury model of canine coronary artery thrombosis (28), this strategy did prevent arterial clot formation. Furthermore, a sufficient degree of anticoagulation was provided by Factor IXa blockade to permit proper function of the cardiopulmonary bypass apparatus without thrombotic obstruction (29). Although others have used active site-blocked factor Xa as an inhibitor of factor Xa assembly into the prothrombinase complex, inhibition of thrombosis was associated with an increase in extravascular bleeding (30).

A strategy of factor IXa blockade, is fundamentally different, however, from one in which heparin is employed as the anticoagulant. Heparin prevents the formation of fibrin from fibrinogen by combining with and activating antithrombin III (AT-III); the heparin/AT-III complex inhibits activated serine proteases at multiple points in the coagulation cascade, including at the level of thrombin (factor IIa) and factor Xa, both of which are critical components of the final common pathway of coagulation. The reason for choosing unfractionated heparin as the comparison compound for these studies was that it is the agent which is most commonly used to treat acute thrombotic disorders in clinical practice, especially in the setting of surgical procedures. Although other anticoagulants are used in discrete settings, none of these agents inhibits coagulation proximal to the level of Factor X (the final common pathway). Low molecular weight heparin (LMWH), for instance, predominately inhibits activated factor Xa] with lesser inhibitory effects on IIa compared with unfractionated heparin (31,32) [, and, unlike heparin is not inhibited by platelet factor IV released from activated platelets (33). Hirudin, a direct thrombin inhibitor, also acts directly on a critical component of the final common pathway (IIa), and therefore would be expected to interfere with surgical hemostasis (34,35). Antiplatelet strategies, such as those involving inhibition of the glycoprotein IIb/IIIa receptor, would similarly be expected to inhibit a common component of the accruing thrombus, fibrinogen-mediated platelet-platelet bridging, which would theoretically have little discriminatory value for intravascular thrombus versus that formed at the cut edge of a wound or at a broken blood vessel.

In summary, administration of a competitive inhibitor of Factor IXa, active-site blocked Factor IXa, is effective to reduce microvascular thrombosis and improve lung function in the setting of lung ischemia/reperfusion injury. The benefits of this particular form of anticoagulant therapy may be conferred because there is a relatively large therapeutic index between doses required for anticoagulant efficacy and those which promote surgical bleeding. It is likely that a therapeutic strategies targetted at formation of the Factor IXa-VIIIa-X activation complex will prove therapeutically useful in a number of thrombotic disorders in which there is a need to limit both in situ thrombosis and hemorrhage at the same time.

### REFERENCES FOR EXAMPLE 8

1. Choudhri, T. F., B. L. Hoh, H.-G. Zerwes, C. J. Prestigiacomo, S. C. Kim, E. S. Jr. Connolly, G. Kottirsch, and D. J. Pinsky. 1998. Reduced microvascular thrombosis and improved outcome in acute murine stroke by inhibiting GPIIb/IIIa receptor-mediated platelet aggregation. J Clin Invest. 102:1301-1310.
2. Sarsam, M. A., N. A. Yonan, D. Beton, D. McMaster, and A. K. Deiraniya. 1993. Early pulmonary vein thrombosis after single lung transplantation. J Heart Lung Transplant 12:17-19.
3. Stang, M. R., A. L. Hinderliter, K. K. Gott, L. J. Paradowski, and R. M. Aris. 1996. Atrial anastomotic thrombus causes neurologic deficits in a lung transplant recipient. Transplant 62 (5):693-695.
4. Shishido, T., S. Beppu, H. Matsuda, and K. Miyatake. 1993. Progression of intramural hemorrhage in the reperfused area: an experimental study assessed by myocardial contrast echocardiography. Circulation 88 (4) Part 2:I-402.
5. Asanuma, T., K. Tanabe, K. Ochiai, H. Yoshitomi, K. Nakamura, Y. Murakami, K. Sano, T. Shimada, R. Murakami, S. Morioka, and S. Beppu. 1997. Relationship between progressive microvascular damage and intramyocardial hemorrhage in patients with reperfused anterior myocardial infarction: myocardial contrast echocardiographic study. Circulation 96 (2):448-453.
6. Nemerson, Y. 1988. Tissue factor and hemostasis. Blood 71(1):1-8.
7. Choudhri, T. F., B. L. Hoh, R. A. Solomon, E. S. Connolly, and D. J. Pinsky. 1997. Use of a spectrophotometric hemoglobin assay to objectively quantify intracerebral hemorrhage in mice. Stroke 28:2296-2302.
8. Van Kampen, E. J. and W. G. Zijlstra. 1961. Standardization of hemoglobinometry II. the hemiglobincyanide method. Clin Chim Acta 6:538-544.
9. Standefer, J. C. and D. Vanderjagt. 1977. Use of tetramethylbenzidine in plasma hemoglobin assay. Clin Chem 23(4):749-751.
10. Bowie, E. J. W. and C. A. Jr. Owen. 1974. The bleeding time. In progress in hemostasis and thrombosis. Grune and Stratton, New York. 249 pp.
11. Francis, C. W., V. J. Marder, and S. E. Martin. 1980. Plasmic degradation of crosslinked fibrin. I. Structural analysis of the particulate clot and identification of new macromolecular-soluble complexes. Blood 56(3):456-464.
12. Lawson, C. A., S-D. Yan, S—F. Yan, H. Liao, G. Chen, J. Sobel, W. Kisiel, D. M. Stern, and D. J. Pinsky. 1997. Monocytes and tissue factor promote thrombosis in a murine model of oxygen deprivation. J Clin Invest 99:1729-1738.
13. Bradford, M. M. 1976. A rapid and sensitive method for the quantification of microgram quantities of protein utilizing the principle of protein-dye binding. Analytical Biochemistry 72:248-254.
14. Hui, K. Y., E. Haber, and G. R. Matsueda. 1983. Monoclonal antibodies to a synthetic fibrin-like peptide bind to human fibrin but not fibrinogen. Science 222 (4628):1129-1132.
15. Naka, Y., N. C. Chowdhury, H. Liao, D. K. Roy, M. C. Oz, R. E. Michler, and D. J. Pinsky. 1995. Enhanced preservation of orthotopically transplanted rat lungs by nitroglycerin but not hydralazine. Requirement for graft vascular homeostasis beyond harvest vasodilation. Circ Res 76:900-906.
16. Pinsky, D. J., H. Liao, C. A. Lawson, S.-F. Yan, J. Chen, P. Carmeliet, D. J. Loskutoff, and D. M. Stern. 1998. Coordinated induction of plasminogen activator inhibitor-1 (PAI-1) and inhibition of plasminogen activator gene expression by hypoxia promotes pulmonary vascular fibrin deposition. J Clin Invest 102:919-928.
17. Yan, S-F., Y. S. Zou, Y. Gao, C. Zhai, N. Mackman, S. L. Lee, J. Milbrandt, D. J. Pinsky, and D. M. Stern. 1998. Tissue factor transcription driven by egr-1 is a critical mechanism of murine pulmonary fibrin deposition in hypoxia. Proc Natl Acad Sci (USA) 95:8298-8303.
18. Lind, S. E. 1991. The bleeding time does not predict surgical bleeding. Blood 77(12):2547-2552.
19. Naka, Y., K. Toda, K. Kayano, M. C. Oz, and D. J. Pinsky. 1997. Failure to express the P-selectin gene or P-selectin blockade confer early pulmonary protection after lung ischemia or transplantation. Proc Natl Acad Sci (USA) 94:757-761.
20. Kapelanski, D. P., A. Iguchi, S. D. Niles, and H-Z. Mao. 1993. Lung reperfusion injury is reduced by inhibiting a CD18-dependent mechanism. J Heart Lung Transplant 12:294-307.
21. Horgan, M. J., S. D. Wright, and A. B. Malik. 1990. Antibody against leukocyte integrin (CD18) prevents reperfusion-induced lung vascular injury. Am J Physiol 259:L315-L319.
22. Adatia, I., C. Lillehei, J. H. Arnold, J. E. Thompson, R. Palazzo, J. C. Fackler, and D. L. Wessel. 1994. Inhaled nitric oxide in the treatment of postoperative graft dysfunction after lung transplantation. Ann Thorac Surg 57:1311-1318.
23. Date, H., A. N. Triantafillou, E. P. Trulock, M. S. Pohl, J. D. Cooper, and G. A. Patterson. 1996. Inhaled nitric oxide reduces human lung allograft dysfunction. J Thorac Cardiovasc Surg 111(5):913-919.
24. Shah, A. S., R. E. Michler, R. J. Downey, D. W. Leibowitz, S. Homma, and C. R. Smith. 1995. Management strategies for pulmonary vein thrombosis following single lung transplantation. J Card Surg 10 (2):169-178.
25. Lollar, P. and D. N. Fass. 1984. Inhibition of activated porcine factor Ix by dansyl-glutamyl-glycyl-arginyl-chloromethylketone. Arch Biochem Biophys 233(2):438-446.
26. Stern, D. M., G. Knitter, W. Kisiel, and P. P. Nawroth. 1987. In vivo evidence of intravascular binding sites for coagulation factor IX. Br J Haematol 66(2):227-232.
27. Sumner, W. T., D. M. Monroe, and M. Hoffman. 1996. Variability in platelet procoagulant activity in healthy volunteers. Thromb Res 81(5):533-543.
28. Benedict, C. R., J. Ryan, B. Wolitzky, R. Ramos, M. Gerlach, P. Tijburg, and D. Stern. 1991. Active site-blocked factor IXa prevents intravascular thrombus formation in the coronary vasculature without inhibiting extravascular coagulation in a canine thrombosis model. J Clin Invest 88(5):1760-1765.
29. Spanier, T. B., M. C. Oz, O. P. Minanov, R. Simantov, W. Kisiel, D. M. Stern, E. A. Rose, and A. M. Schmidt. 1998. Heparinless cardiopulmonary bypass with active-site blocked factor IXa: a preliminary study on the dog. J Thorac Cardiovasc Surg 115(5):1179-1188.
30. Benedict, C. R., J. Ryan, J. Todd, K. Kuwabara, P. Tijburg, J. Jr. Cartwright, and D. Stern. 1993. Active site-blocked factor Xa prevents thrombus formation in the coronary vasculature in parallel with inhibition of extravascular coagulation in a canine thrombosis model. Blood 81 (8):2059-2066.
31. Weitz, J. 1994. New anticoagulant strategies. Current status and future potential. Drugs 48(4):485-497.
32. Messmore, H. L. and W. H. Wehrmacher. 1993. Therapeutic use of low molecular weight heparins. Semin Thromb Hemost 19 Suppl 1:97-100.
33. Lane, D. A., J. Denton, A. M. Flynn, L. Thunberg, and U. Lindahl. 1984. Anticoagulant activities of heparin oligosaccharides and their neutralization by platelet factor 4. Biochem J 218(3):725-732.
34. Rydel, T. J., K. G. Ravichandran, A. Tulinsky, W. Bode, R. Huber, C. Roitsch, and J. W. 2d. Fenton. 1990. The structure of a complex of recombinant hirudin and human alpha-thrombin. Science 249(4966):277-280.
35. Vu, T. K., V. I. Wheaton, D. T. Hung, I. Charo, and S. R. Coughlin. 1991. Domains specifying thrombin-receptor interaction. Nature 353(6345):674-677.

### EXAMPLE 9

### Targeted Inhibition of Intrinsic Coagulation Limits Cerebral Injury in Stroke without Increasing Intracerebral Hemorrhage

Abbreviations: CBF, cerebral blood flow; Factor IXai, active-site blocked factor IXa; ICAM-1, intercellular adhesion molecule-1; ICH, intracerebral hemorrhage; tPA, tissue plasminogen activator; TTC, triphenyl tetrazolium chloride.

Introduction

Agents which restore vascular patency in stroke also increase the risk of intracerebral hemorrhage (ICH). As Factor IXa is a key intermediary in the intrinsic pathway of coagulation, targeted inhibition of Factor IXa-dependent coagulation might inhibit microvascular thrombosis in stroke without impairing extrinsic hemostatic mechanisms which limit ICH. A competitive inhibitor of native Factor IXa for assembly into the intrinsic Factor X activation complex, Factor IXai, was prepared by covalent modification of the Factor IXa active site. In a modified cephalin clotting time assay, in vivo administration of Factor IXai caused a dose-dependent increase in time to clot formation (3.6-fold increase at the 300 μg/kg dose compared with vehicle-treated controls animals, P<0.05). Mice given Factor IXai and subjected to middle cerebral artery occlusion and reperfusion demonstrated reduced microvascular fibrin accumulation by immunoblotting and immunostaining, reduced ¹¹¹In-labelled platelet deposition (42% decrease, P<0.05), increased cerebral perfusion (2.6-fold increase in ipsilateral blood flow by laser doppler, P<0.05), and smaller cerebral infarcts than vehicle-treated controls (70%-fold reduction, P<0.05) based on triphenyltetrazolium chloride staining of serial cerebral sections. At therapeutically effective doses, Factor IXai was not associated with increased ICH, as opposed to tPA or heparin, both of which significantly increased ICH. Factor IXai was cerebroprotective even when given after the onset of stroke, indicating that microvascular thrombosis continues to evolve (and may be inhibited) even after primary occlusion of a major cerebrovascular tributary.

Timely reestablishment of blood flow to ischemic brain using thrombolytic agents represents the current treatment paradigm for acute stroke (1-3). This approach is limited, however, by an increased risk of intracerebral hemorrhage (ICH) and an increase in early mortality (1-5). Furthermore, even if the best available agent, recombinant tissue plasminogen activator (tPA¹) is given promptly, within 3 hours of symptom onset, there is no improvement in overall mortality (1-3,5). Even though there is an improvement in the composite endpoint of death and morbidity with tPA treatment, it is likely that efficacy could be substantially improved if the risk of ICH were lowered. In addition, recent reports indicate that tPA may have a direct role in neuronal injury in the setting of stroke (6-11). Limited anticoagulant trials of heparin in stroke showed heparin to be either ineffective and/or associated with an unacceptably high incidence of hemorrhagic conversion (12-17). In a murine model of stroke, a platelet glycoprotein IIb/IIIa receptor antagonist, although effective at reducing cerebral infarct volumes, caused a dose-dependent increase in ICH (18). Consequently, there remains a clear need to identify new agents which can promote reperfusion without increasing the risk of ICH.

Following an ischemic event, the vascular wall is modified from its quiescent, anti-adhesive, antithrombotic state, to one which promotes leukocyte adhesion and thrombosis. In acute stroke, active recruitment of leukocytes by adhesion receptors expressed in the ipsilateral microvasculature, such as intercellular adhesion molecule-1 (ICA-1) (19) and P-selectin (20), potentiates postischemic hypoperfusion. However, experiments with mice deletionally mutant for each of these genes demonstrate that even in their absence, postischemic cerebral blood flow (CBF) returns only partially to baseline after removal of an intraluminal middle cerebral artery occluding suture. These observations imply the existence of additional mechanisms responsible for postischemic cerebrovascular no-reflow, especially the possibility that local thrombosis occurs at the level of the microvasculature (distal to the site of primary occlusion) in stroke. Furthermore, if the ischemic insult is particularly severe, reflow continues to worsen over the time subsequent to withdrawal of the occluding suture, suggesting ongoing vascular obstructive processes (such as de novo thrombosis) in the distal microvasculature. Recent data in a murine model of stroke implicates GP IIb/IIIa receptor-dependent platelet recruitment as a mechanism which amplifies thrombosis in the postischemic microvasculature (18).

These observations provide the rationale for identifying new strategies to selectively limit thrombosis in stroke without increasing ICH. We hypothesized that anticoagulant strategies which do not impair tissue factor-mediated hemostatic events might reduce thrombosis in the microvascular lumen yet not impair the ability of friable postischemic cerebral microvessels to form effective hemostatic plugs to limit ICH. Heparin or hirudin, which interfere with the final common pathway of coagulation, or thrombolytic agents such as tPA, which lyse fibrin, do not offer the theoretical advantage offered by targeting earlier points in the coagulation cascade. The current experiments test whether selective blockade of IXa/VIIIa/X activation complex assembly using a competitive inhibitor of Factor IXa (active-site blocked IXa, Factor IXai) can limit intravascular thrombosis while preserving mechanisms of extravascular hemostasis in stroke.

Methods

Murine stroke model. Transient focal cerebral ischemia was induced in mice by intraluminal occlusion of the middle cerebral artery (45 minutes) and reperfusion (24 hrs) as previously reported (21). Serial measurements of relative cerebral blood flow (CBF) were recorded via laser doppler flowmetry at previously defined neuroanatomic landmarks (21), and infarct volumes (% ipsilateral hemisphere) determined by planimetric/volumetric analysis of triphenyl tetrazolium chloride (TTC)-stained serial cerebral sections (21).

¹¹¹Indium-platelet studies. Platelet accumulation was determined using ¹¹¹Indium labeled platelets, collected and prepared as previously described (22). Immediately prior to surgery, mice were given 5×10⁶ ¹¹¹In-labeled-platelets intravenously; deposition was quantified after 24 hours by as ipsilateral cpm/contralateral cpm.

Fibrin immunoblotting/immunostaining. The accumulation of fibrin was measured following sacrifice (of fully heparinized animals) using immunoblotting/immunostaining procedures which have been recently described and validated (21). Because fibrin is extremely insoluble, hemispheric brain tissue extracts were prepared by plasmin digestion, then applied to a standard SDS-polyacrylamide gel for electrophoresis, followed by immunoblotting using a polyclonal rabbit anti-human antibody prepared to gamma-gamma chain dimers present in cross-linked fibrin which can detect murine fibrin, with relatively little cross-reactivity with fibrinogen (22). In additional experiments to localize sites of fibrin accumulation, brains were embedded in paraffin, sectioned, and immunostained using the same anti-fibrin antibody.

Spectrophotometric hemoglobin assay and visual ICH score. ICH was quantified by a spectrophotometric-based assay which we have developed and validated (23). In brief, mouse brains were homogenized, sonicated, centrifuged, and methemoglobin in the supernatants converted (using Drabkin's reagent) to cyanomethemoglobin, the concentration of which was assessed by measuring O.D. at 550 nm. For each experiment, the optical density relative to that obtained from a group of control brains is reported.

Preparation and purification of human IXa/IXai. Factor IXai was prepared by applying Proplex (a mixture of human Vitamin-K dependent coagulation factors [Factors II, VII, IX, and X] generously supplied by Dr. Roger Lundblad, Baxter, Duarte Calif.), reconstituted in Tris-buffered saline (TBS) containing CaCl₂ to a column of calcium-dependent anti-human Factor IX monoclonal antibody (CaFIX-1) coupled to Affi-Gel 10 (BioRad, Hercules Calif.) equilibrated at 4° C. with TBS containing CaCl₂ (0.01 M). Following sample application, the column was washed extensively with TBS containing CaCl₂ (0.01 M) and NaCl (0.5 M) and Factor IX was subsequently eluted in Tris-HCl (0.1M; pH 8.0) containing EDTA (0.03 M). Minimal residual contaminants were then removed using Q-Sepharosc Fast Flow chromatography. Factor IX thus purified migrated as a single band on SDS-PAGE in the absence and presence of mercaptoethanol (10%) with an apparent Mr of ≈68 kDa. Factor IX was then activated at 37° C. by incubation with purified human factor XIa (Haematologic Technologies, Inc.) at 1:1000 enzyme:substrate ratio in Tris-HCl (0.05 M; pH 7.5) containing NaCl (0.1 M) and CaCl₂ (0.005 M) for one hour. Purified Factor IXa migrated as a single band in nonreduced SDS-PAGE gels (Mr=45 kDa), and as two bands, corresponding to the heavy and light chains of Factor IXaβ on reduced gels. The latter material was reacted with an 100-fold molar excess of dansyl-glu-gly-arg chloromethlyketone (Calbiochem) for 3 hrs at 37° C., and the mixture dialyzed overnight at 4° C. versus 20,000 volumes of phosphate-buffered saline. The final product, Factor IXai, was devoid of procoagulant activity, migrated identically to untreated Factor IXa on SDS-PAGE, and had no effect on the clotting time of plasma initiated by Factor Xa or thrombin. Factor IXai was used for experiments following filtration (0.2 μm) and chromatography on DeToxi-gel columns (Pierce, Rockford, Ill.). These preparations had no detectable lipopolysaccharide at a protein concentration of 1-2 mg/mL, using the limulus amebocyte assay (Sigma, St. Louis, Mo.). For experiments in which Factor IXai was used, it was given as a single intravenous bolus at the indicated times and at the indicated doses.

Modified Cephalin Clotting Time. Equal volumes of factor IX-deficient plasma (American Diagnostica Inc.) and 0.144 g/100 mL celite in 0.05 M barbital buffer (Sigma) were combined in silicone-coated glass tubes (Sigma) for 2 minutes at 37° C. To this mixture, an equal volume of 1:16 (v/v) cephalin (10 mg/mL, Sigma) in 0.05 M barbital buffer was added, followed by a one-half volume of sample plasma. After the addition of calcium chloride to a final concentration of 0.001 M, the time required for clot formation was determined.

Results

Using a murine model of middle cerebral artery occlusion with an intraluminal vascular suture, which is removed after 45 minutes to initiate reperfusion, the occurrence of microvascular thrombosis distal to the site of primary occlusion was examined. Platelet-rich thrombotic foci occur within the ischemic cerebral hemisphere, as shown by experiments in which ¹¹¹In-labeled platelets were administered to mice immediately prior to ischemia and their accumulation in the ipsilateral hemisphere measured at 24 hours. In animals not subjected to the surgical procedure to create stroke, the presence of platelets was approximately equal between the right and left hemispheres, as would be expected [FIG. IIa, left bar]. However, when animals were subjected to stroke (and received only saline vehicle for control), radiolabeled platelets preferentially accumulated in the ischemic (ipsilateral) hemisphere, compared with significantly less deposition in the contralateral (nonischemic) hemisphere [FIG. 11a, middle bar]. These data support the occurrence of platelet-rich thrombi in the ischemic territory. Another line of evidence also supports the occurrence of microvascular thrombosis in stroke. This data comes from the immunodetection of fibrin, using an antibody directed against a neoepitope on the gamma-gamma chain dimer of cross-linked fibrin. Immunoblots demonstrate a band of increased intensity in the ipsilateral (right) hemisphere of vehicle-treated animals subjected to focal cerebral ischemia and reperfusion [FIG. 11b, “Vehicle”]. To demonstrate that fibrin accumulation was due to the deposition of intravascular fibrin (rather than due to nonspecific permeability changes and exposure to subendothelial matrix), fibrin immunostaining clearly localized the increased fibrin to the lumina of ipsilateral intracerebral microvessels [FIG. 11c, upper two panels]. As an in vivo physiological correlate of microvascular thrombosis, relative cerebral blood flow was measured by laser doppler during the occlusive period as well as after stroke. These data [FIG. 11d, bars labelled “Vehicle”] show that the intraluminal suture technique significantly reduces ipsilateral cerebral blood flow during the occlusive period [FIG. 11d, middle panel]. Blood flow remains depressed even 24 hours after removing the intraluminal occluding suture [FIG. 11d, right panel], corresponding to the platelet, fibrin immunoblot, and fibrin immunostaining data indicating the presence of postischemic microvascular thrombosis.

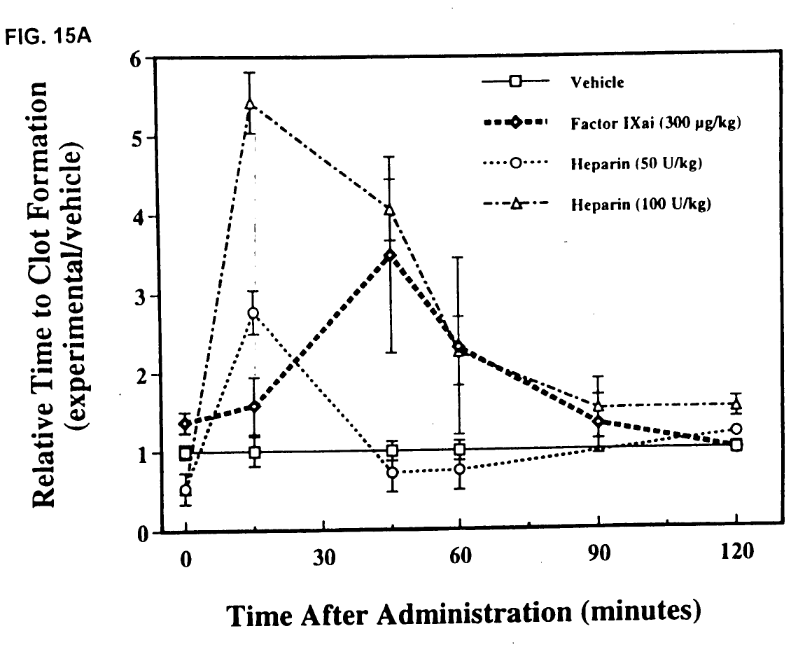

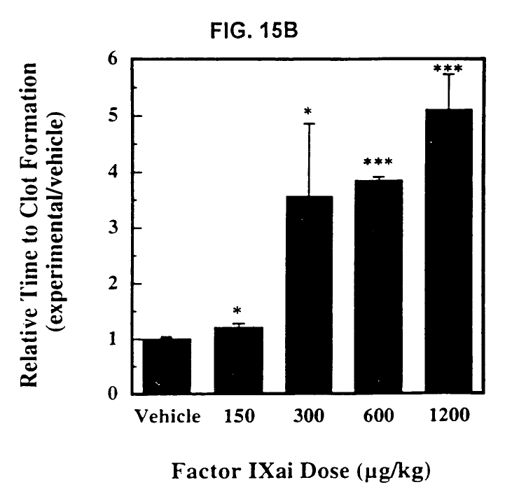

To help establish a functionally deleterious role of microvascular thrombosis in stroke, experiments were performed to test the effect of inhibiting assembly of the Factor IXa/VIIa/X activation complex in vivo. This particular strategy was selected based upon the hypothesis that inhibition of Factor IXa participation in coagulation might inhibit intravascular thrombosis yet not impair tissue factor-VIIa-Xa-mediated extravascular hemostasis (and hence, may not increase intracerebral hemorrhage at clinically effective doses). An estimate of the antithrombotic potency of Factor IXai was obtained by testing mouse plasma in a modified cephalin clotting time assay (MCCT, in which the activity of Factor IXa is a rate-limiting step in thrombus formation) at timed intervals after bolus administration of Factor IXai or control agents. Because of the limited quantity of murine plasma obtained from each sacrificial bleed, plasma was obtained from individual control mice each day this assay was performed (rather than using pooled plasma). Although MCCT control values in mice varied slightly from day to day, the approximate mean control MCCT (for the 15 minute post administration time point) was 150±6 sec (range 108-200 sec). Following administration, Factor IXai demonstrated antithrombotic potency similar to heparin, both of which prolonged the time to clot formation in this assay when compared to control animals that had received a normal saline bolus [FIG. 15a]. The effect of Factor IXai to prolong clotting time in this assay was dose-dependent [FIG. 15b]. To test the in vivo efficacy of Factor IXai in the setting of stroke, Factor IXai was administered to mice immediately prior to stroke, and effects on cerebral microvascular thrombosis, infarct volume, and intracerebral hemorrhage were examined. When Factor IXai (300 μg/kg) is administered to animals prior to introduction of the intraluminal occluding suture, there is a significant reduction in the accumulation of radiolabelled platelets in the ipsilateral hemisphere [FIG. 11a, rightmost bar], no apparent increase in the ipsilateral accumulation of fibrin [FIG. 11b, “Factor IXai”], as well as decreased evidence of intravascular fibrin by immunostaining [FIG. 11c]. In addition, there is a significant increase in postischemic blood flow by this treatment, albeit not completely to preischemic levels [FIG. 11d].

The clinical relevance of these observations is underscored by the striking ability of Factor IXai to reduce cerebral infarct volumes (3.3-fold reduction in infarct volumes at the 300 μg/kg dose, p<0.05) [FIG. 13a]. To test whether this infarct size-reducing property of Factor IXai was unique to this compound, or whether a nonspecific anticoagulant would also demonstrate efficacy in this regard, intravenous heparin was also examined at two doses. Only at the highest dose tested (100 U/kg) did heparin reduce cerebral infarct volumes, however, this was at the cost of a significant increase in intracerebral hemorrhage, measured with a recently validated spectrophotometric assay (23) [FIG. 13b]. In sharp contrast, Factor IXai caused an increase in ICH only at the highest dose tested, but did not do so at doses which demonstrated striking efficacy to reduce cerebral infarct volumes [FIG. 13b]. Because a desirable therapeutic agent in stroke will not only reduce cerebral infarction volumes, but will also minimize ICH, the data shown in FIGS. 3a and b are displayed with infarct volumes plotted along the ordinate and intracerebral hemorrhage plotted along the abscissa [FIG. 13c]. As can be seen in the figure, Factor IXai is able to minimize both infarction volumes and ICH (lower left hand corner of plot), while only the high dose heparin is able to reduce infarct volumes, but at the cost of increasing ICH.

To compare these results with a current therapy for clinical stroke in humans, tPA, experiments were performed in which tPA was administered to mice subjected to stroke and reperfusion. Intravenous tPA at doses of 0.5, 1.0, or 2.0 mg/kg (n=6, 11, and 4, respectively) or vehicle (n=16) were administered to mice in the post-occlusion period immediately after withdrawal of the occluding suture. Data was not collected for animals treated with tPA prior to occlusion because of excessive bleeding associated with the operative procedure mandated by the stroke model. At the three doses examined, tPA demonstrated only trends towards reductions in infarct size compared to vehicle-treated control animals (1.9-fold, 1.6-fold, and 1.3-fold reductions for the 0.5, 1.0, and 2.0 mg/kg doses, respectively), however, none of these reductions was statistically significant. On the other hand, administration of tPA at all doses caused statistically significant increases in ICH (1.7, 1.4, and 2.4-fold increase respectively for the three doses, p=0.01, 0.03, and 0.002, respectively). These data therefore showed no significant reductions in cerebral infarction volumes (although there were trends in this direction) and increased ICH with tPA. These data are in concordance with the recent report that tPA given to tPA-deficient (−/−) or wild-type mice does not improve and may exacerbate cerebral injury in stroke (24).

Because therapies directed at improving outcome from acute stroke must be given after clinical presentation, and because fibrin continues to form following the initial ischemic event in stroke, we tested whether Factor IXai might be effective when given following initiation of cerebral ischemia. Factor IXai given after middle cerebral artery occlusion (following removal of the occluding suture) provided significant cerebral protection judged by its ability to significantly reduce cerebral infarction volumes compared with vehicle-treated controls [FIG. 14].

Discussion

The data in these studies demonstrate clear evidence of intravascular thrombus formation (both platelets and fibrin) within the post-ischemic cerebral microvasculature. In fact, the ability of an anticoagulant such as Factor IXai to improve outcome even when given after the onset of the reperfusion phase suggests that the process of microvascular thrombosis is not limited to that which occurs during the major occlusive event. Rather, microvascular thrombosis appears to be a dynamic process which continues to evolve even after recanalization of the major vascular tributary. The pathophysiological relevance of microvascular thrombosis in stroke is underscored by the ability of Factor IXai to reduce microvascular thrombosis (both platelet and fibrin accumulation are reduced, with an attendant increase in postischemic CBF) and to improve stroke outcome. At clinically relevant doses, treatment with Factor IXai does not cause an increase in ICH, in sharp contrast to tPA in this same model of stroke, in which tPA did not significantly reduce infarct volumes and also increased the degree of ICH. These data suggest that selective inhibition of Factor IXa/VIIIa/X activation complex assembly with Factor IXai is a logical target for stroke therapy in humans. In addition, the potent antithrombotic actions of Factor IXai are likely to be clinically significant in the setting of stroke, because Factor IXai reduces infarct volumes even when given after the onset of stroke.

There are a number of reasons why targetted anticoagulant strategies might be superior to the current use of thrombolytic agents in the management of acute stroke, which have had checkered success in clinical trials. Theoretically, an ideal treatment for acute stroke would prevent the formation or induce dissolution of the fibrin-platelet mesh that causes microvascular thrombosis in the ischemic zone without increasing the risk of intracerebral hemorrhage. However, thrombolytic agents which have been studied in clinical trials of acute stroke have consistently increased the risk of intracerebral hemorrhage (1-5). Streptokinase, given in the first several (<6) hours following stroke onset, was associated with an increased rate of hemorrhagic transformation (up to 67%); although there was increased early mortality, surviving patients suffered less residual disability. A recent meta-analysis of evidence on thrombolytic therapy for acute ischemic stroke shows that, when the major tPA trials are considered, there was a 2.99-fold increase in symptomatic ICH, and when all thrombolytics trials were analyzed, there is a 3.62-fold increase in symptomatic ICH (5). In addition to the potential increased hemorrhagic risk with tPA, there is also the risk of therapeutic failure; platelets continue to be activated during administration of tPA, which may account for some of the therapeutic failures observed with tPA administration. In addition, tPA is short-lived, which may limit its usefulness if microvascular thrombus continues to accrue well beyond its therapeutic half-life. Although tPA is the best among available thrombolytic agents in terms of improving morbidity in clinical stroke, there remains the concern that tPA has been shown to directly mediate excitotoxic neuronal cell injury via extracellular tPA-catalyzed proteolysis of nonfibrin substrates (6-11).

Because of the usually precipitous onset of ischemic stroke, therapy has been targeted primarily towards lysing the major fibrinous/atheroembolic debris which occludes a major vascular tributary to the brain. However, the current work reinforces the previous observation (18) that there is an important component of microvascular thrombosis which occurs downstream from the site of original occlusion. This is likely to be of considerable pathophysiological significance for post-ischemic hypoperfusion (no-reflow) and cerebral injury in evolving stroke. These data are in excellent agreement with those which have been previously reported, in which microthrombi have been topographically localized to the ischemic region in fresh brain infarcts (25). The use of an agent which inhibits assembly of the Factor IXa/VIIIa/X activation complex represents a novel approch to limiting thrombosis which occurs within microvascular lumena, without impairing extravascular hemostasis, the maintenance of which may be critical for preventing ICH. In the current studies, treatment with Factor IXai reduces microvascular platelet and fibrin accumulation, improves postischemic cerebral blood flow, and reduces cerebral infarct volumes in the setting of stroke without increasing ICH. These data, along with those in the current manuscript, show a critical role for platelet accumulation at these downstream sites in cerebral microvascular thrombosis. It is not surprising that Factor IXai inhibits platelet accumulation in stroke, because Factor IXa has an integral role in promoting coagulation via the intrinsic pathway; Factor IXai competes with native Factor IXa for assembly into the tenase complex, and therefore causes competitive inhibition of tenase complex formation. Although this mechanism theoretically should not interfere directly with platelet adhesion, in vivo, coagulation reactions, platelet activation, and leukocyte recruitment all occur in close proximity (as well as in proximity to the vessel wall) and are highly interdependent. This is especially likely to be true in cerebral microvessels following ischemia, where blood flow and dissipation of activated products will be sluggish. Therefore, it is likely that local generation of thrombin (by Factor IXa-dependent coagulation) will locally activate and recruit platelets, as thrombin is a potent activator of platelets.

The studies shown here demonstrate that Factor IXa-mediated coagulation does participate in platelet recruitment, because when Factor IXa-dependent coagulation is inhibited, platelet recruitment is reduced by nearly half. This data does not allow us to extrapolate that initial platelet activation is the sole cause of postischemic microvascular thrombosis; rather, it is likely that the phenotype of the endovascular wall changes, perhaps by diminution of NO levels, perhaps by tissue factor expression in recruited mononuclear phagocytes, perhaps by alterations in the fibrinolytic balance, which all lead to a prothrombotic phenotype. Under these circumstances, even inactivated platelets passing by may become activated and deposit locally. Regardless of the relative importance of platelet accumulation versus fibrin formation in the development of microvascular thrombosis in stroke, the data are clear that the use of an agent which inhibits assembly of the Factor IXa/VIIIa/X activation complex represents an effective approach to limiting thrombosis which occurs within microvascular lumena. In the setting of murine stroke, treatment with Factor IXai reduces microvascular platelet and fibrin accumulation, improves postischemic cerebral blood flow, and reduces cerebral infarct volumes. This approach is even more salient in stroke because it is effective without impairing extravascular hemostasis, the maintenance of which may be critical for preventing ICH.

The potency of Factor IXai as an inhibitor of coagulation stems from the integral role of activated Factor IX in the coagulation cascade. Patients with hemophilia B (“Christmas disease”) are deficient in Factor IX and exhibit hemorrhagic tendencies (26). However, inhibition of Factor IXa-mediated coagulation may be therapeutically useful in discrete circumstances. For the studies shown here, active site-blocked Factor IXa was shown to be a competitive inhibitor of Factor IXa-mediated coagulation in vitro using a modified cephalin clotting time (MCCT) assay instead of the standard activated partial thromboplastin time (APTT). The MCCT assay was used because the sensitivity of the APTT is not sufficient to detect the anticoagulant effect of IXai; for example, administration of Factor IXai (300 μg/kg) did not significantly alter the APTT [79.9±8.9 sec vs 70.6±8.9 sec APTT for IXai-treated (n=7) and vehicle-treated (n=4) mice, respectively, P=NS]. In order to increase the sensitivity of the standard activated partial thromboplastin time (APTT), the amount of “phospholipid”(cephalin) in the incubation mixture in the MCCT was decreased; theoretically, this resulted in a limiting amount of phospholipid. Using the MCCT, studies showed that increased levels of IXai prolonged the clotting time in a Factor IXai-dose-dependent manner. The fact that there is a dose-dependent inhibition of Factor IXa-mediated coagulation by Factor IXai is not unexpected, because Factor IXai acts as a competitive inhibitor of assembly of the tenase complex. We would expect that after a point (that at which all Factor IXa activity is inhibited), we would see no further anticoagulant effect; however, the in vivo dose-response curves show that up to a dose of 1200 μg/kg, we are not at that point.

In addition to its clear-cut efficacy in stroke, active site-blocked Factor IXa has also been shown to be useful in several other quite different in vivo models. In cardiopulmonary bypass, administration of Factor IXai alone (without heparin) was sufficient to maintain patency of the circuit (27). Factor IXai also appears to be effective at preventing progressive coronary artery occlusion induced following the initial application of electric current to the left circumflex coronary artery in dogs (28). This is consistent with the high thrombotic potency of Factor IXa in a Wessler stasis model (29). On the other hand, any new therapy for stroke should be greeted with cautious enthusiasm. Although the therapeutic window for Factor IXai is high (doses which increase ICH are substantially higher than those required for therapeutic efficacy), there is a potential for excessive inhibition of Factor IXa to promote ICH. For instance, protease nexin-2/amyloid beta protein precursor is a potent inhibitor of Factor IXa which accumulates extensively in the cerebral blood vessels of patients with amyloidosis Dutch-type with hereditary cerebral hemorrhage and may be a factor in the development of spontaneous ICH in these patients (30).

The data which demonstrate that IXai given after the onset of stroke is effective leads to another interesting hypothesis, that the formation of thrombus represents a dynamic equilibrium between the processes of ongoing thrombosis and ongoing fibrinolysis. Even under normal (nonischemic) settings, this dynamic equilibrium has been shown to occur in man (31). The data in the current studies, which show that Factor IXai is effective even when administered after the onset of stroke, suggests that this strategy restores the dynamic equilibrium, which is shifted after cerebral ischemia to favor thrombosis, back towards a more quiescent (antithrombotic) vascular wall phenotype.

As a final consideration, even if thrombolysis successfully removes the major occluding thrombus, and/or anticoagulant strategies are effective to limit progressive microcirculatory thrombosis, blood flow usually fails to return to pre-ischemic levels. This is exemplified by data in the current study, in which although cerebral blood flow is considerably improved by Factor IXai (which limits fibrin/platelet accumulation), cerebral blood flow still does not return to preischemic levels. These data support the existence of multiple effector mechanisms for postischemic cerebral hypoperfusion, including postischemic neutrophil accumulation and consequent microvascular plugging with enhanced P-selectin and ICAM-1 expression by cerebral microvascular endothelial cells (19,20). Even when these adhesion receptors are absent as is the case in mice deletionally mutant for these receptors, cerebral blood flow levels are improved following stroke compared with controls but do not return to preischemic levels. These data show that both leukocytes and thrombosis play a role in postischemic cerebral no-reflow, although the interactions between leukocyte and platelet recruitment and thrombosis in vivo are likely to be highly complex, with both positive (32-34) and negative (35) interactions.

In summary, administration of a competitive inhibitor of Factor IXa, active-site blocked Factor IXa, represents a novel therapy for the treatment of stroke. This therapy not only reduces microcirculatory thrombosis, improves postischemic cerebral blood flow, and reduces cerebral tissue injury following stroke, but it can do so even if given after the onset of cerebral ischemia and without increasing the risk of ICH. This combination of infarct size reduction and relatively low downside risk of ICH makes this an extremely attractive approach for further testing and potential clinical trials in human stroke.

### REFERENCES FOR EXAMPLE 9

1. The National Institute of Neurological Disorders and Stroke rt-PA Stroke Study Group, 1995. Tissue plasminogen activator for acute ischemic stroke. N. Engl. J. Med. 333:1581.
2. Hacke, W., M. Kaste, C. Fieschi, D. Toni, E. Lesaffre, R. von Kummer, G. Boysen, E. Bluhmki, G. Hoxter, M. H. Mahagne, M. Hennerici, and for the ECASS Study Group. 1995. Intravenous thrombolysis with recombinant tissue plasminogen activator for acute hemispheric stroke. J. A. M. A. 274 (13):1017.
3. del Zoppo, G. J. 1995. Acute stroke—on the threshold of a therapy. N. Engl. J. Med. 333(13):1632.
4. Hommel, M., C. Cornu, F. Boutitie, J. P. Boissel, and The MultiCenter Acute Stroke Trial—Europe Study Group. 1996. Thrombolytic therapy with streptokinase in acute ischemic stroke. N. Engl. J. Med. 335:145.
5. Wardlaw, J. M., C. P. Warlow, and C. Counsell. 1997. Systematic review of evidence on thrombolytic therapy for acute ischaemic stroke [see comments]. Lancet 350:607.
6. Tsirka, S. E., T. H. Bugge, J. L. Degen, and S. Strickland. 1997. Neuronal death in the central nervous system demonstrates a non-fibrin substrate for plasmin. Proceedings of the National Academy of Sciences USA 94:9779.
7. Tsirka, S. E., A. D. Rogove, T. H. Bugge, J. L. Degen, and S. Strickland. 1997. An extracellular proteolytic cascade promotes neuronal degeneration in the mouse hippocampus. The Journal of Neuroscience 17:543.
8. Korninger, C. and D. Collen. 1981. Studies on the specific fibrinolytic effect of human extrinsic (tissue-type) plasminogen activator in human blood and in various animal species in vitro. Thrombosis Haemostasis 46:561.
9. Tsirka, S. E., A. D. Rogove, and S. Strickland. 1996. Neuronal cell death and tPA. Nature 384:123.
10. Sappino, A.-P., R. Madani, J. Huarte, D. Belin, J. Z. Kiss, A. Wohiwend, and J.-D. Vassalli. 1993. Extracellular proteolysis in the adult murine brain. Journal of Clinical Investigation 92:679.
11. Tsirka, S. E., A. Gualandris, D. G. Amaral, and S. Strickland. 1995. Excitotoxin-induced neuronal degeneration and seizure are mediated by tissue plasminogen activator. Nature 377:340.
12. Duke R J, Bloch R F, Turpie A G G, Trebilcock R, and Bayer N. 1996. Intravenous heparin for the prevention of stroke progression in acute partial stable stroke: a randomized controlled trial. Annals of Internal Medicine 105:825.
13. Haley E C Jr., Kassell N F, and Torner J C. 1988. Failure of heparin to prevent progession in progressing ischemic infarction. Stroke 19:10.
14. Slivka A, and Levy D. 1990. Natural history of progressive ischemic stroke in a population treated with heparin. Stroke 21:1657.
15. Ramirez-Lassepas M, Quifiones M R, and Nino H H. 1986. Treatment of acute ischemic stroke: open trial with continuous intravenous heparinization. Arch. Neurol. 43:386.
16. Bogousslavsky J, and Regli F. 1985. Anticoagulant-induced intracerebral bleeding in brain ischemia: evaluation in 200 patients with TIAs, emboli from the heart, and progressing stroke. Acta Neurol Scand 71:464.
17. Cerebral Embolism Study Group, 1984. Immediate anticoagulation of embolic stroke: brain hemorrhage and management options. Stroke 15:779.
18. Choudhri, T. F., B. L. Hoh, H.-G. Zerwes, C. J. Prestigiacomo, S. C. Kim, E. S. Jr. Connolly, G. Kottirsch, and D. J. Pinsky. 1998. Reduced microvascular thrombosis and improved outcome in acute murine stroke by inhibiting GP IIb/IIIa receptor-mediated platelet aggregation. J Clin Invest 102:1301.
19. Connolly, E. S. Jr., C. J. Winfree, T. A. Springer, Y. Naka, H. Liao, S. D. Yan, D. M. Stern, R. A. Solomon, J-C. Gutierrez-Ramos, and D. J. Pinsky. 1996. Cerebral protection in homozygous null ICAM-1 mice after middle cerebral artery occlusion. Role of neutrophil adhesion in the pathogenesis of stroke. J. Clin. Invest. 97:209.
20. Connolly, E. S. Jr., C. J. Winfree, C. J. Prestigiacomo, S. C. Kim, T. F. Choudhri, B. L. Hoh, Y. Naka, R. A. Solomon, and D. J. Pinsky. 1997. Exacerbation of cerebral injury in mice which express the P-selectin gene: identification of P-selectin blockade as a new target for the treatment of stroke. Circ. Res. 81:304.
21. Connolly, E. S. Jr., C. J. Winfree, D. M. Stern, R. A. Solomon, and D. J. Pinsky. 1996. Procedural and strain-related variables significantly affect outcome in a murine model of focal cerebral ischemia. Neurosurgery 38 (3):523.
22. Naka, Y., N. C. Chowdhury, H. Liao, D. K. Roy, M. C. Oz, R. E. Micheler, and D. J. Pinsky. 1995. Enhanced preservation of orthotopically transplanted rat lungs by nitroglycerin but not hydralazine: requirement for graft vascular homeostasis beyond harvest vasodilation. Circ. Res. 76:900.
23. Choudhri, T. F., B. L. Hoh, R. A. Solomon, E. S. Jr. Connolly, and D. J. Pinsky. 1997. Use of a spectrophotometric hemoglobin assay to objectively quantify intracerebral hemorrhage in mice. Stroke 28:2296.
24. Wang, Y. F., S. E. Tsirka, S. Strickland, P. E. Steig, S. G. Soriano, and S. A. Lipton. 1998. Tissue plasminogen activator (tPA) increases neuronal damage after focal cerebral ischemia in wild-type and tPA-deficient mice. Nature Medicine 4:228.
25. Heye, N., C. Paetzold, R. Steinberg, and J. Cervos-Navarro. 1992. The topography of microthrombi in ischemic brain infarct. Acta Neurologica Scandinavica 86:450.
26. Taran, L. D. 1997. Factor IX of the blood coagulation system: a review. Biochemistry 62:685.
27. Spanier, T. B., J. M. Chen, M. C. Oz, N. M. Edwards, W. Kisiel, E. A. Rose, and A. M. Schmidt. 1998. Selective anticoagulation with active site-blocked Factor IXa suggests separate roles for intrinsic and extrinsic coagulation pathways in cardiopulmonary bypass. J. Thorac. Cardiovasc. Surg. 116:860.
28. Benedict, C. R., J. Ryan, B. Wolitzky, R. Ramos, M. Gerlach, P. Tijburg, and D. Stern. 1991. Active site-blocked Factor IXa prevents intravascular thrombus formation in the coronary vasculature without inhibiting extravascular coagulation in a canine thrombosis model. J. Clin. Invest. 88:1760.
29. Gray, E., J. Tubbs, S. Thomas, A. Oates, M. Boisclair, G. Kemball-Cook, and T. W. Barrowcliffe. 1995. Measurement of activated factor IX in factor IX concentrates: correlation with in vivo thrombogenicity. Thromb Haemost 73:675.
30. Schmaier, A. H., L. D. Dahl, A. J. Rozemuller, R. A. Roos, S. L. Wagner, R. Chung, and W. E. Van Nostrand. 1993. Protease nexin-2/amyloid beta protein precursor. A tight-binding inhibitor of coagulation factor IXa. J. Clin. Invest. 92:2540.
31. Nossel, H. L. 1981. Relative proteolysis of the fibrinogen B beta chain by thrombin and plasmin as a determinant of thrombosis. Nature 291:165.
32. Furie, B. and B. C. Furie. 1995. The molecular basis of platelet and endothelial cell interaction with neutrophils and monocytes: role of P-selectin and the P-selectin ligand, PSGL-1. Thrombosis and Haemostasis 74:224.
33. Palabrica, T., R. Lobb, B. C. Furie, M. Aronovitz, C. Benjamin, Y.-M. Hsu, S. A. Sajer, and B. Furie. 1992. Leukocye accumulation promoting fibrin deposition is mediated in vivo by P-selectin. Nature 359:848.
34. Larsen, E., A. Celi, G. Gilbert, B. C. Furie, J. Erban, R. Bonfanti, D. D. Wagner, and B. Furie. 1989. PADGEM protein: a receptor that mediates the interaction of activated platelets with neutrophils and monocytes. Cell 59:305.
35. Valles, J., M. T. Santos, A. J. Marcus, L. B. Safier, M. J. Broekman, N. Islam, H. L. Ullman, and J. Aznar. 1993. Downregulation of human platelet reactivity by neutrophils: participation of lipoxygenase derivatives and adhesive proteins. J. Clin. Invest. 92:1357.

### EXAMPLE 10

### Inhibition of Factor IXa-Dependent Coagulation Improves Efficacy of tPA in Stroke without Increasing Intracerebral Hemorrhage

Treatment of stroke with tissue plasminogen activator (tPA) is limited by intra cerebral hemorrhage (ICH) and reflow failure. We hypothesize that inhibiting intrinsic but not extravascular (tissue-factor/VI Ia-mediated) coagulation might permit dose-reductions of tPA without increasing ICH. Active-site blocked factor IXa (IXai) caused dose-dependent inhibition of coagulation in a modified cephalin clotting time assay (3.5 fold ↑ clotting time at 300 μg/kg, p<0.05 vs. control). As our recent work shows that Ixai improves stroke outcome at doses ≧300 μg/kg, a dose of IXai which does not improve outcome in murine stroke (150 μg/kg) was used in combination with tPA. Mice were given Factor Ixai (150 μg/kg) or vehicle (Veh) just prior to middle cerebral artery occlusion (45 min), and graded doses of tPA were administered at the time of occluding suture withdrawal. Outcomes included; cerebral infarct volume (% ipsilateral hemisphere); cerebral blood flow (CBF, % contralateral laser dopler flow) immediately after treatment (CBF₁) and at 24 hrs (CBF₁); and ICH (ipsilateral/contralateral cyanomethemoglobin content). In contrast to IXai, heparin alone (50-100 U/kg) increased ICH by 1.5-2.5 fold. tPA alone does note improve CBF₁ increases ICH, and does not ↑ infarct volumes. Low dose IXai permits the use of reduced doses of tPA, simultaneously decreasing infarct volumes and improving reperfusion without increasing ICH.

TABLE V

| | | | | | IXai + |
|---|---|---|---|---|---|
| | Veh | tPA, 0.5 | tPA, 1.0 | tPA, 2.0 | tPA, 0.5 |
| n | 31 | 8 | 15 | 7 | 10 |
| CBFᵢ | 43 ± 4 | 43 ± 5 | 55 ± 7 | 55 ± 9 | 70 ± 4* |
| CBF_{f} | 29 ± 2 | 51 ± 6* | 35 ± 4 | 37 ± 7 | 62 ± 5** |
| Infarct | 21 ± 3 | 11 ± 4 | 13 ± 2 | 17 ± 6 | 10 ± 3* |
| Vol | | | | | |
| ICH | 1 ± 0 | 1.7 ± 0.3* | 1.4 ± 0.1* | 2.4 ± 0.5** | 1 ± 0 |

tPA doses mg/kg; IXai 150 mg/kg; Means ± _SEM,
*P < 0.05,
**p < 0.005 vs Veh

1. A method for treating an ischemic disorder in a subject which comprises administering to the subject a pharmaceutically acceptable form of a Factor IXa, compound in a sufficient amount over a sufficient period of time to inhibit coagulation so as to treat the ischemic disorder in the subject.

2. A method for treating an ischemic disorder in a subject which comprises administering to the subject a pharmaceutically acceptable form of a Factor IXa compound and a pharmaceutically acceptable form of an indirect or direct fibrinolytic agent, each in a sufficient amount over a sufficient period of time to inhibit coagulation so as to treat the ischemic disorder in the subject.

3-20. (canceled)

21. A method for identifying a compound that is capable of improving an ischemic disorder in a subject which comprises:
a) administering the compound to an animal, which animal is a stroke animal model;
b) measuring stroke outcome in the animal; and
c) comparing the stroke outcome in step (b) with that of the stroke animal model in the absence of the compound so as to identify a compound capable of improving an ischemic disorder in a subject.

22-24. (canceled)

25. A method for treating a reperfusion injury in a subject which comprises administering to the subject a Factor IXa compound in a sufficient amount over a sufficient period of time to inhibit coagulation so as to treat the reperfusion injury in the subject.

26-28. (canceled)

29. A method of inhibiting clot formation in a subject which comprises adding to blood an amount of an inactive recombinant mutein in an amount effective to inhibit clot formation in the subject but which does not significantly interfere with hemostasis when the blood is administered to a patient.

30. (canceled)

31. An assay to monitor the effect of a Factor IXa compound administered to a subject to treat an ischemic disorder in the subject which comprises:
a) measuring the ischemic disorder in the subject;
b) administering the factor IXa compound to the subject and measuring the ischemic disorder; and
c) comparing the measurement of the ischemic disorder in step (b) with that measured in step (a) so as to monitor the effect of the Factor IXa compound.

32. (canceled)
